SEQUENCE LISTING (116 sequences)

SEQ ID NO: 1 - protein, 10 aa, Homo sapiens
GVYDGREHTV

SEQ ID NO: 2 - protein, 7 aa, Homo sapiens
TSDQSYG

SEQ ID NO: 3 - protein, 8 aa, Homo sapiens
QGSYDEQN

SEQ ID NO: 4 - protein, 14 aa, Homo sapiens
CAMSGDSAGNMLTF

SEQ ID NO: 5 - protein, 5 aa, Homo sapiens
KGHDR

SEQ ID NO: 6 - protein, 6 aa, Homo sapiens
SFDVKD

SEQ ID NO: 7 - protein, 17 aa, Homo sapiens
CATSDWDRSGDKETQYF

SEQ ID NO: 8 - protein, 136 aa, Homo sapiens
MSLSSLLKVVTASLWLGPGIAQKITQTQPGMFVQEKEAVTLDCTYDTSDQSYGLFWYKQPSSGEMIFLIYQGSYDEQNATEGRYSLNFQKARKSANLVISASQLGDSAMYFCAMSGDSAGNMLTFGGGTRLMVKPH

SEQ ID NO: 9 - protein, 135 aa, Homo sapiens
MASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATSDWDRSGDKETQYFGPGTRLLVL

SEQ ID NO: 10 - protein, 276 aa, synthetic construct
  REGION 1..276: note = with mininmal murinized constant region
MSLSSLLKVVTASLWLGPGIAQKITQTQPGMFVQEKEAVTLDCTYDTSDQSYGLFWYKQPSSGEMIFLIYQGSYDEQNATEGRYSLNFQKARKSANLVISASQLGDSAMYFCAMSGDSAGNMLTFGGGTRLMVKPHIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLSVIGFRILLLKVAGFNLLMTLRLWSS

SEQ ID NO: 11 - protein, 312 aa, synthetic construct
  REGION 1..312: note = with minimal murinized constant region
MASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATSDWDRSGDKETQYFGPGTRLLVLEDLNKVFPPEVAVFEPSKAEIAHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDF

SEQ ID NO: 12 - protein, 7 aa, Homo sapiens
TSDPSYG

SEQ ID NO: 13 - protein, 8 aa, Homo sapiens
QGSYDQQN

SEQ ID NO: 14 - protein, 15 aa, Homo sapiens
CAMSGGYTGGFKTIF

SEQ ID NO: 15 - protein, 5 aa, Homo sapiens
SGDLS

SEQ ID NO: 16 - protein, 6 aa, Homo sapiens
YYNGEE

SEQ ID NO: 17 - protein, 13 aa, Homo sapiens
CASSGGDGDEQFF

SEQ ID NO: 18 - protein, 137 aa, Homo sapiens
MSLSSLLKVVTASLWLGPGIAQKITQTQPGMFVQEKEAVTLDCTYDTSDPSYGLFWYKQPSSGEMIFLIYQGSYDQQNATEGRYSLNFQKARKSANLVISASQLGDSAMYFCAMSGGYTGGFKTIFGAGTRLFVKAN

SEQ ID NO: 19 - protein, 131 aa, Homo sapiens
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSGGDGDEQFFGPGTRLTVL

SEQ ID NO: 20 - protein, 277 aa, synthetic construct
  REGION 1..277: note = with minimal murinized constant region
MSLSSLLKVVTASLWLGPGIAQKITQTQPGMFVQEKEAVTLDCTYDTSDPSYGLFWYKQPSSGEMIFLIYQGSYDQQNATEGRYSLNFQKARKSANLVISASQLGDSAMYFCAMSGGYTGGFKTIFGAGTRLFVKANIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLSVIGFRILLLKVAGFNLLMTLRLWSS

SEQ ID NO: 21 - protein, 310 aa, synthetic construct
  REGION 1..310: note = with minimal murinized constant region
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSGGDGDEQFFGPGTRLTVLEDLKNVFPPEVAVFEPSKAEIAHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSRSYHQGVLSATILYEILLGKATLYAVLVSALVLMAMVKRKDSRG

SEQ ID NO: 22 - protein, 6 aa, Homo sapiens
DSASNY

SEQ ID NO: 23 - protein, 7 aa, Homo sapiens
IRSNVGE

SEQ ID NO: 24 - protein, 14 aa, Homo sapiens
CAASRGTGFQKLVF

SEQ ID NO: 25 - protein, 5 aa, Homo sapiens
LGHDT

SEQ ID NO: 26 - protein, 6 aa, Homo sapiens
YNNKEL

SEQ ID NO: 27 - protein, 16 aa, Homo sapiens
CASSQFWDGAGDEQYF

SEQ ID NO: 28 - protein, 133 aa, Homo sapiens
MTSIRAVFIFLWLQLDLVNGENVEQHPSTLSVQEGDSAVIKCTYSDSASNYFPWYKQELGKRPQLIIDIRSNVGEKKDQRIAVTLNKTAKHFSLHITETQPEDSAVYFCAASRGTGFQKLVFGTGTRLLVSPN

SEQ ID NO: 29 - protein, 134 aa, Homo sapiens
MGCRLLCCVVFCLLQAGPLDTAVSQTPKYLVTQMGNDKSIKCEQNLGHDTMYWYKQDSKKFLKIMFSYNNKELIINETVPNRFSPKSPDKAHLNLHINSLELGDSAVYFCASSQFWDGAGDEQYFGPGTRLTVT

SEQ ID NO: 30 - protein, 273 aa, synthetic construct
  REGION 1..273: note = with minimal murinized constant region
MTSIRAVFIFLWLQLDLVNGENVEQHPSTLSVQEGDSAVIKCTYSDSASNYFPWYKQELGKRPQLIIDIRSNVGEKKDQRIAVTLNKTAKHFSLHITETQPEDSAVYFCAASRGTGFQKLVFGTGTRLLVSPNIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLSVIGFRILLLKVAGFNLLMTLRLWSS

SEQ ID NO: 31 - protein, 311 aa, synthetic construct
  REGION 1..311: note = with minimal murinized constant region
MGCRLLCCVVFCLLQAGPLDTAVSQTPKYLVTQMGNDKSIKCEQNLGHDTMYWYKQDSKKFLKIMFSYNNKELIINETVPNRFSPKSPDKAHLNLHINSLELGDSAVYFCASSQFWDGAGDEQYFGPGTRLTVTEDLNKVFPPEVAVFEPSKAEIAHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDF

SEQ ID NO: 32 - DNA, 21 bp, synthetic construct
  misc_feature 1..21: note = Codon optimized
  source 1..21: mol_type = other DNA
accagcgatcagagctacggc

SEQ ID NO: 33 - DNA, 24 bp, synthetic construct
  misc_feature 1..24: note = codon optimized
  source 1..24: mol_type = other DNA
cagggcagctacgacgagcagaat

SEQ ID NO: 34 - DNA, 42 bp, synthetic construct
  misc_feature 1..42: note = codon optimized
  source 1..42: mol_type = other DNA
tgtgccatgagcggcgatagcgccggcaacatgcttacattt

SEQ ID NO: 35 - DNA, 15 bp, synthetic construct
  misc_feature 1..15: note = codon optimized
  source 1..15: mol_type = other DNA
aagggccacgaccgg

SEQ ID NO: 36 - DNA, 18 bp, synthetic construct
  misc_feature 1..18: note = codon optimized
  source 1..18: mol_type = other DNA
agcttcgacgtgaaggac

SEQ ID NO: 37 - DNA, 51 bp, synthetic construct
  misc_feature 1..51: note = codon optimized
  source 1..51: mol_type = other DNA
tgcgccaccagcgactgggatagaagcggcgacaaagagacacagtacttc

SEQ ID NO: 38 - DNA, 831 bp, synthetic construct
  misc_feature 1..831: note = with minimal murinized constant region; codon optimized
  source 1..831: mol_type = other DNA
atgagcctgagcagcctgctgaaggtcgtgacagcctctctgtggctcggacctggaatcgcccagaagatcacccagacacagcccggcatgttcgtgcaagagaaagaagccgtgacactggactgcacctacgacaccagcgatcagagctacggcctgttctggtacaagcagcctagcagcggcgagatgatcttcctgatctaccagggcagctacgacgagcagaatgccaccgagggcagatacagcctgaacttccagaaggcccggaagtccgccaacctggtcatttctgcttctcagctgggcgacagcgccatgtacttttgtgccatgagcggcgatagcgccggcaacatgcttacatttggcggcggaacccggctgatggtcaagccccatattcagaaccccgatcctgccgtgtaccagctgagagacagcaagagcagcgacaagagcgtgtgtctgttcaccgacttcgacagccagaccaacgtgtcccagagcaaggacagcgacgtgtacatcaccgacaagaccgtgctggacatgcggagcatggacttcaagagcaacagcgccgtggcctggtccaacaagagcgatttcgcctgcgccaacgccttcaacaatagcattatccccgaggacacattcttccccagctccgatgtgccctgcgacgtgaagctggtggaaaagagcttcgagacagacaccaacctgaacttccagaacctgagcgtgatcggcttcagaatcctgctgctgaaggtggccggcttcaatctgctgatgaccctgagactgtggtccagctga

SEQ ID NO: 39 - DNA, 939 bp, synthetic construct
  misc_feature 1..939: note = with minimal murinized constant region; codon optimized
  source 1..939: mol_type = other DNA
atggccagcctgctgttcttctgcggcgccttttatctgctcggcaccggctctatggacgccgacgttacacagacccctcggaacagaatcaccaagaccggcaagcggatcatgctggaatgcagccagaccaagggccacgaccggatgtactggtacagacaggaccctggcctgggcctgagactgatctactacagcttcgacgtgaaggacatcaacaagggcgagatcagcgacggctacagcgtgtcaagacaggctcaggccaagttcagcctgtctctggaaagcgctatccccaaccagacagccctgtacttctgcgccaccagcgactgggatagaagcggcgacaaagagacacagtacttcggccctggcaccagactgctggtgctggaagatctgaacaaggtgttccctccagaggtggccgtgttcgagccttctaaggccgagattgcccacacacagaaagccacactcgtgtgcctggctaccggcttctttcctgaccacgtggaactgtcttggtgggtcaacggcaaagaggtgcacagcggcgtcagcacagatccccagcctctgaaagaacagcccgctctgaacgacagccggtactgtctgagcagcagactgagagtgtccgccacattctggcagaaccccagaaaccacttcagatgccaggtgcagttctacggcctgagcgagaacgatgagtggacccaggatagagccaagcctgtgacacagatcgtgtctgccgaagcctggggcagagccgattgtggaattaccagcgccagctaccatcagggcgtgctgtctgccacaatcctgtacgagatcctgctgggcaaagccactctgtacgccgtgctggtgtctgccctggtgctgatggccatggtcaagagaaaggacttttga

SEQ ID NO: 40 - DNA, 21 bp, synthetic construct
  misc_feature 1..21: note = codon optimized
  source 1..21: mol_type = other DNA
accagcgatcctagctacggc

SEQ ID NO: 41 - DNA, 24 bp, synthetic construct
  misc_feature 1..24: note = codon optimized
  source 1..24: mol_type = other DNA
cagggcagctacgaccagcagaat

SEQ ID NO: 42 - DNA, 45 bp, synthetic construct
  misc_feature 1..45: note = codon optimized
  source 1..45: mol_type = other DNA
tgtgccatgagcggcggctacaccggcggcttcaagacaatcttt

SEQ ID NO: 43 - DNA, 15 bp, synthetic construct
  misc_feature 1..15: note = codon optimized
  source 1..15: mol_type = other DNA
agcggcgacctgagc

SEQ ID NO: 44 - DNA, 18 bp, synthetic construct
  misc_feature 1..18: note = codon optimized
  source 1..18: mol_type = other DNA
tactacaacggcgaggaa

SEQ ID NO: 45 - DNA, 39 bp, synthetic construct
  misc_feature 1..39: note = codon optimized
  source 1..39: mol_type = other DNA
tgtgccagctctggcggagatggcgacgagcagtttttt

SEQ ID NO: 46 - DNA, 834 bp, synthetic construct
  misc_feature 1..834: note = with minimal murinized constant region; codon optimized
  source 1..834: mol_type = other DNA
atgagcctgagcagcctgctgaaggtcgtgacagcctctctgtggctcggacctggaatcgcccagaagatcacccagacacagcccggcatgttcgtgcaagagaaagaagccgtgacactggactgcacctacgacaccagcgatcctagctacggcctgttctggtacaagcagcctagcagcggcgagatgatcttcctgatctaccagggcagctacgaccagcagaatgccaccgagggcagatacagcctgaacttccagaaggcccggaagtccgccaacctggtcatttctgctagccagctgggcgacagcgccatgtacttttgtgccatgagcggcggctacaccggcggcttcaagacaatctttggcgccggaaccagactgttcgtgaaggccaatattcagaaccccgatcctgccgtgtaccagctgagagacagcaagagcagcgacaagagcgtgtgtctgttcaccgacttcgacagccagaccaacgtgtcccagagcaaggacagcgacgtgtacatcaccgacaagaccgtgctggacatgcggagcatggacttcaagagcaacagcgccgtggcctggtccaacaagagcgatttcgcctgcgccaacgccttcaacaatagcattatccccgaggacacattcttccccagctccgatgtgccctgcgacgtgaagctggtggaaaagagcttcgagacagacaccaacctgaacttccagaacctgagcgtgatcggcttcagaatcctgctgctgaaggtggccggcttcaatctgctgatgaccctgagactgtggtccagctga

SEQ ID NO: 47 - DNA, 933 bp, synthetic construct
  misc_feature 1..933: note = with minimal murinized constant region; codon optimized
  source 1..933: mol_type = other DNA
atgggcttcagactgctgtgctgcgtggccttttgtctgcttggagccggacctgtggatagcggcgttacccagacacctaagcacctgatcacagccacaggccagcgcgtgaccctgagatgttctcctagaagcggcgacctgagcgtgtactggtatcagcagtctctggaccagggcctgcagttcctgatccagtactacaacggcgaggaaagagccaagggcaacatcctggaacggttcagcgcccagcagttcccagatctgcacagcgagctgaacctgagcagcctggaactgggagatagcgccctgtacttctgtgccagctctggcggagatggcgacgagcagttttttggccctggcaccagactgaccgtgctggaggacctgaagaacgtgttccctccggaggtggccgtgttcgagcccagcaaagccgagatcgcgcacacccagaaggccaccctggtgtgcctggccaccggcttctaccccgaccacgtggagctgagctggtgggtgaacggcaaggaggtgcacagcggcgtgagcaccgacccccagcccctgaaggagcagcccgccctgaacgacagccgctactgcctgagcagccgcctgcgcgtgagcgccaccttctggcagaacccccgcaaccacttccgctgccaggtgcagttctacggcctgagcgagaacgacgagtggacccaggaccgcgccaagcccgtgacccagatcgtgagcgccgaggcctggggccgcgccgactgcggcattaccagccgcagctaccatcagggcgtgctgagcgccaccatcctgtacgagatcctgctgggcaaggccaccctgtacgccgtgctggtgagcgccctggtgctgatggcgatggtgaagcgcaaggacagccgcggctga

SEQ ID NO: 48 - DNA, 18 bp, synthetic construct
  misc_feature 1..18: note = codon optimized
  source 1..18: mol_type = other DNA
gacagcgccagcaactac

SEQ ID NO: 49 - DNA, 21 bp, synthetic construct
  misc_feature 1..21: note = codon optimized
  source 1..21: mol_type = other DNA
atccggtccaacgtgggcgag

SEQ ID NO: 50 - DNA, 42 bp, synthetic construct
  misc_feature 1..42: note = codon optimized
  source 1..42: mol_type = other DNA
tgcgctgccagcagaggcaccggcttccagaaactggtgttt

SEQ ID NO: 51 - DNA, 15 bp, synthetic construct
  misc_feature 1..15: note = codon optimized
  source 1..15: mol_type = other DNA
ctgggccacgacacc

SEQ ID NO: 52 - DNA, 18 bp, synthetic construct
  misc_feature 1..18: note = codon optimized
  source 1..18: mol_type = other DNA
tacaacaacaaagagctg

SEQ ID NO: 53 - DNA, 48 bp, synthetic construct
  misc_feature 1..48: note = codon optimized
  source 1..48: mol_type = other DNA
tgtgccagcagccagttctgggatggcgctggcgacgagcagtatttt

SEQ ID NO: 54 - DNA, 822 bp, synthetic construct
  misc_feature 1..822: note = with minimal murinized constant region; codon optimized
  source 1..822: mol_type = other DNA
atgaccagcatccgggccgtgttcatcttcctgtggctgcagctggacctggtcaacggcgagaatgtggaacagcaccccagcacactgagcgtgcaagagggcgattctgccgtgatcaagtgcacctacagcgacagcgccagcaactacttcccctggtacaagcaagagctgggcaaaagaccccagctgatcatcgacatccggtccaacgtgggcgagaagaaggaccagagaatcgccgtgacactgaacaagaccgccaagcacttcagcctgcacatcaccgagacacagcctgaggatagcgccgtgtacttttgcgctgccagcagaggcaccggcttccagaaactggtgtttggcaccggcaccagactgctggtgtccccaaatattcagaaccccgatcctgccgtgtaccagctgagagacagcaagagcagcgacaagagcgtgtgtctgttcaccgacttcgacagccagaccaacgtgtcccagagcaaggacagcgacgtgtacatcaccgacaagaccgtgctggacatgcggagcatggacttcaagagcaacagcgccgtggcctggtccaacaagagcgatttcgcctgcgccaacgccttcaacaatagcattatccccgaggacacattcttccccagctccgatgtgccctgcgacgtgaagctggtggaaaagagcttcgagacagacaccaacctgaacttccagaacctgagcgtgatcggcttcagaatcctgctgctgaaggtggccggcttcaatctgctgatgaccctgagactgtggtccagctga

SEQ ID NO: 55 - DNA, 936 bp, synthetic construct
  misc_feature 1..936: note = with minimal murinized constant region; codon optimized
  source 1..936: mol_type = other DNA
atgggctgcagactgctgtgctgtgtggtgttctgcctgctgcaagccggacctctggatacagccgtgtctcagacccctaagtacctggtcacccagatgggcaacgacaagagcatcaagtgcgagcagaacctgggccacgacaccatgtactggtacaagcaggacagcaagaaattcctgaagatcatgttcagctacaacaacaaagagctgatcatcaacgagacagtgcccaacagattcagccctaagagccccgataaggcccacctgaacctgcacatcaacagcctggaactgggcgacagcgccgtgtacttttgtgccagcagccagttctgggatggcgctggcgacgagcagtattttggccctggcaccagactgaccgtgaccgaggacctgaacaaggtgttccctccggaggtggccgtgttcgagcccagcaaagccgagatcgcgcacacccagaaggccaccctggtgtgcctggccaccggcttcttccccgaccacgtggagctgagctggtgggtgaacggcaaggaggtgcacagcggcgtgagcaccgacccccagcccctgaaggagcagcccgccctgaacgacagccgctactgcctgagcagccgcctgcgcgtgagcgccaccttctggcagaacccccgcaaccacttccgctgccaggtgcagttctacggcctgagcgagaacgacgagtggacccaggaccgcgccaagcccgtgacccagatcgtgagcgccgaggcctggggccgcgccgactgcggcattaccagcgcgagctaccatcagggcgtgctgagcgccaccatcctgtacgagatcctgctgggcaaggccaccctgtacgccgtgctggtgagcgccctggtgctgatggcgatggtgaagcgcaaggacttctga

SEQ ID NO: 56 - protein, 10 aa, Homo sapiens
KLYGLDWAEL

SEQ ID NO: 57 - protein, 272 aa, synthetic construct
  REGION 1..272: note = murinized
MSLSSLLKVVTASLWLGPGIAQKITQTQPGMFVQEKEAVTLDCTYDTSDQSYGLFWYKQPSSGEMIFLIYQGSYDEQNATEGRYSLNFQKARKSANLVISASQLGDSAMYFCAMSGDSAGNMLTFGGGTRLMVKPHIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 58 - protein, 308 aa, synthetic construct
  REGION 1..308: note = murinized
MASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATSDWDRSGDKETQYFGPGTRLLVLEDLRNVTPPKVTLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 59 - protein, 273 aa, synthetic construct
  REGION 1..273: note = murinized
MSLSSLLKVVTASLWLGPGIAQKITQTQPGMFVQEKEAVTLDCTYDTSDPSYGLFWYKQPSSGEMIFLIYQGSYDQQNATEGRYSLNFQKARKSANLVISASQLGDSAMYFCAMSGGYTGGFKTIFGAGTRLFVKANIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 60 - protein, 304 aa, synthetic construct
  REGION 1..304: note = murinized
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSGGDGDEQFFGPGTRLTVLEDLRNVTPPKVTLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 61 - protein, 269 aa, synthetic construct
  REGION 1..269: note = murinized
MTSIRAVFIFLWLQLDLVNGENVEQHPSTLSVQEGDSAVIKCTYSDSASNYFPWYKQELGKRPQLIIDIRSNVGEKKDQRIAVTLNKTAKHFSLHITETQPEDSAVYFCAASRGTGFQKLVFGTGTRLLVSPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 62 - protein, 307 aa, synthetic construct
  REGION 1..307: note = murinized
MGCRLLCCVVFCLLQAGPLDTAVSQTPKYLVTQMGNDKSIKCEQNLGHDTMYWYKQDSKKFLKIMFSYNNKELIINETVPNRFSPKSPDKAHLNLHINSLELGDSAVYFCASSQFWDGAGDEQYFGPGTRLTVTEDLRNVTPPKVTLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 63 - DNA, 21 bp, synthetic construct
  misc_feature 1..21: note = human optimized
  source 1..21: mol_type = other DNA
acttcagaccagtcgtacggt

SEQ ID NO: 64 - DNA, 24 bp, synthetic construct
  misc_feature 1..24: note = human optimized
  source 1..24: mol_type = other DNA
cagggatcgtacgacgagcagaac

SEQ ID NO: 65 - DNA, 42 bp, synthetic construct
  misc_feature 1..42: note = human optimized
  source 1..42: mol_type = other DNA
tgcgcaatgtccggcgatagcgcaggaaacatgctgactttc

SEQ ID NO: 66 - DNA, 15 bp, synthetic construct
  misc_feature 1..15: note = human optimized
  source 1..15: mol_type = other DNA
aagggccacgaccgc

SEQ ID NO: 67 - DNA, 18 bp, synthetic construct
  misc_feature 1..18: note = human optimized
  source 1..18: mol_type = other DNA
tccttcgacgtgaaggac

SEQ ID NO: 68 - DNA, 51 bp, synthetic construct
  misc_feature 1..51: note = human optimized
  source 1..51: mol_type = other DNA
tgtgccacctcggattgggaccgatccggcgacaaggaaactcagtacttc

SEQ ID NO: 69 - DNA, 831 bp, synthetic construct
  misc_feature 1..831: note = human optimized
  source 1..831: mol_type = other DNA
atgagcctctcttccctgctcaaagtggtcactgcctccctgtggctgggaccgggaatcgcccagaagatcactcagacccagcctggaatgttcgtgcaagagaaagaggccgtgaccctggattgtacttatgacacttcagaccagtcgtacggtttgttctggtacaagcagccgtcctccggagaaatgatcttcctgatctaccagggatcgtacgacgagcagaacgctaccgagggcagatattccctcaacttccaaaaggcccggaaatccgcgaacctcgtgatctcggcctcacaacttggggactccgctatgtatttctgcgcaatgtccggcgatagcgcaggaaacatgctgactttcggcggtggaactaggctgatggtcaagccccacattcaaaaccctgacccagcagtctaccagttgcgggattccaagtcttccgataaatccgtgtgtctctttacagacttcgatagccagaccaacgtgtcccagagcaaagacagcgacgtgtacattactgacaagactgtgctggacatgcggtccatggacttcaagagcaactccgccgtcgcttggtccaacaagtctgactttgcgtgcgcgaacgctttcaacaacagcattatcccggaggacacctttttcccttcccccgagtcaagctgcgatgtcaagcttgtggaaaagtcgttcgaaaccgacaccaacctgaacttccagaacctgtccgtcatcgggttccgcattctgctgctgaaggtcgccggcttcaatctcctgatgactctccgcttgtggtcctcataa

SEQ ID NO: 70 - DNA, 936 bp, synthetic construct
  misc_feature 1..936: note = human optimized
  source 1..936: mol_type = other DNA
atggcgtccctgctgttcttctgcggtgccttctaccttctgggaaccggctcgatggacgccgacgtgacccaaacccctcgcaaccgcatcaccaagactggaaagcggatcatgctggaatgctcccagaccaagggccacgaccgcatgtactggtacagacaggacccgggtctgggattgcgcctgatctactactccttcgacgtgaaggacatcaacaagggggagatctccgatggatactcagtctcgagacaagcccaggctaagttttccctgtccctcgaatccgccattcccaatcagaccgcgctgtacttctgtgccacctcggattgggaccgatccggcgacaaggaaactcagtacttcggaccaggaaccaggctcctggtgctggaggatctgaacaaggtgttcccgccggaagtggcagtgttcgagccatccgaagccgagatctcgcatacgcagaaggccaccctcgtgtgcctggccactgggtttttccctgaccacgtggagctctcgtggtgggtcaacggaaaggaagtgcacagcggtgtctcaaccgacccgcaacctctcaaggaacagcccgcgctcaatgattcgcggtactgcctgagcagccggctcagagtgtccgccactttctggcaaaacccgcggaaccatttccggtgccaagtgcaattctacgggctgtcggaaaacgacgaatggacccaggacagggccaagcccgtgacccagattgtgtccgctgaagcctgggggagagctgattgcggtttcaccagcgtgtcgtatcagcagggcgtgctgtcagccaccattctgtacgaaatcctcctggggaaggccacactgtacgccgtgctcgtgtccgccctggtgctgatggccatggtcaagcggaaggacttc

SEQ ID NO: 71 - DNA, 21 bp, synthetic construct
  misc_feature 1..21: note = human optimized
  source 1..21: mol_type = other DNA
acctccgacccgtcctacggg

SEQ ID NO: 72 - DNA, 24 bp, synthetic construct
  misc_feature 1..24: note = human optimized
  source 1..24: mol_type = other DNA
caaggctcctatgatcagcagaac

SEQ ID NO: 73 - DNA, 45 bp, synthetic construct
  misc_feature 1..45: note = human optimized
  source 1..45: mol_type = other DNA
tgtgccatgtcgggcggatacaccgggggattcaagaccattttc

SEQ ID NO: 74 - DNA, 15 bp, synthetic construct
  misc_feature 1..15: note = human optimized
  source 1..15: mol_type = other DNA
tctggcgacctctcc

SEQ ID NO: 75 - DNA, 18 bp, synthetic construct
  misc_feature 1..18: note = human optimized
  source 1..18: mol_type = other DNA
tactacaacggagaggag

SEQ ID NO: 76 - DNA, 39 bp, synthetic construct
  misc_feature 1..39: note = human optimized
  source 1..39: mol_type = other DNA
tgcgcaagcagcggtggtgacggggatgaacagttcttt

SEQ ID NO: 77 - DNA, 834 bp, synthetic construct
  misc_feature 1..834: note = human optimized
  source 1..834: mol_type = other DNA
atgtcccttagctcactgctgaaagtggtcactgcgagcctgtggctgggaccaggcattgctcaaaagatcacccagactcagcctgggatgttcgtgcaagagaaggaagccgtgaccctcgactgcacttacgacacctccgacccgtcctacgggctgttctggtacaagcagccgtcctccggagagatgatcttcctcatctaccaaggctcctatgatcagcagaacgccaccgaaggacgctacagcctgaacttccagaaggctcggaagtcggcgaacctcgtgatcagcgcatcccaactgggggacagcgccatgtacttctgtgccatgtcgggcggatacaccgggggattcaagaccattttcggggccggcactagactgttcgtgaaggccaacatccagaatcctgatccggcggtgtatcagctgcgcgactccaagtcttccgataaatccgtgtgtctctttacagacttcgactcccaaaccaacgtgtcacagtccaaggacagcgatgtgtacatcaccgacaaaaccgtgctggacatgcggtccatggacttcaagtcaaacagcgcagtcgcctggtccaacaagtccgacttcgcctgtgcgaacgccttcaacaactccatcattccggaagatacctttttcccttcacctgagtcgagctgtgatgtgaagctcgtggaaaagtcgtttgaaacggacaccaacctgaactttcagaacctgtccgtgattggtttccgcatcctgctgctgaaggtcgccggcttcaacttgctgatgacgctccggctgtggtcctcgtaa

SEQ ID NO: 78 - DNA, 930 bp, synthetic construct
  misc_feature 1..930: note = human optimized
  source 1..930: mol_type = other DNA
atgggattccgccttctgtgctgcgtggccttctgcttgcttggagctggtcccgtcgactcgggagtgacccagacgccgaagcacttgattaccgctactgggcagcgcgtgactctgcgatgctcaccacggtctggcgacctctccgtgtactggtatcagcagagcctggaccagggactgcagttcctgatccagtactacaacggagaggagcgggctaagggaaacatactggagcggttctcggcgcaacaattccccgatctgcactccgaactgaacctgtcctccctggaattgggagactccgccctgtacttctgcgcaagcagcggtggtgacggggatgaacagttctttggccctggaaccagactcaccgtgctcgaggacctcaagaacgtgttcccacccgaagtcgcggtgttcgagccctccgaagcggaaatcagccatactcagaaagccactctcgtgtgcctggccaccggattctacccggaccacgtcgagctctcttggtgggtgaacgggaaagaggtccacagcggcgtgagcactgatccgcagccgctgaaggaacaacccgccttgaacgactcgcggtactgtctgtcctcccggctgagagtgtcggccaccttctggcaaaaccccaggaaccactttaggtgccaagtccagttctacggcctgagcgaaaacgatgagtggacccaggacagagccaagcctgtgacccagattgtgtcagccgaggcttggggtagagcagactgcggattcacctccgagtcctaccaacaaggcgtcctctcggcgaccattctgtacgaaatcctgcttgggaaggccactctgtacgccgtgctggtgtccgccctggtgctgatggccatggtcaagcggaaggactcccggggg

SEQ ID NO: 79 - DNA, 18 bp, synthetic construct
  misc_feature 1..18: note = human optimized
  source 1..18: mol_type = other DNA
gactcagcatcaaactat

SEQ ID NO: 80 - DNA, 21 bp, synthetic construct
  misc_feature 1..21: note = human optimized
  source 1..21: mol_type = other DNA
atccgctccaacgtcggagag

SEQ ID NO: 81 - DNA, 42 bp, synthetic construct
  misc_feature 1..42: note = human optimized
  source 1..42: mol_type = other DNA
tgtgcggcctcgaggggcactgggtttcagaagctcgtgttc

SEQ ID NO: 82 - DNA, 15 bp, synthetic construct
  misc_feature 1..15: note = human optimized
  source 1..15: mol_type = other DNA
ctcggacacgacacc

SEQ ID NO: 83 - DNA, 18 bp, synthetic construct
  misc_feature 1..18: note = human optimized
  source 1..18: mol_type = other DNA
tacaacaacaaggaactg

SEQ ID NO: 84 - DNA, 48 bp, synthetic construct
  misc_feature 1..48: note = human optimized
  source 1..48: mol_type = other DNA
tgcgcgtccagccagttctgggacggagcgggcgacgaacagtacttc

SEQ ID NO: 85 - DNA, 819 bp, synthetic construct
  misc_feature 1..819: note = human optimized
  source 1..819: mol_type = other DNA
atgacgtccattagagccgtgttcattttcctgtggctgcaactggacctcgtgaatggagaaaacgtcgagcagcacccatccaccctgagcgtgcaggagggagactccgcagtcatcaagtgcacttactccgactcagcatcaaactatttcccgtggtataagcaggaactcggaaagcggcctcagctcatcattgacatccgctccaacgtcggagagaagaaggaccagagaattgccgtgacactcaacaagaccgccaagcatttctcccttcacatcaccgaaacccagcccgaggacagcgccgtctacttttgtgcggcctcgaggggcactgggtttcagaagctcgtgttcggcactgggacccggctgctggtgtcgccaaacatccagaatccagaccccgcggtgtaccagctgagagactcgaagtcttccgataaatccgtgtgtctctttacagacttcgatagccagactaacgtgtcccagtccaaggactccgatgtgtacatcaccgacaagactgtgctggatatgcggagcatggactttaagtccaattcagcggtcgcgtggagcaacaagtccgacttcgcctgcgctaacgctttcaacaactccattatcccggaggataccttcttcccgtcaccggaatcctcgtgcgacgtgaagctggtcgagaagtccttcgaaaccgataccaacctgaacttccaaaacctctccgtgatcggcttcagaatcctgctgctgaaagtggctggcttcaatttgctgatgaccctgcggctctggagcagc

SEQ ID NO: 86 - DNA, 930 bp, synthetic construct
  misc_feature 1..930: note = human optimized
  source 1..930: mol_type = other DNA
atgggttgtcggttgctgtgttgcgtcgtgttctgccttcttcaagctggtcctctcgatactgccgtgagccaaacccctaagtaccttgtcacccaaatgggcaacgacaagtccatcaaatgcgaacagaacctcggacacgacaccatgtactggtacaaacaggattccaagaagttcctgaagattatgttctcatacaacaacaaggaactgattatcaacgaaactgtgccgaaccggttctcaccgaagtcgcctgacaaggctcatctcaacttgcatatcaactcgctggagctcggcgactccgccgtgtacttctgcgcgtccagccagttctgggacggagcgggcgacgaacagtacttcggcccgggcactcggctgaccgtgaccgaagatctgaacaaagtgttcccccccgaagtggccgtgttcgaaccttccgaggccgagatcagccacacccaaaaggccactctggtctgcctggccaccggtttcttccccgatcacgtggaactgtcttggtgggtgaacggaaaagaagtgcactcgggggtgtccacggacccccagcctctgaaggaacagccggcactgaatgactcacgctactgtctgtcgtcacggctgcgcgtgtcggccaccttctggcaaaacccgcgaaaccactttcgctgccaagtgcagttttacgggctttccgagaacgacgagtggactcaggacagagcgaagcccgtgacccaaatcgtgtccgccgaggcctggggacgcgccgactgcggtttcacctccgtgagctaccaacagggcgtgctgtcagctaccatcctttacgagatcctcctgggaaaggccaccctctacgccgtcctggtgtccgcactggtgctgatggctatggtcaagcggaaggat

SEQ ID NO: 87 - protein, 276 aa, Homo sapiens
MSLSSLLKVVTASLWLGPGIAQKITQTQPGMFVQEKEAVTLDCTYDTSDQSYGLFWYKQPSSGEMIFLIYQGSYDEQNATEGRYSLNFQKARKSANLVISASQLGDSAMYFCAMSGDSAGNMLTFGGGTRLMVKPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 88 - protein, 312 aa, Homo sapiens
MASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATSDWDRSGDKETQYFGPGTRLLVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 89 - protein, 277 aa, Homo sapiens
MSLSSLLKVVTASLWLGPGIAQKITQTQPGMFVQEKEAVTLDCTYDTSDPSYGLFWYKQPSSGEMIFLIYQGSYDQQNATEGRYSLNFQKARKSANLVISASQLGDSAMYFCAMSGGYTGGFKTIFGAGTRLFVKANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 90 - protein, 310 aa, Homo sapiens
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSGGDGDEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 91 - protein, 273 aa, Homo sapiens
MTSIRAVFIFLWLQLDLVNGENVEQHPSTLSVQEGDSAVIKCTYSDSASNYFPWYKQELGKRPQLIIDIRSNVGEKKDQRIAVTLNKTAKHFSLHITETQPEDSAVYFCAASRGTGFQKLVFGTGTRLLVSPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 92 - protein, 310 aa, Homo sapiens
MGCRLLCCVVFCLLQAGPLDTAVSQTPKYLVTQMGNDKSIKCEQNLGHDTMYWYKQDSKKFLKIMFSYNNKELIINETVPNRFSPKSPDKAHLNLHINSLELGDSAVYFCASSQFWDGAGDEQYFGPGTRLTVTEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKD

SEQ ID NO: 93 - DNA, 1833 bp, synthetic construct
  misc_feature 1..1833: note = human optimized
  source 1..1833: mol_type = other DNA
atggcgtccctgctgttcttctgcggtgccttctaccttctgggaaccggctcgatggacgccgacgtgacccaaacccctcgcaaccgcatcaccaagactggaaagcggatcatgctggaatgctcccagaccaagggccacgaccgcatgtactggtacagacaggacccgggtctgggattgcgcctgatctactactccttcgacgtgaaggacatcaacaagggggagatctccgatggatactcagtctcgagacaagcccaggctaagttttccctgtccctcgaatccgccattcccaatcagaccgcgctgtacttctgtgccacctcggattgggaccgatccggcgacaaggaaactcagtacttcggaccaggaaccaggctcctggtgctggaggatctgaacaaggtgttcccgccggaagtggcagtgttcgagccatccgaagccgagatctcgcatacgcagaaggccaccctcgtgtgcctggccactgggtttttccctgaccacgtggagctctcgtggtgggtcaacggaaaggaagtgcacagcggtgtctcaaccgacccgcaacctctcaaggaacagcccgcgctcaatgattcgcggtactgcctgagcagccggctcagagtgtccgccactttctggcaaaacccgcggaaccatttccggtgccaagtgcaattctacgggctgtcggaaaacgacgaatggacccaggacagggccaagcccgtgacccagattgtgtccgctgaagcctgggggagagctgattgcggtttcaccagcgtgtcgtatcagcagggcgtgctgtcagccaccattctgtacgaaatcctcctggggaaggccacactgtacgccgtgctcgtgtccgccctggtgctgatggccatggtcaagcggaaggacttcggcagcggagctaccaacttctccctgctgaagcaggccggcgatgtggaagaaaatcccggacctatgagcctctcttccctgctcaaagtggtcactgcctccctgtggctgggaccgggaatcgcccagaagatcactcagacccagcctggaatgttcgtgcaagagaaagaggccgtgaccctggattgtacttatgacacttcagaccagtcgtacggtttgttctggtacaagcagccgtcctccggagaaatgatcttcctgatctaccagggatcgtacgacgagcagaacgctaccgagggcagatattccctcaacttccaaaaggcccggaaatccgcgaacctcgtgatctcggcctcacaacttggggactccgctatgtatttctgcgcaatgtccggcgatagcgcaggaaacatgctgactttcggcggtggaactaggctgatggtcaagccccacattcaaaaccctgacccagcagtctaccagttgcgggattccaagtcttccgataaatccgtgtgtctctttacagacttcgatagccagaccaacgtgtcccagagcaaagacagcgacgtgtacattactgacaagactgtgctggacatgcggtccatggacttcaagagcaactccgccgtcgcttggtccaacaagtctgactttgcgtgcgcgaacgctttcaacaacagcattatcccggaggacacctttttcccttcccccgagtcaagctgcgatgtcaagcttgtggaaaagtcgttcgaaaccgacaccaacctgaacttccagaacctgtccgtcatcgggttccgcattctgctgctgaaggtcgccggcttcaatctcctgatgactctccgcttgtggtcctcataa

SEQ ID NO: 94 - protein, 610 aa, synthetic construct
  REGION 1..610: note = human optimized
MASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATSDWDRSGDKETQYFGPGTRLLVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGATNFSLLKQAGDVEENPGPMSLSSLLKVVTASLWLGPGIAQKITQTQPGMFVQEKEAVTLDCTYDTSDQSYGLFWYKQPSSGEMIFLIYQGSYDEQNATEGRYSLNFQKARKSANLVISASQLGDSAMYFCAMSGDSAGNMLTFGGGTRLMVKPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 95 - DNA, 1842 bp, synthetic construct
  misc_feature 1..1842: note = human optimized
  source 1..1842: mol_type = other DNA
atgggattccgccttctgtgctgcgtggccttctgcttgcttggagctggtcccgtcgactcgggagtgacccagacgccgaagcacttgattaccgctactgggcagcgcgtgactctgcgatgctcaccacggtctggcgacctctccgtgtactggtatcagcagagcctggaccagggactgcagttcctgatccagtactacaacggagaggagcgggctaagggaaacatactggagcggttctcggcgcaacaattccccgatctgcactccgaactgaacctgtcctccctggaattgggagactccgccctgtacttctgcgcaagcagcggtggtgacggggatgaacagttctttggccctggaaccagactcaccgtgctcgaggacctcaagaacgtgttcccacccgaagtcgcggtgttcgagccctccgaagcggaaatcagccatactcagaaagccactctcgtgtgcctggccaccggattctacccggaccacgtcgagctctcttggtgggtgaacgggaaagaggtccacagcggcgtgagcactgatccgcagccgctgaaggaacaacccgccttgaacgactcgcggtactgtctgtcctcccggctgagagtgtcggccaccttctggcaaaaccccaggaaccactttaggtgccaagtccagttctacggcctgagcgaaaacgatgagtggacccaggacagagccaagcctgtgacccagattgtgtcagccgaggcttggggtagagcagactgcggattcacctccgagtcctaccaacaaggcgtcctctcggcgaccattctgtacgaaatcctgcttgggaaggccactctgtacgccgtgctggtgtccgccctggtgctgatggccatggtcaagcggaaggactcccgggggagagcaaagaggggatcgggagccaccaattttagcctgctgaagcaggccggcgatgtggaagaaaatcctggccccatgtcccttagctcactgctgaaagtggtcactgcgagcctgtggctgggaccaggcattgctcaaaagatcacccagactcagcctgggatgttcgtgcaagagaaggaagccgtgaccctcgactgcacttacgacacctccgacccgtcctacgggctgttctggtacaagcagccgtcctccggagagatgatcttcctcatctaccaaggctcctatgatcagcagaacgccaccgaaggacgctacagcctgaacttccagaaggctcggaagtcggcgaacctcgtgatcagcgcatcccaactgggggacagcgccatgtacttctgtgccatgtcgggcggatacaccgggggattcaagaccattttcggggccggcactagactgttcgtgaaggccaacatccagaatcctgatccggcggtgtatcagctgcgcgactccaagtcttccgataaatccgtgtgtctctttacagacttcgactcccaaaccaacgtgtcacagtccaaggacagcgatgtgtacatcaccgacaaaaccgtgctggacatgcggtccatggacttcaagtcaaacagcgcagtcgcctggtccaacaagtccgacttcgcctgtgcgaacgccttcaacaactccatcattccggaagatacctttttcccttcacctgagtcgagctgtgatgtgaagctcgtggaaaagtcgtttgaaacggacaccaacctgaactttcagaacctgtccgtgattggtttccgcatcctgctgctgaaggtcgccggcttcaacttgctgatgacgctccggctgtggtcctcgtaa

SEQ ID NO: 96 - protein, 613 aa, synthetic construct
  REGION 1..613: note = human optimized
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSGGDGDEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRAKRGSGATNFSLLKQAGDVEENPGPMSLSSLLKVVTASLWLGPGIAQKITQTQPGMFVQEKEAVTLDCTYDTSDPSYGLFWYKQPSSGEMIFLIYQGSYDQQNATEGRYSLNFQKARKSANLVISASQLGDSAMYFCAMSGGYTGGFKTIFGAGTRLFVKANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 97 - DNA, 1830 bp, synthetic construct
  misc_feature 1..1830: note = human optimized
  source 1..1830: mol_type = other DNA
atgggttgtcggttgctgtgttgcgtcgtgttctgccttcttcaagctggtcctctcgatactgccgtgagccaaacccctaagtaccttgtcacccaaatgggcaacgacaagtccatcaaatgcgaacagaacctcggacacgacaccatgtactggtacaaacaggattccaagaagttcctgaagattatgttctcatacaacaacaaggaactgattatcaacgaaactgtgccgaaccggttctcaccgaagtcgcctgacaaggctcatctcaacttgcatatcaactcgctggagctcggcgactccgccgtgtacttctgcgcgtccagccagttctgggacggagcgggcgacgaacagtacttcggcccgggcactcggctgaccgtgaccgaagatctgaacaaagtgttcccccccgaagtggccgtgttcgaaccttccgaggccgagatcagccacacccaaaaggccactctggtctgcctggccaccggtttcttccccgatcacgtggaactgtcttggtgggtgaacggaaaagaagtgcactcgggggtgtccacggacccccagcctctgaaggaacagccggcactgaatgactcacgctactgtctgtcgtcacggctgcgcgtgtcggccaccttctggcaaaacccgcgaaaccactttcgctgccaagtgcagttttacgggctttccgagaacgacgagtggactcaggacagagcgaagcccgtgacccaaatcgtgtccgccgaggcctggggacgcgccgactgcggtttcacctccgtgagctaccaacagggcgtgctgtcagctaccatcctttacgagatcctcctgggaaaggccaccctctacgccgtcctggtgtccgcactggtgctgatggctatggtcaagcggaaggattttagggccaaacgcgggtccggagcgaccaacttctcgctgttgaagcaggccggcgatgtggaagagaaccctggaccgatgacgtccattagagccgtgttcattttcctgtggctgcaactggacctcgtgaatggagaaaacgtcgagcagcacccatccaccctgagcgtgcaggagggagactccgcagtcatcaagtgcacttactccgactcagcatcaaactatttcccgtggtataagcaggaactcggaaagcggcctcagctcatcattgacatccgctccaacgtcggagagaagaaggaccagagaattgccgtgacactcaacaagaccgccaagcatttctcccttcacatcaccgaaacccagcccgaggacagcgccgtctacttttgtgcggcctcgaggggcactgggtttcagaagctcgtgttcggcactgggacccggctgctggtgtcgccaaacatccagaatccagaccccgcggtgtaccagctgagagactcgaagtcttccgataaatccgtgtgtctctttacagacttcgatagccagactaacgtgtcccagtccaaggactccgatgtgtacatcaccgacaagactgtgctggatatgcggagcatggactttaagtccaattcagcggtcgcgtggagcaacaagtccgacttcgcctgcgctaacgctttcaacaactccattatcccggaggataccttcttcccgtcaccggaatcctcgtgcgacgtgaagctggtcgagaagtccttcgaaaccgataccaacctgaacttccaaaacctctccgtgatcggcttcagaatcctgctgctgaaagtggctggcttcaatttgctgatgaccctgcggctctggagcagc

SEQ ID NO: 98 - protein, 610 aa, synthetic construct
  REGION 1..610: note = human optimized
MGCRLLCCVVFCLLQAGPLDTAVSQTPKYLVTQMGNDKSIKCEQNLGHDTMYWYKQDSKKFLKIMFSYNNKELIINETVPNRFSPKSPDKAHLNLHINSLELGDSAVYFCASSQFWDGAGDEQYFGPGTRLTVTEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFRAKRGSGATNFSLLKQAGDVEENPGPMTSIRAVFIFLWLQLDLVNGENVEQHPSTLSVQEGDSAVIKCTYSDSASNYFPWYKQELGKRPQLIIDIRSNVGEKKDQRIAVTLNKTAKHFSLHITETQPEDSAVYFCAASRGTGFQKLVFGTGTRLLVSPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 99 - DNA, 828 bp, synthetic construct
  misc_feature 1..828: note = TCR-7 a complete
  source 1..828: mol_type = other DNA
atgagcctctcttccctgctcaaagtggtcactgcctccctgtggctgggaccgggaatcgcccagaagatcactcagacccagcctggaatgttcgtgcaagagaaagaggccgtgaccctggattgtacttatgacacttcagaccagtcgtacggtttgttctggtacaagcagccgtcctccggagaaatgatcttcctgatctaccagggatcgtacgacgagcagaacgctaccgagggcagatattccctcaacttccaaaaggcccggaaatccgcgaacctcgtgatctcggcctcacaacttggggactccgctatgtatttctgcgcaatgtccggcgatagcgcaggaaacatgctgactttcggcggtggaactaggctgatggtcaagccccacattcaaaaccctgacccagcagtctaccagttgcgggattccaagtcttccgataaatccgtgtgtctctttacagacttcgatagccagaccaacgtgtcccagagcaaagacagcgacgtgtacattactgacaagactgtgctggacatgcggtccatggacttcaagagcaactccgccgtcgcttggtccaacaagtctgactttgcgtgcgcgaacgctttcaacaacagcattatcccggaggacacctttttcccttccagcgacgtcccctgcgatgtcaagcttgtggaaaagtcgttcgaaaccgacaccaacctgaacttccagaacctgctagtgattgtgctccgcattctgctgctgaaggtcgccggcttcaatctcctgatgactctccgcttgtggtcctca

SEQ ID NO: 100 - DNA, 936 bp, synthetic construct
  misc_feature 1..936: note = TCR-7 b complete
  source 1..936: mol_type = other DNA
atggcgtccctgctgttcttctgcggtgccttctaccttctgggaaccggctcgatggacgccgacgtgacccaaacccctcgcaaccgcatcaccaagactggaaagcggatcatgctggaatgctcccagaccaagggccacgaccgcatgtactggtacagacaggacccgggtctgggattgcgcctgatctactactccttcgacgtgaaggacatcaacaagggggagatctccgatggatactcagtctcgagacaagcccaggctaagttttccctgtccctcgaatccgccattcccaatcagaccgcgctgtacttctgtgccacctcggattgggaccgatccggcgacaaggaaactcagtacttcggaccaggaaccaggctcctggtgctggaggatctgaacaaggtgttcccgccggaagtggcagtgttcgagccatccaaggcggagatcgcccatacgcagaaggccaccctcgtgtgcctggccactgggtttttccctgaccacgtggagctctcgtggtgggtcaacggaaaggaagtgcacagcggtgtctcaaccgacccgcaacctctcaaggaacagcccgcgctcaatgattcgcggtactgcctgagcagccggctcagagtgtccgccactttctggcaaaacccgcggaaccatttccggtgccaagtgcaattctacgggctgtcggaaaacgacgaatggacccaggacagggccaagcccgtgacccagattgtgtccgctgaagcctgggggagagctgattgcggtatcaccagcgccagctaccaccagggcgtgctgtcagccaccattctgtacgaaatcctcctggggaaggccacactgtacgccgtgctcgtgtccgccctggtgctgatggccatggtcaagcggaaggacttc

SEQ ID NO: 101 - DNA, 1833 bp, synthetic construct
  misc_feature 1..1833: note = TCR-7 polycistronic construct
  source 1..1833: mol_type = other DNA
atggcgtccctgctgttcttctgcggtgccttctaccttctgggaaccggctcgatggacgccgacgtgacccaaacccctcgcaaccgcatcaccaagactggaaagcggatcatgctggaatgctcccagaccaagggccacgaccgcatgtactggtacagacaggacccgggtctgggattgcgcctgatctactactccttcgacgtgaaggacatcaacaagggggagatctccgatggatactcagtctcgagacaagcccaggctaagttttccctgtccctcgaatccgccattcccaatcagaccgcgctgtacttctgtgccacctcggattgggaccgatccggcgacaaggaaactcagtacttcggaccaggaaccaggctcctggtgctggaggatctgaacaaggtgttcccgccggaagtggcagtgttcgagccatccaaggcggagatcgcccatacgcagaaggccaccctcgtgtgcctggccactgggtttttccctgaccacgtggagctctcgtggtgggtcaacggaaaggaagtgcacagcggtgtctcaaccgacccgcaacctctcaaggaacagcccgcgctcaatgattcgcggtactgcctgagcagccggctcagagtgtccgccactttctggcaaaacccgcggaaccatttccggtgccaagtgcaattctacgggctgtcggaaaacgacgaatggacccaggacagggccaagcccgtgacccagattgtgtccgctgaagcctgggggagagctgattgcggtatcaccagcgccagctaccaccagggcgtgctgtcagccaccattctgtacgaaatcctcctggggaaggccacactgtacgccgtgctcgtgtccgccctggtgctgatggccatggtcaagcggaaggacttcggcagcggagctaccaacttctccctgctgaagcaggccggcgatgtggaagaaaatcccggacctatgagcctctcttccctgctcaaagtggtcactgcctccctgtggctgggaccgggaatcgcccagaagatcactcagacccagcctggaatgttcgtgcaagagaaagaggccgtgaccctggattgtacttatgacacttcagaccagtcgtacggtttgttctggtacaagcagccgtcctccggagaaatgatcttcctgatctaccagggatcgtacgacgagcagaacgctaccgagggcagatattccctcaacttccaaaaggcccggaaatccgcgaacctcgtgatctcggcctcacaacttggggactccgctatgtatttctgcgcaatgtccggcgatagcgcaggaaacatgctgactttcggcggtggaactaggctgatggtcaagccccacattcaaaaccctgacccagcagtctaccagttgcgggattccaagtcttccgataaatccgtgtgtctctttacagacttcgatagccagaccaacgtgtcccagagcaaagacagcgacgtgtacattactgacaagactgtgctggacatgcggtccatggacttcaagagcaactccgccgtcgcttggtccaacaagtctgactttgcgtgcgcgaacgctttcaacaacagcattatcccggaggacacctttttcccttccagcgacgtcccctgcgatgtcaagcttgtggaaaagtcgttcgaaaccgacaccaacctgaacttccagaacctgctagtgattgtgctccgcattctgctgctgaaggtcgccggcttcaatctcctgatgactctccgcttgtggtcctcataa

SEQ ID NO: 102 - protein, 276 aa, synthetic construct
  REGION 1..276: note = TCR-7 a complete
MSLSSLLKVVTASLWLGPGIAQKITQTQPGMFVQEKEAVTLDCTYDTSDQSYGLFWYKQPSSGEMIFLIYQGSYDEQNATEGRYSLNFQKARKSANLVISASQLGDSAMYFCAMSGDSAGNMLTFGGGTRLMVKPHIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWSS

SEQ ID NO: 103 - protein, 312 aa, synthetic construct; identical to SEQ ID NO: 11
  REGION 1..312: note = TCR-7 b complete

SEQ ID NO: 104 - protein, 610 aa, synthetic construct
  REGION 1..610: note = TCR-7 polyprotein
MASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATSDWDRSGDKETQYFGPGTRLLVLEDLNKVFPPEVAVFEPSKAEIAHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDFGSGATNFSLLKQAGDVEENPGPMSLSSLLKVVTASLWLGPGIAQKITQTQPGMFVQEKEAVTLDCTYDTSDQSYGLFWYKQPSSGEMIFLIYQGSYDEQNATEGRYSLNFQKARKSANLVISASQLGDSAMYFCAMSGDSAGNMLTFGGGTRLMVKPHIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWSS

SEQ ID NO: 105 - DNA, 831 bp, synthetic construct
  misc_feature 1..831: note = TCR-8 a complete
  source 1..831: mol_type = other DNA
atgtcccttagctcactgctgaaagtggtcactgcgagcctgtggctgggaccaggcattgctcaaaagatcacccagactcagcctgggatgttcgtgcaagagaaggaagccgtgaccctcgactgcacttacgacacctccgacccgtcctacgggctgttctggtacaagcagccgtcctccggagagatgatcttcctcatctaccaaggctcctatgatcagcagaacgccaccgaaggacgctacagcctgaacttccagaaggctcggaagtcggcgaacctcgtgatcagcgcatcccaactgggggacagcgccatgtacttctgtgccatgtcgggcggatacaccgggggattcaagaccattttcggggccggcactagactgttcgtgaaggccaacatccagaatcctgatccggcggtgtatcagctgcgcgactccaagtcttccgataaatccgtgtgtctctttacagacttcgactcccaaaccaacgtgtcacagtccaaggacagcgatgtgtacatcaccgacaaaaccgtgctggacatgcggtccatggacttcaagtcaaacagcgcagtcgcctggtccaacaagtccgacttcgcctgtgcgaacgccttcaacaactccatcattccggaagatacctttttcccttcaagcgacgtcccctgtgatgtgaagctcgtggaaaagtcgtttgaaacggacaccaacctgaactttcagaacctgctagtgattgtgctccgcatcctgctgctgaaggtcgccggcttcaacttgctgatgacgctccggctgtggtcctcg

SEQ ID NO: 106 - DNA, 930 bp, synthetic construct
  misc_feature 1..930: note = TCR-8 b complete
  source 1..930: mol_type = other DNA
atgggattccgccttctgtgctgcgtggccttctgcttgcttggagctggtcccgtcgactcgggagtgacccagacgccgaagcacttgattaccgctactgggcagcgcgtgactctgcgatgctcaccacggtctggcgacctctccgtgtactggtatcagcagagcctggaccagggactgcagttcctgatccagtactacaacggagaggagcgggctaagggaaacatactggagcggttctcggcgcaacaattccccgatctgcactccgaactgaacctgtcctccctggaattgggagactccgccctgtacttctgcgcaagcagcggtggtgacggggatgaacagttctttggccctggaaccagactcaccgtgctcgaggacctcaagaacgtgttcccacccgaagtcgcggtgttcgagccctccaaggcggagatcgcccatactcagaaagccactctcgtgtgcctggccaccggattctacccggaccacgtcgagctctcttggtgggtgaacgggaaagaggtccacagcggcgtgagcactgatccgcagccgctgaaggaacaacccgccttgaacgactcgcggtactgtctgtcctcccggctgagagtgtcggccaccttctggcaaaaccccaggaaccactttaggtgccaagtccagttctacggcctgagcgaaaacgatgagtggacccaggacagagccaagcctgtgacccagattgtgtcagccgaggcttggggtagagcagactgcggaatcaccagcgccagctaccaccaaggcgtcctctcggcgaccattctgtacgaaatcctgcttgggaaggccactctgtacgccgtgctggtgtccgccctggtgctgatggccatggtcaagcggaaggactcccggggg

SEQ ID NO: 107 - DNA, 1842 bp, synthetic construct
  misc_feature 1..1842: note = TCR-8 polycistronic construct
  source 1..1842: mol_type = other DNA
atgggattccgccttctgtgctgcgtggccttctgcttgcttggagctggtcccgtcgactcgggagtgacccagacgccgaagcacttgattaccgctactgggcagcgcgtgactctgcgatgctcaccacggtctggcgacctctccgtgtactggtatcagcagagcctggaccagggactgcagttcctgatccagtactacaacggagaggagcgggctaagggaaacatactggagcggttctcggcgcaacaattccccgatctgcactccgaactgaacctgtcctccctggaattgggagactccgccctgtacttctgcgcaagcagcggtggtgacggggatgaacagttctttggccctggaaccagactcaccgtgctcgaggacctcaagaacgtgttcccacccgaagtcgcggtgttcgagccctccaaggcggagatcgcccatactcagaaagccactctcgtgtgcctggccaccggattctacccggaccacgtcgagctctcttggtgggtgaacgggaaagaggtccacagcggcgtgagcactgatccgcagccgctgaaggaacaacccgccttgaacgactcgcggtactgtctgtcctcccggctgagagtgtcggccaccttctggcaaaaccccaggaaccactttaggtgccaagtccagttctacggcctgagcgaaaacgatgagtggacccaggacagagccaagcctgtgacccagattgtgtcagccgaggcttggggtagagcagactgcggaatcaccagcgccagctaccaccaaggcgtcctctcggcgaccattctgtacgaaatcctgcttgggaaggccactctgtacgccgtgctggtgtccgccctggtgctgatggccatggtcaagcggaaggactcccgggggagagcaaagaggggatcgggagccaccaattttagcctgctgaagcaggccggcgatgtggaagaaaatcctggccccatgtcccttagctcactgctgaaagtggtcactgcgagcctgtggctgggaccaggcattgctcaaaagatcacccagactcagcctgggatgttcgtgcaagagaaggaagccgtgaccctcgactgcacttacgacacctccgacccgtcctacgggctgttctggtacaagcagccgtcctccggagagatgatcttcctcatctaccaaggctcctatgatcagcagaacgccaccgaaggacgctacagcctgaacttccagaaggctcggaagtcggcgaacctcgtgatcagcgcatcccaactgggggacagcgccatgtacttctgtgccatgtcgggcggatacaccgggggattcaagaccattttcggggccggcactagactgttcgtgaaggccaacatccagaatcctgatccggcggtgtatcagctgcgcgactccaagtcttccgataaatccgtgtgtctctttacagacttcgactcccaaaccaacgtgtcacagtccaaggacagcgatgtgtacatcaccgacaaaaccgtgctggacatgcggtccatggacttcaagtcaaacagcgcagtcgcctggtccaacaagtccgacttcgcctgtgcgaacgccttcaacaactccatcattccggaagatacctttttcccttcaagcgacgtcccctgtgatgtgaagctcgtggaaaagtcgtttgaaacggacaccaacctgaactttcagaacctgctagtgattgtgctccgcatcctgctgctgaaggtcgccggcttcaacttgctgatgacgctccggctgtggtcctcgtaa

SEQ ID NO: 108 - protein, 277 aa, synthetic construct
  REGION 1..277: note = TCR-8 a complete
MSLSSLLKVVTASLWLGPGIAQKITQTQPGMFVQEKEAVTLDCTYDTSDPSYGLFWYKQPSSGEMIFLIYQGSYDQQNATEGRYSLNFQKARKSANLVISASQLGDSAMYFCAMSGGYTGGFKTIFGAGTRLFVKANIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWSS

SEQ ID NO: 109 - protein, 310 aa, synthetic construct
  REGION 1..310: note = TCR-8 b complete
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSGGDGDEQFFGPGTRLTVLEDLKNVFPPEVAVFEPSKAEIAHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDSRG

SEQ ID NO: 110 - protein, 613 aa, synthetic construct
  REGION 1..613: note = TCR-8 polyprotein
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSGGDGDEQFFGPGTRLTVLEDLKNVFPPEVAVFEPSKAEIAHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDSRGRAKRGSGATNFSLLKQAGDVEENPGPMSLSSLLKVVTASLWLGPGIAQKITQTQPGMFVQEKEAVTLDCTYDTSDPSYGLFWYKQPSSGEMIFLIYQGSYDQQNATEGRYSLNFQKARKSANLVISASQLGDSAMYFCAMSGGYTGGFKTIFGAGTRLFVKANIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWSS

SEQ ID NO: 111 - DNA, 819 bp, synthetic construct
  misc_feature 1..819: note = TCR-9 a complete
  source 1..819: mol_type = other DNA
atgacgtccattagagccgtgttcattttcctgtggctgcaactggacctcgtgaatggagaaaacgtcgagcagcacccatccaccctgagcgtgcaggagggagactccgcagtcatcaagtgcacttactccgactcagcatcaaactatttcccgtggtataagcaggaactcggaaagcggcctcagctcatcattgacatccgctccaacgtcggagagaagaaggaccagagaattgccgtgacactcaacaagaccgccaagcatttctcccttcacatcaccgaaacccagcccgaggacagcgccgtctacttttgtgcggcctcgaggggcactgggtttcagaagctcgtgttcggcactgggacccggctgctggtgtcgccaaacatccagaatccagaccccgcggtgtaccagctgagagactcgaagtcttccgataaatccgtgtgtctctttacagacttcgatagccagactaacgtgtcccagtccaaggactccgatgtgtacatcaccgacaagactgtgctggatatgcggagcatggactttaagtccaattcagcggtcgcgtggagcaacaagtccgacttcgcctgcgctaacgctttcaacaactccattatcccggaggataccttcttcccgtcaagcgacgtcccctgcgacgtgaagctggtcgagaagtccttcgaaaccgataccaacctgaacttccaaaacctcctagtgattgtgctcagaatcctgctgctgaaagtggctggcttcaatttgctgatgaccctgcggctctggagcagc

SEQ ID NO: 112 - DNA, 930 bp, synthetic construct
  misc_feature 1..930: note = TCR-9 b complete
  source 1..930: mol_type = other DNA
atgggttgtcggttgctgtgttgcgtcgtgttctgccttcttcaagctggtcctctcgatactgccgtgagccaaacccctaagtaccttgtcacccaaatgggcaacgacaagtccatcaaatgcgaacagaacctcggacacgacaccatgtactggtacaaacaggattccaagaagttcctgaagattatgttctcatacaacaacaaggaactgattatcaacgaaactgtgccgaaccggttctcaccgaagtcgcctgacaaggctcatctcaacttgcatatcaactcgctggagctcggcgactccgccgtgtacttctgcgcgtccagccagttctgggacggagcgggcgacgaacagtacttcggcccgggcactcggctgaccgtgaccgaagatctgaacaaagtgttcccccccgaagtggccgtgttcgaaccttccaaggcggagatcgcccacacccaaaaggccactctggtctgcctggccaccggtttcttccccgatcacgtggaactgtcttggtgggtgaacggaaaagaagtgcactcgggggtgtccacggacccccagcctctgaaggaacagccggcactgaatgactcacgctactgtctgtcgtcacggctgcgcgtgtcggccaccttctggcaaaacccgcgaaaccactttcgctgccaagtgcagttttacgggctttccgagaacgacgagtggactcaggacagagcgaagcccgtgacccaaatcgtgtccgccgaggcctggggacgcgccgactgcggtatcaccagcgccagctaccaccagggcgtgctgtcagctaccatcctttacgagatcctcctgggaaaggccaccctctacgccgtcctggtgtccgcactggtgctgatggctatggtcaagcggaaggat

SEQ ID NO: 113 - DNA, 1833 bp, synthetic construct
  misc_feature 1..1833: note = TCR-9 polycistronic construct
  source 1..1833: mol_type = other DNA
atgggttgtcggttgctgtgttgcgtcgtgttctgccttcttcaagctggtcctctcgatactgccgtgagccaaacccctaagtaccttgtcacccaaatgggcaacgacaagtccatcaaatgcgaacagaacctcggacacgacaccatgtactggtacaaacaggattccaagaagttcctgaagattatgttctcatacaacaacaaggaactgattatcaacgaaactgtgccgaaccggttctcaccgaagtcgcctgacaaggctcatctcaacttgcatatcaactcgctggagctcggcgactccgccgtgtacttctgcgcgtccagccagttctgggacggagcgggcgacgaacagtacttcggcccgggcactcggctgaccgtgaccgaagatctgaacaaagtgttcccccccgaagtggccgtgttcgaaccttccaaggcggagatcgcccacacccaaaaggccactctggtctgcctggccaccggtttcttccccgatcacgtggaactgtcttggtgggtgaacggaaaagaagtgcactcgggggtgtccacggacccccagcctctgaaggaacagccggcactgaatgactcacgctactgtctgtcgtcacggctgcgcgtgtcggccaccttctggcaaaacccgcgaaaccactttcgctgccaagtgcagttttacgggctttccgagaacgacgagtggactcaggacagagcgaagcccgtgacccaaatcgtgtccgccgaggcctggggacgcgccgactgcggtatcaccagcgccagctaccaccagggcgtgctgtcagctaccatcctttacgagatcctcctgggaaaggccaccctctacgccgtcctggtgtccgcactggtgctgatggctatggtcaagcggaaggattttagggccaaacgcgggtccggagcgaccaacttctcgctgttgaagcaggccggcgatgtggaagagaaccctggaccgatgacgtccattagagccgtgttcattttcctgtggctgcaactggacctcgtgaatggagaaaacgtcgagcagcacccatccaccctgagcgtgcaggagggagactccgcagtcatcaagtgcacttactccgactcagcatcaaactatttcccgtggtataagcaggaactcggaaagcggcctcagctcatcattgacatccgctccaacgtcggagagaagaaggaccagagaattgccgtgacactcaacaagaccgccaagcatttctcccttcacatcaccgaaacccagcccgaggacagcgccgtctacttttgtgcggcctcgaggggcactgggtttcagaagctcgtgttcggcactgggacccggctgctggtgtcgccaaacatccagaatccagaccccgcggtgtaccagctgagagactcgaagtcttccgataaatccgtgtgtctctttacagacttcgatagccagactaacgtgtcccagtccaaggactccgatgtgtacatcaccgacaagactgtgctggatatgcggagcatggactttaagtccaattcagcggtcgcgtggagcaacaagtccgacttcgcctgcgctaacgctttcaacaactccattatcccggaggataccttcttcccgtcaagcgacgtcccctgcgacgtgaagctggtcgagaagtccttcgaaaccgataccaacctgaacttccaaaacctcctagtgattgtgctcagaatcctgctgctgaaagtggctggcttcaatttgctgatgaccctgcggctctggagcagctaa

SEQ ID NO: 114 - protein, 273 aa, synthetic construct
  REGION 1..273: note = TCR-9 a complete
MTSIRAVFIFLWLQLDLVNGENVEQHPSTLSVQEGDSAVIKCTYSDSASNYFPWYKQELGKRPQLIIDIRSNVGEKKDQRIAVTLNKTAKHFSLHITETQPEDSAVYFCAASRGTGFQKLVFGTGTRLLVSPNIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWSS

SEQ ID NO: 115 - protein, 310 aa, synthetic construct
  REGION 1..310: note = TCR-9 b complete
MGCRLLCCVVFCLLQAGPLDTAVSQTPKYLVTQMGNDKSIKCEQNLGHDTMYWYKQDSKKFLKIMFSYNNKELIINETVPNRFSPKSPDKAHLNLHINSLELGDSAVYFCASSQFWDGAGDEQYFGPGTRLTVTEDLNKVFPPEVAVFEPSKAEIAHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKD

SEQ ID NO: 116 - protein, 610 aa, synthetic construct
  REGION 1..610: note = TCR-9 polyprotein
MGCRLLCCVVFCLLQAGPLDTAVSQTPKYLVTQMGNDKSIKCEQNLGHDTMYWYKQDSKKFLKIMFSYNNKELIINETVPNRFSPKSPDKAHLNLHINSLELGDSAVYFCASSQFWDGAGDEQYFGPGTRLTVTEDLNKVFPPEVAVFEPSKAEIAHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDFRAKRGSGATNFSLLKQAGDVEENPGPMTSIRAVFIFLWLQLDLVNGENVEQHPSTLSVQEGDSAVIKCTYSDSASNYFPWYKQELGKRPQLIIDIRSNVGEKKDQRIAVTLNKTAKHFSLHITETQPEDSAVYFCAASRGTGFQKLVFGTGTRLLVSPNIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWSS

(19) United States
(12) Patent Application Publication
(10) Pub. No.: US 2025/0346648 A1
(43) Pub. Date: Nov. 13, 2025

(54) MAGE A4 T CELL RECEPTORS

(71) Applicants: MEDIGENE IMMUNOTHERAPIES GMBH, Planegg-Martinsried (DE); Regeneron Pharmaceuticals, Inc., Tarrytown, NY (US)

(72) Inventors: Christian Ellinger, Munich (DE); Daniel Sommermeyer, Munich (DE); Geoffrey Parsons, Jamaica Plain, MA (US); Jasdeep Mann, Lake Forest Park, WA (US)

(21) Appl. No.: 19/217,625

(22) Filed: May 23, 2025

Related U.S. Application Data
(62) Division of application No. 17/441,861, filed on Sep. 22, 2021, now Pat. No. 12,344,654.

(30) Foreign Application Priority Data
Mar. 27, 2019 (EP) 19165387.2

(51) Int. Cl. C07K 14/725; A61K 35/17; A61K 38/00; A61P 35/00; C12N 5/0783

(52) U.S. Cl. CPC C07K 14/7051; A61K 35/17; A61P 35/00; C12N 5/0636; A61K 38/00; C07K 2319/00

(57) ABSTRACT

The present invention relates to an isolated T cell receptor (TCR) specific for MAGE-A4 and a polypeptide comprising a functional portion of the TCR. Further implicated are a multivalent TCR complex, a nucleic acid encoding a TCR, a cell expressing the TCR and a pharmaceutical composition comprising the TCR. The invention also refers to the TCR for use as a medicament, in particular to the TCR for use in the treatment of cancer.

### CROSS REFERENCE TO RELATED APPLICATIONS

This application is a divisional application of U.S. patent application Ser. No. 17/441,861, filed on Sep. 22, 2021, which is a National Stage of International Application No. PCT/EP2020/058779, filed Mar. 27, 2020, which claims the benefit of European Patent Application No. 19 165 387.2, filed Mar. 27, 2019, each of which is incorporated by reference herein in its entirety.

### STATEMENT REGARDING SEQUENCE LISTING

The application contains a Sequence Listing which has been submitted electronically in. XML format and is hereby incorporated by reference in its entirety. Said.XML copy, created on May 21, 2025, is named RPY_11502_SeqList_ST26 and is 172,070 bytes in size. The sequence listing contained in this.XML file is part of the specification and is hereby incorporated by reference herein in its entirety.

### BACKGROUND

The present invention relates to isolated T cell receptors (TCRs) specific for MAGE-A4, polypeptides comprising a functional portion of a TCR, multivalent TCR complexes, nucleic acids encoding TCRs, cell expressing TCRs, and compositions and pharmaceutical compositions comprising the same. The present invention also relates to methods of using the foregoing in methods of medical treatment or for formulation and/or use as a medicament, in particular for use in the treatment of cancer.

### Description of the Related Art

T lymphocytes (or T cells) which form part of the cell-mediated immune system play a major role in the eradication of pathogens. T cells develop in the thymus and express TCR molecules on their surface that allow the recognition of peptides presented on major histocompatibility complex (MHC) molecules which are expressed on nucleated cells (known as antigen presentation). Antigens derived from pathogens, i.e., foreign antigens presented by MHC molecules will elicit a powerful T cell response whereas self-antigens usually do not lead to a T cell response due to a negative selection of self-antigen specific T cells in the thymus during the development of such T cells. The immune system can thus discriminate between nucleated cells presenting foreign-or self-antigens and specifically target and eradicate infected cells via potent cytokine release and cellular cytotoxicity mechanisms of the T cells.

The power of the immune system has been recognized as a promising tool for future cancer therapies. In the last decades, research has begun to exploit the unique properties of T cells by using adoptive cell therapy (ACT), which involves the administration of patient-derived lymphocytes, expanded ex vivo. ACT is an attractive concept for the treatment of cancer because it does not require immune-competence of patients, and the specificity of transferred lymphocytes can be targeted against non-mutated and thus poorly immunogenic tumor antigens that typically fail to effectively trigger autologous T cell responses. Although ACT has been shown to be a promising treatment for various types of cancer, its broad application as clinical treatment has been hampered by the need for custom isolation and characterization of tumor-specific T cells from each patient-a process that can be not only difficult and time-consuming but also often fails to yield high-avidity T cells (Xue et al. Clin. Exp. Immunol. 2005 February; 139(2): 167-172; Schmitt et al., Hum. Gene Ther. 2009 November; 20(11): 1240-1248.)

The genetic transfer of tumor antigen-specific TCRs into primary T cells can overcome some of the current limitations of ACT, as it allows for the rapid generation of tumor-reactive T lymphocytes with defined antigen specificity even in immunocompromised patients. However, the identification of suitable T cell clones bearing TCRs that specifically recognize tumor antigens and exhibit the desired anti-tumor effects in vivo is still the topic of ongoing research. Considering that in 2012 about 14.1 million new cases of cancer occurred globally and that cancer currently is the cause of about 14.6% of all human deaths worldwide, novel and efficient treatment options are urgently needed. It is an object of the present invention to comply with the needs set out above.

MAGE-A4 belongs to the melanoma antigen (MAGE) family. The MAGE family is expressed in various malignant tumor types, ranging from melanoma, colon, lung to breast and other tumors. Specifically, the MAGE family is divided into two groups, based on its tissue expression pattern, where the MAGE-A subfamily is expressed in germ cells of the testis and aberrantly re-expressed in malignant tumors. This also applies to MAGE-A4.

Tumor tissue expression studies have revealed that MAGE-A4 is (over) expressed in 19-35% of non-small cell lung cancer (NSCLC) cases, 13% of breast cancer cases, 47% in epithelial ovarian cancer cases and 22% in colorectal cancer cases (Tajima et al. Lung Cancer 2003; 42:23-33; Gure et al. Clin Cancer Res 2005, 11:8055-8062; Kim et al. Int J Mol Med 2012, 29:656-662; Otte et al. Cancer Res 2001, 61:6682-668; Daudi et al. PLOS One 2014, 9: e104099; Li et al. Clin Cancer Res 2005, 11:1809-1814).

### BRIEF SUMMARY

It is an objective of the invention to provide an isolated T cell receptor (TCR) specific for MAGE-A4.

In particular, the TCR specifically recognizes the amino acid sequence SEQ ID NO: 1 or a fragment thereof.

In specific embodiments, the TCR specifically recognizes the HLA-A2 bound form of the amino acid sequence of SEQ ID NO: 1, more specifically the TCR recognizes the amino acid sequence of SEQ ID NO: 1, which is presented by the HLA-A*02:01 encoded molecule.

In some embodiments, the TCR comprises: a variable TCR α region comprising a CDR1 having the amino acid sequence of SEQ ID NO: 2, a CDR2 having the amino acid sequence of SEQ ID NO: 3 and a CDR3 having the amino acid sequence of SEQ ID NO: 4, and a variable TCR β region comprising a CDR1 having the amino acid sequence of SEQ ID NO: 5, a CDR2 having the amino acid sequence of SEQ ID NO: 6 and a CDR3 having the amino acid sequence of SEQ ID NO: 7; or a variable TCR α region comprising a CDR1 having the amino acid sequence of SEQ ID NO: 12, a CDR2 having the amino acid sequence of SEQ ID NO: 13 and a CDR3 having the amino acid sequence of SEQ ID NO: 14, and a variable TCR β region comprising a CDR1 having the amino acid sequence of SEQ ID NO: 15, a CDR2 having the amino acid sequence of SEQ ID NO: 16 and a CDR3 having the amino acid sequence of SEQ ID NO: 17; or a variable TCR α region comprising a CDR1 having the amino acid sequence of SEQ ID NO: 22, a CDR2 having the amino acid sequence of SEQ ID NO: 23 and a CDR3 having the amino acid sequence of SEQ ID NO: 24, and a variable TCR β region comprising a CDR1 having the amino acid sequence of SEQ ID NO: 25, a CDR 2 having the amino acid sequence of SEQ ID NO: 26 and a CDR 3 having the amino acid sequence of SEQ ID NO: 27. These TCRs are described in more detail below.

In particular embodiments a TCR comprises: a variable TCR α region having the amino acid sequence of SEQ ID NO: 8 and a variable TCR β region having the amino acid sequence of SEQ ID NO: 9; or a variable TCR α region having the amino acid sequence of SEQ ID NO: 18 and a variable TCR β region having the amino acid sequence of SEQ ID NO: 19; or a variable TCR α region having the amino acid sequence of SEQ ID NO: 28 and a variable TCR β region having the amino acid sequence of SEQ ID NO: 29.

In certain embodiments, a TCR comprises: a TCR α chain having the amino acid sequence of SEQ ID NO: 10 and a TCR β chain having the amino acid sequence of SEQ ID NO: 11; a TCR α chain having the amino acid sequence of SEQ ID NO: 20 and a TCR β chain having the amino acid sequence of SEQ ID NO: 21; or a TCR α chain having the amino acid sequence of SEQ ID NO: 30 and a TCR β chain having the amino acid sequence of SEQ ID NO: 31.

In other embodiments, a TCR comprises: a TCR α chain having the amino acid sequence of SEQ ID NO: 87 and a TCR β chain having the amino acid sequence of SEQ ID NO: 88; a TCR α chain having the amino acid sequence of SEQ ID NO: 89 and a TCR β chain having the amino acid sequence of SEQ ID NO: 90; or a TCR α chain having the amino acid sequence of SEQ ID NO: 91 and a TCR β chain having the amino acid sequence of SEQ ID NO: 92.

In some embodiments, the TCR comprises: a TCR α chain having the amino acid sequence of SEQ ID NO: 102 and a TCR β chain having the amino acid sequence of SEQ ID NO: 103; a TCR α chain having the amino acid sequence of SEQ ID NO: 108 and a TCR β chain having the amino acid sequence of SEQ ID NO: 109; or a TCR α chain having the amino acid sequence of SEQ ID NO: 114 and a TCR β chain having the amino acid sequence of SEQ ID NO: 115.

TCRs contemplated herein are isolated and/or purified and may be soluble or membrane bound.

In some embodiments, the invention refers to an isolated TCR described herein, wherein the IFN-γ secretion induced by binding of the TCR expressed on an effector cell to the amino acid sequence of SEQ ID NO: 1, which is presented by the HLA-A*02:01 encoded molecule, may be more than 100 times higher, preferably 500 times higher, more preferably 2000 times higher when binding to the amino acid sequence of SEQ ID NO: 1, which is presented by the HLA-A*02:01 encoded molecule, compared to binding to an irrelevant peptide, which is presented by the HLA-A*02:01 encoded molecule.

In some embodiments, the amino acid sequence of the TCR may comprise one or more phenotypically silent substitutions. In addition, the TCRs can be labelled. Useful labels are known in the art and can be coupled to the TCR or TCR variant using routine methods, optionally via linkers of various lengths. The term “label” or “labelling group” refers to any detectable label. Additionally, or alternatively, the amino acid sequence may be modified to comprise a therapeutic agent or pharmacokinetic modifying moiety. The therapeutic agent may be selected from the group consisting of an immune effector molecule, a cytotoxic agent and a radionuclide. The immune effector molecule may for example be a cytokine. The pharmacokinetic modifying moiety may be at least one polyethylene glycol repeating unit, at least one glycol group, at least one sialyl group or a combination thereof.

The TCR, in particular a soluble form of the TCR contemplated herein can be modified by attaching additional functional moieties, e.g., for reducing immunogenicity, increasing hydrodynamic size (size in solution) solubility and/or stability (e.g., by enhanced protection to proteolytic degradation) and/or extending serum half-life. Other useful functional moieties and modifications include “suicide” or “safety switches” that can be used to shut off or turn on effector host cells carrying an inventive TCR in a patient's body. TCRs with an altered glycosylation pattern are also envisaged herein.

It is also conceivable to add a drug or a therapeutic entity, such as a small molecule compound to the TCR, in particular to a soluble form of the inventive TCR. The TCR, in particular a soluble form of the inventive TCR can additionally be modified to introduce additional domains which aid in identification, tracking, purification and/or isolation of the respective molecule (tags).

In some embodiments, a TCR is of the single chain type, wherein the TCR α chain and the TCR β chain are linked by a linker sequence, optionally wherein the linker sequence is cleavable.

Another aspect refers to a polypeptide comprising a functional portion of the TCR as described herein, wherein the functional portion comprises at least one of the amino acid sequences selected from the group consisting of SEQ ID NOs: 4, 7, 14, 17, 24 and 27.

In specific embodiments, the functional portion comprises the TCR α variable region and/or the TCR β variable region.

Specific embodiments refer to a multivalent TCR complex comprising at least two TCRs as described herein. In a more specific embodiment, at least one of said TCRs is associated with a therapeutic agent.

Particular embodiments refer to a fusion protein comprising a TCR α chain and a TCR β chain, wherein the fusion protein comprises the amino acid sequence set forth in any one of SEQ ID NOs: 94, 96, 98, 104, 110, and 116.

In specific aspects the fusion protein further comprises a furin cleavage site and/or a ribosomal skip sequence.

Another aspect refers to a nucleic acid encoding a TCR as described herein or encoding the polypeptide or fusion protein as described above.

In one aspect, a nucleic acid sequence encoding the TCRα chain is set forth in any one of SEQ ID NOs: 69, 77, 85, 99, 105, and 111. In another aspect, the nucleic acid sequence encoding the TCRβ chain is set forth in any one of SEQ ID NOs: 70, 78, 86, 100, 106, and 112. In other aspects, a TCR comprises an a chain encoded by SEQ ID NO: 69 and a β chain encoded by SEQ ID NO: 70;an α chain encoded by SEQ ID NO: 77 and a β chain encoded by SEQ ID NO: 78; an α chain encoded by SEQ ID NO: 85 and a β chain encoded by SEQ ID NO: 86; an α chain encoded by SEQ ID NO: 99 and a β chain encoded by SEQ ID NO: 100; an α chain encoded by SEQ ID NO: 105 and a β chain encoded by SEQ ID NO: 106; or an α chain encoded by SEQ ID NO: 111 and a β chain encoded by SEQ ID NO: 112.

Further aspects relate to a fusion protein encoded by the nucleic acid sequence set forth in any one of SEQ ID NOs: 93, 95, 97, 101, 107, and 113.

A further aspect refers to a plasmid or vector comprising the nucleic acid of the present application as described above. A further aspect refers to a plasmid or vector comprising a nucleic acid encoding the polypeptide sequences set forth in SEQ ID NO: 87 and SEQ ID NO: 88; the polypeptide sequences set forth in SEQ ID NO: 89 and SEQ ID NO: 90; the polypeptide sequences set forth in SEQ ID NO: 91 and SEQ ID NO: 92; the polypeptide sequences set forth in SEQ ID NO: 102 and SEQ ID NO: 103; the polypeptide sequences set forth in SEQ ID NO: 108 and SEQ ID NO: 109; or the polypeptide sequences set forth in SEQ ID NO: 114 and SEQ ID NO: 115. Preferably, the vector is an expression vector or a vector suitable for the transduction or transfection of cells, especially eukaryotic cells. The vector may be for example a retroviral vector, for example a gamma-retroviral or lentiviral vector.

Another aspect refers to a cell expressing a TCR as described herein. The cell may be isolated or non-naturally occurring.

Another aspect refers to a cell comprising the nucleic acid as described above or the plasmid or vector as described above. More specifically, the cell may comprise: an expression vector which comprises a nucleic acid or multiple nucleic acids as described above; or a first expression vector which comprises a nucleic acid encoding the alpha chain of the TCR as described herein, and a second expression vector which comprises a nucleic acid encoding the beta chain of a TCR as described herein.

The cell may be a peripheral blood lymphocyte (PBL) or a peripheral blood mononuclear cell (PBMC). Typically, the cell is an immune effector cell, especially a T cell. Other suitable cell types include gamma-delta T cells, natural killer (NK) cells, and NK-like T (NKT) cells.

Another aspect refers to an antibody or antigen binding fragment thereof specifically binding to a portion of the TCR as described herein which mediates specificity for MAGE-A4.

Another aspect refers to a composition comprising the TCR as described herein, the polypeptide as described herein, the fusion protein described herein, the multivalent TCR complex as described herein, the nucleic acid as described herein, the vector as described herein, the cell as described herein, or the antibody as described herein.

Another aspect refers to a pharmaceutical composition comprising the TCR as described herein, the polypeptide as described herein, the fusion protein described herein, the multivalent TCR complex as described herein, the nucleic acid as described herein, the vector as described herein, the cell as described herein, or the antibody as described herein.

Typically, the pharmaceutical composition comprises at least one pharmaceutically acceptable carrier.

Another aspect refers to TCR as described herein, the polypeptide as described herein, the multivalent TCR complex as described herein, the nucleic acid as described herein, the vector as described herein, the cell as described herein, the antibody as described herein, the composition described herein, or the pharmaceutical composition for use as a medicament, in particular for use in the treatment of cancer. The cancer may be a hematological cancer or a solid tumor. The cancer may be selected from the group consisting sarcoma, prostate cancer, uterine cancer, thyroid cancer, testicular cancer, renal cancer, pancreatic cancer, ovarian cancer, esophageal cancer, non-small-cell lung cancer, non-Hodgkin's lymphoma, multiple myeloma, melanoma, hepatocellular carcinoma, head and neck cancer, gastric cancer, endometrial cancer, colorectal cancer, cholangiocarcinoma, breast cancer, bladder cancer, myeloid leukemia and acute lymphoblastic leukemia. Preferably, the cancer is selected from the group consisting of NSCLC, SCLC, breast, ovarian or colorectal cancer, sarcoma and osteosarcoma.

### BRIEF DESCRIPTION OF SEVERAL VIEWS OF THE DRAWINGS

FIG. 1 shows the MAGE-A4_{GVY}-MHC-multimer binding of CD8+ T cells transduced with different MAGE-A4-reactive TCRs. CD8+ T cells were isolated from PBMCs of a healthy donor and transduced with three different MAGE-A4-TCRs and one control TCR that did not recognize MAGE-A4. Transduced CD8+ T cells were enriched by FACS using the murine constant beta region as a marker for transduction. After expansion of these cells, they were stained with an MAGE-A4_{GVY}-MHC-multimer and antibodies against CD8 and the murine constant beta region (mmCb) and analyzed by flow cytometry. Populations were gated on live CD8+/mCb+ cells and staining of multimer/CD8 is shown.

FIG. 2 shows that MAGE-A4-TCR-transgenic T cells recognize MAGE-A4_{GVY}-peptide presented on HLA-A2. Transgenic T cells were co-cultured with T2 cells externally loaded with MAGE-A4_{GVY}-peptide or K562/HLA-A2 cells that had been transduced with the MAGE-A4 gene. As negative controls, T2 cells loaded with a control peptide and untransduced K562/HLA-A2 cells were used, respectively. Recognition of target cells was analyzed by measuring the IFN-γ concentration in co-culture supernatants by a standard ELISA.

FIG. 3 shows the functional avidity of MAGE-A4-TCR-transgenic T cells. Transgenic T cells were co-cultured with T2 cells externally loaded with graded concentrations of MAGE-A4_{GVY}-peptide (10-12 M-10-4 M). IFN-γ concentration in co-culture supernatants was measured by a standard ELISA.

FIGS. 4A-C show the ability of MAGE-A4-TCR-transgenic T cells (TCR-1, TCR-2 and TCR-3) to lyse MAGE-A4-positive tumor cell lines in a HLA-A2-dependent manner. Transgenic T cells were co-cultured with different MAGE-A4-positive HLA-A2-positive tumor cell lines (NCI-H1703, NCI-H1755), a MAGE-A4-negative HLA-A2-positive tumor cell line (Saos-2) and a MAGE-A4-negative HLA-A2-negative tumor cell line (A549). Tumor cells loaded with MAGE-A4_{GVY}-peptide are used as positive control. Cytotoxicity against the tumor cell lines stably transduced with a fluorescence marker was measured with an IncuCyte® ZOOM device (Essen Bioscience) by taking pictures every two hours. To analyze cytokine release, co-culture supernatants were harvested after 24 hrs. and IFN-γ concentrations analyzed by standard sandwich ELISA (BD human IFN-γ ELISA set).

FIGS. 5A-C show that MAGE-A4-TCR-transgenic T cells (TCR-1, TCR-2 and TCR-3) do not recognize normal human cells. Transgenic T cells were co-cultured with different primary cells and induced pluripotent stem cell (iPS)-derived cells representing essential tissues or organs. Normal cells loaded with MAGE-A4_{GVY}-peptide are used as positive control. HLA-A2 expression was induced on neurons by pre-incubation with IFN-γ. The HLA-A2-negative NHBE cells were electroporated with HLA-A2-ivtRNA and HLA-A2 expression of all cells was confirmed via flow cytometry. To analyze cytokine release, co-culture supernatants were harvested after 24 h and IFN-γas well as IL-2 concentrations were analyzed by standard sandwich ELISA (BD human IFN-γor IL-2 ELISA set).

FIG. 6 shows the MAGE-A4_{GVY}-MHC-multimer binding of CD3+ T cells transduced with different MAGE-A4-reactive fully human TCRs. CD3+ T cells were isolated from PBMCs of a healthy donor and transduced with three different MAGE-A4-TCRs and one control TCR that did not recognize MAGE-A4. After expansion of these cells, they were stained with a MAGE-A4_{GVY}-MHC-multimer and antibody against CD3 and analyzed by flow cytometry. Populations were gated on live CD3+ cells and multimer staining.

FIG. 7 shows that MAGE-A4 fully human TCR-transgenic T cells recognize MAGE-A4_{GVY}-peptide presented on HLA-A2. Transgenic T cells were co-cultured with T2 cells externally loaded with MAGE-A4_{GVY}-peptide or A549/HLA-A2 cells that had been transduced with the MAGE-A4 gene. As negative controls, T2 cells loaded with a control peptide and untransduced A549/HLA-A2 cells were used, respectively. Recognition of target cells was analyzed by measuring the IFN-γ concentration in co-culture supernatants by a Luminex assay.

FIG. 8 shows the ability of MAGE-A4 fully human TCR-transgenic T cells to specifically react to MAGE-A4-positive tumor cell lines in an HLA-A2-dependent manner. Transgenic T cells were co-cultured with different MAGE-A4-positive HLA-A2-positive tumor cell lines (A375, NCI-H1703, NCI-H1755), a MAGE-A4-positive HLA-A2-negative tumor cell line (NCI-H520), and a MAGE-A4-negative HLA-A2-positive tumor cell line (A549). To analyze cytokine release, co-culture supernatants were harvested after 24 hrs. and IFN-γ concentrations analyzed by Luminex assay.

FIG. 9 shows the ability of MAGE-A4 fully human TCR-transgenic T cells to lyse MAGE-A4-positive tumor cell lines in a HLA-A2-dependent manner. Transgenic T cells were co-cultured with different MAGE-A4-positive HLA-A2-positive tumor cell lines (A375, NCI-H1755, A549-HLA-A2-MAGE-A4) and a MAGE-A4-negative HLA-A2-positive tumor cell line (A549-HLA-A2). Cytotoxicity against the tumor cell lines was measured by an impedance assay beginning 6 hours after co-culture initiation.

FIG. 10 shows the ability of a MAGE-A4 fully human TCR-transgenic T cells to control MAGE-A4-positive tumors engrafted in NSG mice.

FIGS. 11A-11C show the vector copy number (VCN) and expression of MAGE-A4 TCRs. Peripheral blood mononuclear cells (PBMCs) were transduced with lentiviral vectors encoding a fully human MAGE-A4 TCR (TCR-5) or enhanced variant (TCR-8). 11A) VCN measurements in TCR-5 and TCR-8 T cells were comparable. 11B) TCR expression on the T cell surface was evaluated using GVY-specific tetramer detection by flow cytometry and shown as percentage of total CD3+ T cells. TCR-8 expression is increased compared to TCR-5 expression. 11C) The density of TCR molecules on the T cell surface was evaluated using GVY-specific tetramer detection by flow cytometry and shown as geometric Mean Fluorescence Intensity (gMFI) of total Tetramer+TCR T cells. Expression density of TCR-8 is increased compared to TCR-5 expression density.

FIGS. 12A-12B show that T cells expressing a fully human MAGE-A4 TCR (TCR-5) or enhanced variant (TCR-8) specifically kill MAGE-A4 expressing target cells in vitro. 12A) TCR-5, TCR-8, or untransduced (UTD) T cells were co-cultured with A549.A2 cells (A2+, MAGE-A4(−)), NCI-H2023 cells (A2+, MAGE-A4(+)), A375 cells (A2+, MAGE-A4(+)), or A549.A2.MAGEA4 cells (A2+, MAGE-A4(+)) at 1:1 E:T ratio. IFNγ release was evaluated as a biomarker for T-cell activity after 24hrs. TCR-5 and TCR-8 T cells secreted INFγ when co-cultured targets cells expressing MAGE-A4 but not in the presence of MAGE-A4 negative. 12B) TCR-5, TCR-8, or untransduced (UTD) T cells were co-cultured with A375 cells (A2+, MAGE-A4(+)), A549.A2.MAGEA4 cells (A2+, MAGE-A4(+)), or U2OS cells (A2+, MAGE-A4(low)), at 10:1, 5:1 and 2.5:1 E:T ratios. Cytotoxicity was measured as normalized percentage over tumor cell alone after 6 hours by means of impedance. TCR-5 and TCR-8 T cells mediated comparable cytotoxicity against the three MAGE-A4 expressing cell lines.

FIGS. 13A-13C show that T cells expressing a fully human MAGE-A4 TCR (TCR-5) or enhanced variant (TCR-8) mediate regression in mice with MAGE-A4 expressing tumors. 5 NSG mice (each condition) were injected subcutaneously with MAGEA4(+) A375 tumor cells, and treated with Vehicle, UTD T cells, or T cells expressing a fully human MAGE-A4 TCR (TCR-5) or enhanced variant (TCR-8). Tumor growth was measured twice a week and TCR T cells anti-tumor activity was evaluated in comparison to mice receiving UTD and Vehicle controls. A and B) mice with 50 mm³ A375 tumors received 5×10⁶ (left flank, A) or 1.5×10⁶ (right flank, B) UTD T cells, TCR-5 T cells, or TCR-8 T cells. Both TCR-5 and TCR-8 T cells controlled tumors at a dose of 5×10⁶ T cells, but TCR-8 T cells showed increased control of tumors at the lower dose of 1.5×10⁶ T cells. C) mice with 100 mm³ A375 tumors received 10×10⁶ UTD T cells, TCR-5 T cells, or TCR-8 T cells. TCR-8 T cells mediated increased tumor regression compared to TCR-5 T cells or UTD T cells.

### DETAILED DESCRIPTION

### A. Overview

The MAGE-A4 expression pattern makes it a suitable tumor specific target for ACT. MAGE-A4 comprises an epitope in form of a decapeptide (Duffour et al. Eur J Immunol. 1999 10:3329-37) having the amino acid sequence GVYDGREHTV (SEQ ID NO: 1) which is presented by HLA-A2 molecules. Taken together, MAGE-A4 is a suitable tumor specific target for ACT. Novel, safe and effective TCR effectors targeting MAGE-A antigens for cancer immunotherapy are needed. The present disclosure relates to T cell receptors that efficiently target MAGE-A4 antigens on cancer cells and aims to address this unmet medical need.

### B. Definitions

Before aspects of the invention are described in more detail with respect to some of its preferred embodiments, the following general definitions are provided.

The present invention as illustratively described in the following may suitably be practiced in the absence of any element or elements, limitation or limitations, not specifically disclosed herein.

The present invention will be described with respect to particular embodiments and with reference to certain figures but the invention is not limited thereto but only by the claims.

Where the term “comprising” is used in the present description and claims, it does not exclude other elements. For the purposes of the present invention, the term “consisting of” is considered to be a preferred embodiment of the term “comprising of”. If hereinafter a group is defined to comprise at least a certain number of embodiments, this is also to be understood to disclose a group, which preferably consists only of these embodiments.

For the purposes of the present invention, the term “obtained” is considered to be a preferred embodiment of the term “obtainable”. If hereinafter e.g., an antibody is defined to be obtainable from a specific source, this is also to be understood to disclose an antibody, which is obtained from this source.

Where an indefinite or definite article is used when referring to a singular noun, e.g. “a,” “an” or “the”, this includes a plural of that noun unless something else is specifically stated. The terms “about” or “approximately” in the context of the present invention denote an interval of accuracy that the person skilled in the art will understand to still ensure the technical effect of the feature in question. The term typically indicates deviation from the indicated numerical value of ±10%, and preferably of ±5%, more preferably of ±2%, most preferably of ±1%.

Unless otherwise indicated, the term “at least” preceding a series of elements is to be understood to refer to every element in the series. The term “at least one” refers to one or more such as two, three, four, five, six, seven, eight, nine, ten or more. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and,” “or,” and “all or any other combination of the elements connected by said term”.

The term “less than” or in turn “more than” does not include the concrete number. For example, less than 20 means less than the number indicated. Similarly, more than or greater than means more than or greater than the indicated number, e.g., more than 80% means more than or greater than the indicated number of 80%.

The term “including” means “including but not limited to.” “Including” and “including but not limited to” are used interchangeably.

Throughout the description and claims of this specification, the singular encompasses the plural unless the context otherwise requires. In particular, where the indefinite article is used, the specification is to be understood as contemplating plurality as well as singularity, unless the context requires otherwise.

Technical terms are used by their common sense or meaning to the person skilled in the art. If a specific meaning is conveyed to certain terms, definitions of terms will be given in the following in the context of which the terms are used.

Additional definitions are set forth throughout this disclosure.

All publications cited throughout the text of this specification (including all patents, patent application, scientific publications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

The content of all documents and patent documents cited herein is incorporated by reference in their entirety.

### C. TCR Background

A TCR is composed of two different and separate protein chains, namely the TCR alpha (a) and the TCR beta (B) chain. The TCR α chain comprises variable (V), joining (J) and constant (C) regions. The TCR β chain comprises variable (V), diversity (D), joining (J) and constant (C) regions. The rearranged V(D)J regions of both the TCR α and the TCR β chain contain hypervariable regions (CDR, complementarity determining regions), among which the CDR3 region determines the specific epitope recognition. At the C-terminal region both the TCR α chain and TCR β chain contain a hydrophobic transmembrane domain and end in a short cytoplasmic tail.

Typically, the TCR is a heterodimer of one α chain and one β chain. This heterodimer can bind to MHC molecules presenting a peptide.

The term “variable TCR α region” or “TCR α variable chain” or “variable domain” in the context refers to the variable region of a TCR α chain. The term “variable TCR β region” or “TCR β variable chain” in the context refers to the variable region of a TCR β chain.

The TCR loci and genes are named using the International Immunogenetics (IMGT) TCR nomenclature (IMGT Database, www. IMGT.org; Giudicelli et al. Nucl. Acids Res., 34, D781-D784(2006).; Lefranc and Lefranc, Academic Press 2001).

### D. MAGE-A4

A first aspect relates to an isolated T cell receptor (TCR) specific for MAGE-A4.

MAGE-A4 belongs to the group of so-called Cancer/Testis antigens. Cancer/Testis antigens are expressed in various malignant tumors and germ cells but in no other adult tissues. Therefore, MAGE-A4 is an interesting immunotherapeutic target antigen. The human gene encoding MAGE-A4 is designated MAGEA4 (ENSG00000147381).

In particular, a TCR contemplated herein specifically recognizes the epitope comprising the amino acids 230 to 239 of MAGE-A4, i.e., the amino acid sequence SEQ ID NO: 1 (GVYDGREHTV; also denoted herein as MAGE-A4_{GVY}) or a fragment thereof.

Typically, the TCR recognizes the peptide fragment of the antigen when it is presented by a major histocompatibility complex (MHC) molecule.

The human leukocyte antigen (HLA) system or complex is a gene complex encoding the major histocompatibility complex (MHC) proteins in humans. HLA-A*02 (HLA-A2) is one particular class I major histocompatibility complex (MHC) allele group at the HLA-A locus. HLA-A*02:01 is a specific HLA-A*02 allele.

Thus, in a specific embodiment, the TCR specifically recognizes the HLA-A2 bound form of the amino acid sequence of SEQ ID NO: 1. In an even more specific embodiment, the TCR specifically recognizes amino acid sequence of SEQ ID NO: 1, which is presented by the HLA-A*02:01 encoded molecule.

The TCR is highly specific for MAGE-A4 and exhibits no cross-reactivity to other peptides. That means that the TCR does not recognize normal human cell lines including cardiomyocytes, endothelial cells, lung fibroblasts, hepatocytes, renal cortical epithelial cells, astrocytes, bronchial epithelial cells and neurons that do not express MAGE-A4. The cross-reactivity may be measured by IFN-γ secretion as described herein.

The term “specific for” in the context means that the TCR is specifically binding to the target. In particular embodiments, a TCR is specific for MAGE-A4, and specifically binds to the amino acid sequence set forth in SEQ ID NO: 1 presented by an HLA-A*02:01 encoded molecule.

### E. TCR Specific Sequence

The CDR3 of the TCR α chain of the TCR may have the amino acid sequence selected from the group consisting of SEQ ID NO: 4, SEQ ID NO: 14 and SEQ ID NO: 24.

The CDR3 of the TCR β chain of the TCR may have the amino acid sequence selected from the group consisting of SEQ ID NO: 7, SEQ ID NO: 17 and SEQ ID NO: 27.

Some embodiments relate to an isolated TCR comprising a TCR α chain and a TCR β chain, wherein a) the TCR α chain comprises a complementarity-determining region 3 (CDR3) having the amino acid sequence of SEQ ID NO: 4, and the TCR β chain comprises a CDR3 having the amino acid sequence of SEQ ID NO: 7; or b) the TCR α chain comprises a CDR3 having the amino acid sequence of SEQ ID NO: 14, and the TCR β chain comprises a CDR3 having the amino acid sequence of SEQ ID NO: 17; or c) the TCR α chain comprises a CDR3 having the amino acid sequence of SEQ ID NO: 24, and the TCR β chain comprises a CDR3 having the amino acid sequence of SEQ ID NO: 27.

More specific embodiments relate to an isolated TCR, wherein the TCR comprises: a) a TCR α chain comprising a CDR1 having the amino acid sequence of SEQ ID NO: 2, a CDR 2 having the amino acid sequence of SEQ ID NO: 3 and a CDR 3 having the amino acid sequence of SEQ ID NO: 4, and a TCR β chain comprising a CDR1 having the amino acid sequence of SEQ ID NO: 5, a CDR 2 having the amino acid sequence of SEQ ID NO: 6 and a CDR 3 having the amino acid sequence of SEQ ID NO: 7; or b) a TCR α chain comprising a CDR1 having the amino acid sequence of SEQ ID NO: 12, a CDR 2 having the amino acid sequence of SEQ ID NO: 13 and a CDR 3 having the amino acid sequence of SEQ ID NO: 14, and a TCR β chain comprising a CDR1 having the amino acid sequence of SEQ ID NO: 15, a CDR 2 having the amino acid sequence of SEQ ID NO: 16 and a CDR 3 having the amino acid sequence of SEQ ID NO: 17; or c) a TCR α chain comprising a CDR1 having the amino acid sequence of SEQ ID NO: 22, a CDR2 having the amino acid sequence of SEQ ID NO: 23 and a CDR3 having the amino acid sequence of SEQ ID NO: 24, a TCR β chain comprising a CDR1 having the amino acid sequence of SEQ ID NO: 25, a CDR 2 having the amino acid sequence of SEQ ID NO: 26 and a CDR 3 having the amino acid sequence of SEQ ID NO: 27.

Preferred embodiments relate to isolated TCRs which are defined by the CDRs, in particular by the CDR3 of the TCR α and the TCR β chain as described above, wherein the recombinant TCR sequence is modified to contain murinized, preferably minimal murinized Cα and Cβ regions.

In particularly preferred embodiments, isolated TCRs are defined by the CDRs, in particular by the CDR3 of the TCR α and the TCR β chain as described above, wherein the recombinant TCR sequence is modified to contain minimal murinized Cα and Cβ regions and hydrophobic amino acid mutations in the Cα transmembrane domain. In particular embodiments, these TCRs have increased expression and functional avidity compared to TCRs that are not minimally murinized and do not contain hydrophobic mutations in the Cα transmembrane region.

In further preferred embodiments, isolated TCRs are defined by the CDRs, in particular by the CDR3 of the TCR α and the TCR β chain as described above, wherein the recombinant TCR sequence is not modified to contain murinized, or minimally murinized Cα and Cβ regions.

Some embodiments refer to an isolated TCR, wherein the TCR comprises: a) a variable TCR a region having an amino acid sequence which is at least 80% identical to SEQ ID NO: 8 and a variable TCR β region having an amino acid sequence which is at least 80% identical to SEQ ID NO: 9; or b) a variable TCR α region having an amino acid sequence which is at least 80% identical to SEQ ID NO: 18 and a variable TCR β region having an amino acid sequence which is at least 80% identical to SEQ ID NO: 19; or c) a variable TCR α region having an amino acid sequence which is at least 80% identical to SEQ ID NO: 28 and a variable TCR β region having an amino acid sequence which is at least 80% identical to SEQ ID NO: 29.

“At least 80% identical”, in particular “having an amino acid sequence which is at least 80% identical” as used herein includes that the amino acid sequence is at least 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence set out.

In some embodiments the TCR comprises a TCR α chain and a TCR β chain, wherein a) the variable TCR α region has an amino acid sequence which is at least 80% identical to SEQ ID NO: 8 and comprises a CDR3 having the amino acid sequence set out in SEQ ID NO: 4, and the variable TCR β region has an amino acid sequence which is at least 80% identical to SEQ ID NO: 9 and comprises a CDR3 having the amino acid sequence set out SEQ ID NO: 7 or b) the variable TCR α region has an amino acid sequence which is at least 80% identical to SEQ ID NO: 18 and comprises a CDR3 having the amino acid sequence set out in SEQ ID NO: 14, and the variable TCR β region has an amino acid sequence which is at least 80% identical to SEQ ID NO: 19 and comprises a CDR3 having the amino acid sequence set out SEQ ID NO: 17; or c) the variable TCR α region has an amino acid sequence which is at least 80% identical to SEQ ID NO: 28 and comprises a CDR3 having the amino acid sequence set out in SEQ ID NO: 24; and the variable TCR β region has an amino acid sequence which is at least 80% identical to SEQ ID NO: 29 and comprises a CDR3 having the amino acid sequence set out SEQ ID NO: 27.

Exemplary embodiments refer to an isolated TCR, wherein the TCR comprises: a) a variable TCR α region having the amino acid sequence of SEQ ID NO: 8 and a variable TCR β region having the amino acid sequence of SEQ ID NO: 9; or b) a variable TCR α region having the amino acid sequence of SEQ ID NO: 18 and a variable TCR β region having the amino acid sequence of SEQ ID NO: 19; or c) a variable TCR α region having the amino acid sequence of SEQ ID NO: 28 and a variable TCR β region having the amino acid sequence of SEQ ID NO: 29.

The following table shows a summary of the exemplary TCRs

| TCR | CDR1α | CDR2α | CDR3α | TRAV | TRAJ | CDR1β | CDR2β | CDR3β | TRBV | TRBJ |
|---|---|---|---|---|---|---|---|---|---|---|
| 1, 4, 7 | TSDQSYG | QGSYDEQN | CAMSGDSAGNMLTF | 14 | 39 | KGHDR | SFDVKD | CATSDWDRSGDKETQYF | 24 | 2-5 |
| 2, 5, 8 | TSDPSYG | QGSYDQQN | CAMSGGYTGGFKTIF | 14 | 9 | SGDLS | YYNGEE | CASSGGDGDEQFF | 9 | 2-1 |
| 3, 6, 9 | DSASNY | IRSNVGE | CAASRGTGFQKLVF | 13 | 8 | LGHDT | YNNKEL | CASSQFWDGAGDEQYF | 3-1 | 2-7 |

As can be seen from the examples, the TCRs contemplated herein are specific for MAGE-A4.

The determination of percent identity between multiple sequences is preferably accomplished using the AlignX application of the Vector NTI Advance™ 10 program (Invitrogen Corporation, Carlsbad CA, USA). This program uses a modified Clustal W algorithm (Thompson et al. Nucl Acids Res 1994; 42:23-33; Invitrogen Corporation. User Manual 2004; 389-662). The determination of percent identity is performed with the standard parameters of the AlignX application.

The TCR contemplated herein are isolated or purified. “Isolated” means that the TCR is not present in the context in which it originally occurred in nature. “Purified” means e.g., that the TCR is free or substantially free of other proteins and non-protein parts of the cell it originally stems from.

In some embodiments, the amino acid sequence of the TCR may comprise one or more phenotypically silent substitutions.

“Phenotypically silent substitutions” are also named “conservative amino acid substitutions.” The concept of “conservative amino acid substitutions” is understood by the skilled artisan, and preferably means that codons encoding positively-charged residues (H, K, and R) are substituted with codons encoding positively-charged residues, codons encoding negatively-charged residues (D and E) are substituted with codons encoding negatively-charged residues, codons encoding neutral polar residues (C, G, N, Q, S, T, and Y) are substituted with codons encoding neutral polar residues, and codons encoding neutral non-polar residues (A, F, I, L, M, P, V, and W) are substituted with codons encoding neutral non-polar residues. These variations can spontaneously occur, be introduced by random mutagenesis, or can be introduced by directed mutagenesis. Those changes can be made without destroying the essential characteristics of these polypeptides. The ordinarily skilled artisan can readily and routinely screen variant amino acids and/or the nucleic acids encoding them to determine if these variations substantially reduce or destroy the ligand binding capacity by methods known in the art.

According to some embodiments, the amino acid sequence of the TCR is modified to comprise a detectable label, a therapeutic agent or pharmacokinetic modifying moiety.

Non-limiting examples for detectable labels are radiolabels, fluorescent labels, nucleic acid probes, enzymes and contrast reagents. Therapeutic agents which may be associated with the TCRs include radioactive compounds, immunomodulators, enzymes or chemotherapeutic agents. The therapeutic agents could be enclosed by a liposome linked to TCR so that the compound can be released slowly at the target site. This will avoid damage during the transport in the body and ensure that the therapeutic agent, e.g., toxin, exerts its maximum effect after binding of the TCR to the relevant antigen presenting cells. Other examples for therapeutic agents are: peptide cytotoxins, i.e. proteins or peptides with the ability to kill mammalian cells, such as ricin, diphtheria toxin, pseudomonas bacterial exotoxin A, DNase and RNase. Small molecule cytotoxic agents, i.e., compounds with the ability to kill mammalian cells having a molecular weight of less than 700 Daltons. Such compounds could contain toxic metals capable of having a cytotoxic effect. Furthermore, it is to be understood that these small molecule cytotoxic agents also include pro-drugs, i.e., compounds that decay or are converted under physiological conditions to release cytotoxic agents. Such agents may for example include docetaxel, gemcitabine, cis-platin, maytansine derivatives, rachelmycin, calicheamicin, etoposide, ifosfamide, irinotecan, porfimer sodium photofrin II, temozolomide, topotecan, trimetrexate glucoronate, mitoxantrone, auristatin E, vincristine and doxorubicin; radionuclides, such as, iodine 131, rhenium 186, indium 111, yttrium 90. bismuth 210 and 213, actinium 225 and astatine 213. The association of the radionuclides with the TCRs or derivatives thereof may for example be carried out by chelating agents; immunostimulators, also known as immunostimulants, i.e., immune effector molecules which stimulate immune response. Exemplary immunostimulators are cytokines such as IL-2 and IFN-γ, antibodies or fragments thereof, including anti-T cell or NK cell determinant antibodies (e.g., anti-CD3, anti-CD28 or anti-CD16); alternative protein scaffolds with antibody like binding characteristics; superantigens, i.e., antigens that cause non-specific activation of T-cells resulting in polyclonal T cell activation and massive cytokine release, and mutants thereof; chemokines such as IL-8, platelet factor 4, melanoma growth stimulatory protein, etc. complement activators; xenogeneic protein domains, allogeneic protein domains, viral/bacterial protein domains, viral/bacterial peptides.

The antigen receptor molecules (T cell receptor molecules) on human T lymphocytes are non-covalently associated with the CD3 (T3) molecular complex on the cell surface. Perturbation of this complex with anti-CD3 monoclonal antibodies induces T cell activation. Thus, some embodiments refer to a TCR as described herein associated (usually by fusion to a N-or C-terminus of the alpha or beta chain) with an anti-CD3 antibody, or a functional fragment or variant of said anti-CD3 antibody. Antibody fragments and variants/analogues which are suitable for use in the compositions and methods described herein include minibodies, Fab fragments, F(ab′)₂ fragments, dsFv and scFv fragments, Nanobodies™ (Ablynx (Belgium)), molecules comprising synthetic single immunoglobulin variable heavy chain domain derived from a camelid (e.g., camel or llama or alpaca) antibody) and Domain Antibodies (comprising an affinity matured single immunoglobulin variable heavy chain domain or immunoglobulin variable light chain domain (Domantis (Belgium)) or alternative protein scaffolds that exhibit antibody-like binding characteristics such as Affibodies (comprising engineered protein A scaffold Affibody (Sweden)) or Anticalins (comprising engineered anticalins Pieris (Germany)).

The therapeutic agent may preferably be selected from the group consisting of an immune effector molecule, a cytotoxic agent and a radionuclide. Preferably, the immune effector molecule is a cytokine.

The pharmacokinetic modifying moiety may be for example at least one polyethylene glycol repeating unit, at least one glycol group, at least one sialyl group or a combination thereof. The association of at least one polyethylene glycol repeating unit, at least one glycol group, at least one sialyl group may be caused in a number of ways known to those skilled in the art. In a preferred embodiment, the units are covalently linked to the TCR. The TCRs contemplated herein can be modified by one or several pharmacokinetic modifying moieties. In particular, the soluble form of the TCR is modified by one or several pharmacokinetic modifying moieties. The pharmacokinetic modifying moiety may achieve beneficial changes to the pharmacokinetic profile of the therapeutic, for example improved plasma half-life, reduced or enhanced immunogenicity, and improved solubility.

The TCR contemplated herein may be soluble or membrane bound. The term “soluble” refers to a TCR being in soluble form (i.e., having no transmembrane or cytoplasmic domains), for example for use as a targeting agent for delivering therapeutic agents to the antigen presenting cell. For stability, soluble αβ heterodimeric TCRs preferably have an introduced disulfide bond between residues of the respective constant domains, as described, for example, in WO 03/020763. One or both of the constant domains present in an αβ heterodimer may be truncated at the C terminus or C termini, for example by up to 15, or up to 10 or up to 8 or fewer amino acids. For use in adoptive therapy, an αβ heterodimeric TCR may, for example, be transfected as full-length chains having both cytoplasmic and transmembrane domains. TCRs may contain a disulfide bond corresponding to that found in nature between the respective alpha and beta constant domains, additionally or alternatively a non-native disulfide bond may be present.

The TCR, in particular a soluble form of the TCR contemplated herein can thus be modified by attaching additional functional moieties, e.g., for reducing immunogenicity, increasing hydrodynamic size (size in solution) solubility and/or stability (e.g., by enhanced protection to proteolytic degradation) and/or extending serum half-life. Other useful functional moieties and modifications include “suicide” or “safety switches” that can be used to shut off effector host cells carrying an inventive TCR in a patient's body. An example is the inducible Caspase 9 (iCasp9) “safety switch” described by Gargett and Brown. Front Pharmacol 2014; 5:235. Briefly, effector host cells are modified by well-known methods to express a Caspase 9 domain whose dimerization depends on a small molecule dimerizer drug such as AP1903/CIP, and results in rapid induction of apoptosis in the modified effector cells. The system is for instance described in EP2173869 (A2). Examples for other “suicide” or “safety switches” are known in the art, e.g., Herpes Simplex Virus thymidine kinase (HSV-TK), expression of CD20 and subsequent depletion using anti-CD20 antibody or myc tags (Kieback et al. Proc Natl Acad Sci USA 2008 15;105(2): 623-628).

TCRs with an altered glycosylation pattern are also envisaged herein. As is known in the art, glycosylation patterns can depend on the amino acid sequence (e.g., the presence or absence of particular glycosylation amino acid residues, discussed below) and/or the host cell or organism in which the protein is produced. Glycosylation of polypeptides is typically either N-linked or O-linked. N-linked refers to the attachment of the carbohydrate moiety to the side chain of an asparagine residue. Addition of N-linked glycosylation sites to the binding molecule is conveniently accomplished by altering the amino acid sequence such that it contains one or more tri-peptide sequences selected from asparagine-X-serine and asparagine-X-threonine (where X is any amino acid except proline). O-linked glycosylation sites may be introduced by the addition of or substitution by, one or more serine or threonine residues to the starting sequence.

Another means of glycosylation of TCRs is by chemical or enzymatic coupling of glycosides to the protein. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine. Similarly, deglycosylation (i.e., removal of carbohydrate moieties present on the binding molecule) may be accomplished chemically, e.g., by exposing the TCRs to trifluoromethanesulfonic acid, or enzymatically by employing endo-and exo-glycosidases.

It is also conceivable to add a drug such as a small molecule compound to the TCR, in particular a soluble form of the inventive TCR. Linkage can be achieved via covalent bonds, or non-covalent interactions such as through electrostatic forces. Various linkers, known in the art, can be employed in order to form the drug conjugates.

The TCR, in particular a soluble form of the inventive TCR, can additionally be modified to introduce additional domains which aid in identification, tracking, purification and/or isolation of the respective molecule (tags). Thus in some embodiments, the TCR α chain or the TCR β chain may be modified to comprise an epitope tag.

Epitope tags are useful examples of tags that can be incorporated into the TCR. Epitope tags are short stretches of amino acids that allow for binding of a specific antibody and therefore enable identification and tracking of the binding and movement of soluble TCRs or host cells within the patient's body or cultivated (host) cells. Detection of the epitope tag, and hence, the tagged TCR, can be achieved using a number of different techniques. Tags can further be employed for stimulation and expansion of host cells carrying an inventive TCR by cultivating the cells in the presence of binding molecules (antibodies) specific for said tag.

In general, the TCR can be modified in some instances with various mutations that modify the affinity and the off-rate of the TCR with the target antigen. In particular, the mutations may increase the affinity and/or reduce the off-rate. Thus, the TCR may be mutated in at least one CDR and the variable domain framework region thereof.

However, in a preferred embodiment the CDR regions of the TCR are not modified or in vitro affinity maturated such as for the TCR receptors in the examples. This means that the CDR regions have naturally occurring sequences. This can be advantageous, since in vitro affinity maturation may lead to immunogenicity to the TCR molecule. This may lead to the production of anti-drug antibodies decreasing or inactivating the therapeutic effect and the treatment and/or induce adverse effects.

The mutation may be one or more substitution(s), deletion(s) or insertions(s). These mutations may be introduced by any suitable method known in the art, such as DNA synthesis, polymerase chain reaction, restriction enzyme-based cloning, ligation independent cloning procedures, which are described for example in Sambrook, Cold Spring Harbor Laboratory Press 2012.

Theoretically, unpredictable TCR specificity with the risk for cross-reactivity can occur due to mispairing between endogenous and exogenous TCR chains. To avoid mispairing of TCR sequences, the recombinant TCR sequence may be modified to contain minimal murinized Cα and Cβ regions, a technology that has been shown to efficiently enhance correct pairing of several different transduced TCR chains. Murinization of TCRs (i.e. exchanging the human constant regions in the alpha and beta chain by their murine counterparts) is a technique that is commonly applied in order to improve cell surface expression of TCRs in host cells. Without wishing to be bound by specific theory, it is thought that murinized TCRs associate more effectively with CD3 co-receptors; and/or that preferentially pair with each other and are less prone to form mixed TCRs on human T cells genetically modified ex vivo to express the TCRs of desired antigenic specificity, but still retaining and expressing their “original” TCRs.

Nine amino acids responsible for the improved expression of murinized TCRs have been identified (Sommermeyer and Uckert, J Immunol. 2010; 184(11):6223-6231) and it is envisaged to substitute one or all of the amino acid residues in the TCRs alpha and/or beta chain constant region for their murine counterpart residues. This technique is also referred to as “minimal murinization” and offers the advantage of enhancing cell surface expression while, at the same time, reducing the number of “foreign” amino acid residues in the amino acid sequence and, thereby, the risk of immunogenicity.

In a preferred embodiment the TCRs containing minimal murinized Cα and Cβ regions are TCR-1 comprising the a chain of SEQ ID NO: 10 and the β chain of SEQ ID NO: 11, TCR-2 comprising the a chain of SEQ ID NO: 20 and the β chain of SEQ ID NO: 21, TCR-3 comprising the a chain of SEQ ID NO: 30 and the β chain of SEQ ID NO: 31.

In preferred embodiments, the TCRs contain minimally murinized Cα and Cβ regions and further comprise hydrophobic amino acid mutations in the Cα transmembrane domain. The transmembrane domain of the TCR α chain has been shown to contribute to the lack of stability of the whole chain and thereby affecting the formation and surface expression of the whole TCR-CD3 complex. Substitution of three amino acids in the TCR α transmembrane domain with the hydrophobic amino acids leucine or valine increased TCR expression and functional avidity. Haga-Friedman et al. J. Immunology 2012; 188:5538-5546.

In a preferred embodiment the TCRs containing minimally murinized Cα and Cβ regions and hydrophobic amino acid substitutions in the TCR α chain are TCR-7 comprising the α chain of SEQ ID NO: 102 and the β chain of SEQ ID NO: 103, TCR-8 comprising the α chain of SEQ ID NO: 108 and the β chain of SEQ ID NO: 109, TCR-9 comprising the α chain of SEQ ID NO: 114 and the β chain of SEQ ID NO: 115.

Some embodiments refer to an isolated TCR as described herein, wherein the TCR is of the single chain type, wherein the TCR α chain and the TCR β chain are linked by a linker sequence, optionally wherein the linker is cleavable.

A suitable single chain TCR form comprises a first segment constituted by an amino acid sequence corresponding to a variable TCR α region, a second segment constituted by an amino acid sequence corresponding to a variable TCR β region fused to the N terminus of an amino acid sequence corresponding to a TCR β chain constant region extracellular sequence, and a linker sequence linking the C terminus of the first segment to the N terminus of the second segment. Alternatively, the first segment may be constituted by an amino acid sequence corresponding to a TCR β chain variable region, the second segment may be constituted by an amino acid sequence corresponding to a TCR α chain variable region sequence fused to the N terminus of an amino acid sequence corresponding to a TCR α chain constant region extracellular sequence. The above single chain TCRs may further comprise a disulfide bond between the first and second chains, and wherein the length of the linker sequence and the position of the disulfide bond being such that the variable domain sequences of the first and second segments are mutually orientated substantially as in native T cell receptors. More specifically, the first segment may be constituted by an amino acid sequence corresponding to a TCR α chain variable region sequence fused to the N terminus of an amino acid sequence corresponding to a TCR α chain constant region extracellular sequence, the second segment may be constituted by an amino acid sequence corresponding to a TCR β chain variable region fused to the N terminus of an amino acid sequence corresponding to TCR β chain constant region extracellular sequence, and a disulfide bond may be provided between the first and second chains. The linker sequence may be any sequence which does not impair the function of the TCR.

A “functional” TCR α and/or β chain fusion protein shall mean a TCR or TCR variant, for example modified by addition, deletion or substitution of amino acids, that maintains at least substantial biological activity. In the case of the α and/or β chain of a TCR, this shall mean that both chains remain able to form a T-cell receptor (either with a non-modified α and/or β chain or with another inventive fusion protein α and/or β chain) which exerts its biological function, in particular binding to the specific peptide-MHC complex of said TCR, and/or functional signal transduction upon specific peptide: MHC interaction.

In specific embodiments, the TCR may be modified, to be a functional T-cell receptor (TCR) α and/or β chain fusion protein, wherein said epitope-tag has a length of between 6 to 15 amino acids, preferably 9 to 11 amino acids. In another embodiment the TCR may be modified to be a functional T-cell receptor (TCR) α and/or β chain fusion protein wherein said T-cell receptor (TCR) α and/or β chain fusion protein comprises two or more epitope-tags, either spaced apart or directly in tandem. Embodiments of the fusion protein can contain 2, 3, 4, 5 or even more epitope-tags, as long as the fusion protein maintains its biological activity/activities (“functional”).

Preferred is a functional T-cell receptor (TCR) α and/or β chain fusion protein according to the present invention, wherein said epitope-tag is selected from, but not limited to, CD20 or Her2/neu tags, or other conventional tags such as a myc-tag, FLAG-tag, T7-tag, HA (hemagglutinin)-tag, His-tag, S-tag, GST-tag, or GFP-tag. Myc-, T7-, GST-, GFP-tags are epitopes derived from existing molecules. In contrast, FLAG is a synthetic epitope tag designed for high antigenicity (see, e.g., U.S. Pat. Nos. 4,703,004 and 4,851,341). The myc tag can preferably be used because high quality reagents are available to be used for its detection. Epitope tags can of course have one or more additional functions, beyond recognition by an antibody. The sequences of these tags are described in the literature and well known to the person of skill in art.

In more preferred embodiments, an isolated TCR is expressed as a fusion protein, wherein the TCR α chain and the TCR β chain are separated by one or more polypeptide cleavage signal signals. In particular embodiments, a fusion protein comprises from 5′ to 3′: a TCR α chain, one or more polypeptide cleavage signal signals, and a TCR β chain. In particular embodiments, a fusion protein comprises from 5′ to 3′: a TCR β chain, one or more polypeptide cleavage signal signals, and a TCR α chain.

Polypeptide cleavage signals contemplated herein include, but are not limited to, protease cleavage sites and ribosomal skip sequences. A polypeptide cleavage signal may be disposed between each of the polypeptide domains described herein, e.g., a TCR α chain and a TCR β chain. In addition, a polypeptide cleavage signal can be put into any linker peptide sequence. Exemplary polypeptide cleavage signals include polypeptide cleavage recognition sites such as protease cleavage sites, nuclease cleavage sites (e.g., rare restriction enzyme recognition sites, self-cleaving ribozyme recognition sites), and self-cleaving viral oligopeptides or ribosomal skipping sequences (see deFelipe and Ryan, 2004. Traffic, 5(8); 616-26).

Illustrative examples of protease cleavage sites suitable for use in particular embodiments include but are not limited to furin (e.g., Arg-X-X-Arg, such as Arg-X-Lys/Arg-Arg or Arg-GIn/Tyr-Lys/Arg-Arg. Furin may further cleave the sequences Arg-Ala-Arg-Tyr-Lys-Arg or Arg-Ala-Arg-Tyr-Lys-Arg-Ser); subtilisins (e.g., PC2, PC1/PC3, PACE4, PC4, PC5/PC6, LPC/PC7IPC8/SPC7 and SKI-I); enterokinase (e.g., Asp-Asp-Asp-Aps-Lys* and Asp/Glu-Arg-* Met); factor Xa (e.g., Glu-Gly-Arg*); thrombin (e.g., Leu-Val-Pro-Arg*Gly-Ser); Granzyme B (e.g., Ile-Glu-Pro-Asp*); Caspase-3 (e.g., Asp-Glu-Val-Asp*); and the like.

Illustrative examples of self-cleaving viral peptides or ribosomal skipping sequences include but are not limited to a 2A or 2A-like site, sequence or domain (Donnelly et al., 2001. J. Gen. Virol. 82:1027-1041). In particular embodiments, the viral 2A peptide is an aphthovirus 2A peptide, a potyvirus 2A peptide, or a cardiovirus 2A peptide. In preferred embodiments, the viral 2A peptide is selected from the group consisting of: a foot-and-mouth disease virus 2A peptide (F2A), an equine rhinitis A virus 2A peptide (E2A), a Thosea asigna virus 2A peptide (T2A), a porcine teschovirus-1 2A peptide (P2A), a Theilovirus 2A peptide, and an encephalomyocarditis virus 2A peptide.

In certain embodiments, a fusion protein comprises a TCR α chain, a proteolytic cleavage site and/or a ribosomal skip sequence and a TCR β chain. In preferred embodiments, the fusion protein comprises a TCR α chain, a furin cleavage site and/or a P2A ribosomal skip sequence and a TCR β chain. In other preferred embodiments, the fusion protein comprises a TCR α chain, a P2A ribosomal skip sequence, and a TCR β chain.

In particular embodiments, a fusion protein comprises a TCR β chain, a proteolytic cleavage site and/or a ribosomal skip sequence and a TCR α chain. In preferred embodiments, the fusion protein comprises a TCR β chain, a furin cleavage site and/or a P2A ribosomal skip sequence and a TCR α chain. In other preferred embodiments, the fusion protein comprises a TCR β chain, a P2A ribosomal skip sequence, and a TCR α chain.

In preferred embodiments, fusion proteins comprise an amino acid sequence set forth in any one of SEQ ID NOs: 94, 96, 98, 104, 110, and 116.

### F. TCR Fragments and Variants

Another aspect refers to a polypeptide comprising a functional portion of the TCR of as described herein. The functional portion may comprise at least one of the amino acid sequences selected from the group consisting of SEQ ID NO: 4, SEQ ID NO: 14, SEQ ID NO: 24, SEQ ID NO: 7, SEQ ID NO: 17, SEQ ID NO: 27.

In specific embodiments the polypeptide may the functional portion of the TCR alone, e.g., in a soluble form. Alternatively, the polypeptide may be combined with other domains.

The functional portion may mediate the binding of the TCR to the antigen, in particular to the antigen-MHC complex. In one embodiment, the functional portion comprises the TCR α variable chain and/or the TCR β variable chain as described herein.

The TCR variant molecule, i.e., a molecule combining a polypeptide comprising a functional portion of the TCR with other domains, may have the binding properties of the TCR receptor but may be combined with signaling domains of effectors cells (other than T cells), in particular with signaling domains of NK cells. Therefore, some embodiments refer to a protein comprising a functional portion of the TCR as described herein in combination with the signaling domains of an immune effector cell, such as a NK cell.

“Binding” refers to the ability to specifically and non-covalently associate, unite or bind with the target.

Another aspect refers to a multivalent TCR complex comprising at least two TCRs as described herein. In one embodiment of this aspect, at least two TCR molecules are linked via linker moieties to form multivalent complexes. Preferably, the complexes are water soluble, so the linker moiety should be selected accordingly. It is preferable that the linker moiety is capable of attaching to defined positions on the TCR molecules, so that the structural diversity of the complexes formed is minimized. One embodiment of the present aspect is provided by a TCR complex wherein the polymer chain or peptidic linker sequence extends between amino acid residues of each TCR, which are not located in a variable region sequence of the TCR. Since the complexes may be for use in medicine, the linker moieties should be chosen with due regard to their pharmaceutical suitability, for example their immunogenicity. Examples of linker moieties, which fulfil the above desirable criteria are known in the art, for example the art of linking antibody fragments.

Examples for linkers are hydrophilic polymers and peptide linkers. An example for hydrophilic polymers are polyalkylene glycols. The most commonly used of this class are based on polyethylene glycol or PEG. However, others are based on other suitable, optionally substituted, polyalkylene glycols which include polypropylene glycol, and copolymers of ethylene glycol and propylene glycol. Peptide linkers are comprised of chains of amino acids, and function to produce simple linkers or multimerization domains onto which TCR molecules can be attached.

One embodiment refers to a multivalent TCR complex, wherein at least one of said TCRs is associated with a therapeutic agent.

### G. Cytokine and Chemokine Release

Some embodiments refer to the isolated TCR as described herein, polypeptide as described herein, multivalent TCR complex as described herein, wherein IFN-γ secretion is induced by binding of the inventive TCR expressed on an effector cell to the amino acid sequence of SEQ ID NOs: 1 which is presented by the HLA-A*02:01 encoded molecule.

The IFN-γ secretion induced by binding of the inventive TCR expressed on an effector cell to the amino acid sequence of SEQ ID NO: 1, which is presented by the HLA-A*02:01 encoded molecule, may be more than 100 times higher, preferably 500 times higher, more preferably 2000 times higher when binding to the amino acid sequence of SEQ ID NO: 1, which is presentedby the HLA-A*02:01 encoded molecule, compared to binding to an irrelevant peptide (ASTN1, SEQ ID NO. 56, KLYGLDWAEL), which is presented by the HLA-A*02:01 encoded molecule. The IFN-γ secretion may be for example more than 100 pg/ml, such as more than 500 pg/ml or more than 2000 pg/ml.

The cytokine and chemokine release, such as IFN-γsecretion may be measured using an in vitro assay in which K562 cells (Greiner et al. 2006, Blood. 2006 Dec. 15;108(13):4109-17) are transfected with ivtRNA or transduced to express the amino acid sequence of SEQ ID NO: 1 or irrelevant peptide, respectively, and are incubated with CD8+ enriched and/or non-CD8+-enriched PBMCs expressing the TCR to be investigated or in an in vitro assay using T2 cells externally loaded with either the SEQ ID NO: 1 or the irrelevant peptide and subsequently co-incubated with CD8+ enriched and/or non-CD8+-enriched PBMCs expressing the TCR to be investigated.

Some embodiments refer to an isolated TCR as described herein, polypeptide as described herein or multivalent TCR complex as described herein, wherein IFN-α secretion induced by binding of the inventive TCR expressed on an effector cell to the amino acid sequence of SEQ ID NO: 1 or in particular to the amino acid sequence of SEQ ID NO: 1 which is presented by the HLA-A*02:01 encoded molecule is below a predefined threshold. The threshold may be determined by using a specific effector to target ratio of at least 2:1.

The “effector cell” may be a peripheral blood lymphocyte (PBL) or a peripheral blood mononuclear cell (PBMC). Typically, the effector cell is an immune effector cell, especially a T cell. Other suitable cell types include gamma-delta T cells, natural killer (NK) cells, and NK-like T (NKT) cells.

The IFN-γ secretion upon binding of the inventive TCR expressed on an effector cell to amino acid sequence of SEQ ID NO: 1 which is presented by the HLA-A*02:01 encoded molecule may be induced at a MAGE-A4 peptide concentration of at least 10⁻⁷ [M], preferably at least 10⁻⁸ [M], more preferably 10⁻⁹ [M]. In specific embodiments, for example when the ratio of TCR-transgenic T cells to T2 cells is 2:1, the IFN-γ secretion upon by binding of the inventive TCR expressed on an effector cell to amino acid sequence of SEQ ID NO: 1 which is presented by the HLA-A*02:01 encoded molecule may be induced at a MAGE-A4 peptide concentration of at least 10⁻⁷ [M], preferably at least 10⁻⁸ [M], more preferably 10⁻⁹ [M].

The invention relates also to methods for identifying a TCR or a fragment thereof that binds to the target amino acid sequence SEQ ID NO: 1, which is presented by the HLA-A*02:01 encoded molecule, wherein the method comprises contacting the candidate TCR or fragment thereof with the amino acid sequence SEQ ID NO: 1, which is presented by the HLA-A*02:01 encoded molecule, and determining whether the candidate TCR or fragment thereof binds to the target and/or mediates an immune response.

Whether the candidate TCR or fragment thereof mediates an immune response can be determined for example by the measurement of cytokine secretion, such as IFN-γsecretion. As described above cytokine secretion may be e.g., measured by an in vitro assay in which K562 cells (or other APCs) transfected with ivtRNA coding the amino acid sequence SEQ ID NO: 1 are incubated with CD8+ enriched PBMC expressing the TCR or a molecule comprising a fragment of the TCR to be investigated.

### H. Nucleic Acids, Vectors

Another aspect refers to a nucleic acid encoding a TCR as described herein or encoding the polynucleotide encoding a TCR as described herein.

The following table indicates the nucleotide sequences encoding the respective peptide sequences:

| Polypeptide | Polynucleotide | |
|---|---|---|
| SEQ ID NO | SEQ ID NO | Description |
| 2 | 32 | TCR-1 α chain CDR1 |
| 3 | 33 | TCR-1 α chain CDR2 |
| 4 | 34 | TCR-1 α chain CDR3 |
| 5 | 35 | TCR-1 β chain CDR1 |
| 6 | 36 | TCR-1 β chain CDR2 |
| 7 | 37 | TCR-1 β chain CDR3 |
| 10 | 38 | TCR-1 α chain complete |
| 11 | 39 | TCR-1 β chain complete |
| 12 | 40 | TCR-2 α chain CDR1 |
| 13 | 41 | TCR-2 α chain CDR2 |
| 14 | 42 | TCR-2 α chain CDR3 |
| 15 | 43 | TCR-2 β chain CDR1 |
| 16 | 44 | TCR-2 β chain CDR2 |
| 17 | 45 | TCR-2 β chain CDR3 |
| 20 | 46 | TCR-2 α chain complete |
| 21 | 47 | TCR-2 β chain complete |
| 22 | 48 | TCR-3 α chain CDR1 |
| 23 | 49 | TCR-3 α chain CDR2 |
| 24 | 50 | TCR-3 α chain CDR3 |
| 25 | 51 | TCR-3 β chain CDR1 |
| 26 | 52 | TCR-3 β chain CDR2 |
| 27 | 53 | TCR-3 β chain CDR3 |
| 30 | 54 | TCR-3 α chain complete |
| 31 | 55 | TCR-3 β chain complete |
| 2 | 63 | TCR-4 α chain CDR1 |
| 3 | 64 | TCR-4 α chain CDR2 |
| 4 | 65 | TCR-4 α chain CDR3 |
| 5 | 66 | TCR-4 β chain CDR1 |
| 6 | 67 | TCR-4 β chain CDR2 |
| 7 | 68 | TCR-4 β chain CDR3 |
| 87 | 69 | TCR-4 α chain complete |
| 88 | 70 | TCR-4 β chain complete |
| 12 | 71 | TCR-5 α chain CDR1 |
| 13 | 72 | TCR-5 α chain CDR2 |
| 14 | 73 | TCR-5 α chain CDR3 |
| 15 | 74 | TCR-5 β chain CDR1 |
| 16 | 75 | TCR-5 β chain CDR2 |
| 17 | 76 | TCR-5 β chain CDR3 |
| 89 | 77 | TCR-5 α chain complete |
| 90 | 78 | TCR-5 β chain complete |
| 22 | 79 | TCR-6 α chain CDR1 |
| 23 | 80 | TCR-6 α chain CDR2 |
| 24 | 81 | TCR-6 α chain CDR3 |
| 25 | 82 | TCR-6 β chain CDR1 |
| 26 | 83 | TCR-6 β chain CDR2 |
| 27 | 84 | TCR-6 β chain CDR3 |
| 91 | 85 | TCR-6 α chain complete |
| 92 | 86 | TCR-6 β chain complete |
| 94 | 93 | TCR-4 fusion protein |
| 96 | 95 | TCR-5 fusion protein |
| 98 | 97 | TCR-6 fusion protein |
| 102 | 99 | TCR-7 α chain complete |
| 103 | 100 | TCR-7 β chain complete |
| 104 | 101 | TCR-7 fusion protein |
| 108 | 105 | TCR-8 α chain complete |
| 109 | 106 | TCR-8 β chain complete |
| 110 | 107 | TCR-8 fusion protein |
| 114 | 108 | TCR-9 α chain complete |
| 115 | 109 | TCR-9 β chain complete |
| 116 | 110 | TCR-9 fusion protein |

“Nucleic acid molecule” and “nucleotide sequence” generally means a polymer of DNA or RNA, which can be single-stranded or double-stranded, synthesized or obtained (e.g., isolated and/or purified) from natural sources, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide. Preferably, the nucleic acids described herein are recombinant. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication. The nucleic acids can be constructed based on chemical synthesis and/or enzymatic ligation reactions using procedures known in the art or commercially available (e.g., from Genscript, Thermo Fisher and similar companies). For example, a nucleic acid can be chemically synthesized (see Sambrook et al.) using naturally occurring nucleotides or variously modified nucleotides designed to increase the biological stability of the molecules or to increase the physical stability of the duplex formed upon hybridization (e.g., phosphorothioate derivatives and acridine-substituted nucleotides). The nucleic acid can comprise any nucleotide sequence which encodes any of the recombinant TCRs, polypeptides, or proteins, or functional portions or functional variants thereof.

The present disclosure also provides variants of the isolated or purified nucleic acids wherein the variant nucleic acids comprise a nucleotide sequence that has at least 75%, 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence encoding the TCR described herein. Such variant nucleotide sequence encodes a functional TCR that specifically recognizes MAGE-A4.

The disclosure also provides an isolated or purified nucleic acid comprising a nucleotide sequence, which is complementary to the nucleotide sequence of any of the nucleic acids described herein or a nucleotide sequence which hybridizes under stringent conditions to the nucleotide sequence of any of the nucleic acids described herein.

The nucleotide sequence, which hybridizes under stringent conditions preferably hybridizes under high stringency conditions. By “high stringency conditions” is meant that the nucleotide sequence specifically hybridizes to a target sequence (the nucleotide sequence of any of the nucleic acids described herein) in an amount that is detectably stronger than non-specific hybridization. High stringency conditions include conditions which would distinguish a polynucleotide with an exact complementary sequence, or one containing only a few scattered mismatches from a random sequence that happened to have a few small regions (e.g., 3-10 bases) that matched the nucleotide sequence. Such small regions of complementarity are more easily melted than a full-length complement of 14-17 or more bases, and high stringency hybridization makes them easily distinguishable. Relatively high stringency conditions would include, for example, low salt and/or high temperature conditions, such as provided by about 0.02-0.1 M NaCl or the equivalent, at temperatures of about 50-70° C. Such high stringency conditions tolerate little, if any, mismatch between the nucleotide sequence and the template or target strand and are particularly suitable for detecting expression of any of the TCRs described herein. It is generally appreciated that conditions can be rendered more stringent by the addition of increasing amounts of formamide.

In particular embodiments, nucleic acids are codon optimized. As used herein, the term “codon-optimized” refers to substituting codons in a polynucleotide encoding a polypeptide in order to increase the expression, stability and/or activity of the polypeptide. Factors that influence codon optimization include, but are not limited to one or more of: (i) variation of codon biases between two or more organisms or genes or synthetically constructed bias tables, (ii) variation in the degree of codon bias within an organism, gene, or set of genes, (iii) systematic variation of codons including context, (iv) variation of codons according to their decoding tRNAs, (v) variation of codons according to GC %, either overall or in one position of the triplet, (vi) variation in degree of similarity to a reference sequence for example a naturally occurring sequence, (vii) variation in the codon frequency cutoff, (viii) structural properties of mRNAs transcribed from the DNA sequence, (ix) prior knowledge about the function of the DNA sequences upon which design of the codon substitution set is to be based, (x) systematic variation of codon sets for each amino acid, and/or (xi) isolated removal of spurious translation initiation sites.

Another embodiment refers to a vector comprising the nucleic acid encoding the TCR as described herein. The vector is preferably a plasmid, shuttle vector, phagemid, cosmid, expression vector, retroviral vector, adenoviral vector or particle and/or vector to be used in gene therapy.

A “vector” is any molecule or composition that has the ability to carry a nucleic acid sequence into a suitable host cell where synthesis of the encoded polypeptide can take place. Typically, and preferably, a vector is a nucleic acid that has been engineered, using recombinant DNA techniques that are known in the art, to incorporate a desired nucleic acid sequence. The vector may comprise DNA or RNA and/or comprise liposomes. The vector may be a plasmid, shuttle vector, phagemid, cosmid, expression vector, retroviral vector, lentiviral vector, adenoviral vector or particle and/or vector to be used in gene therapy. A vector may include nucleic acid sequences that permit it to replicate in a host cell, such as an origin of replication. A vector may also include one or more selectable marker genes and other genetic elements known to those of ordinary skill in the art. A vector preferably is an expression vector that includes a nucleic acid according to the present invention operably linked to sequences allowing for the expression of said nucleic acid.

In preferred embodiments, a vector comprises a nucleic acid encoding a TCR β chain having an amino acid sequence set forth in SEQ ID NO: 88 and a TCR α chain having an amino acid sequence set forth in SEQ ID NO: 87; a TCR β chain having an amino acid sequence set forth in SEQ ID NO: 90 and a TCR α chain having an amino acid sequence set forth in SEQ ID NO: 89; a TCR β chain having an amino acid sequence set forth in SEQ ID NO: 92 and a TCR α chain having an amino acid sequence set forth in SEQ ID NO: 91; a TCR β chain having an amino acid sequence set forth in SEQ ID NO: 103 and a TCR α chain having an amino acid sequence set forth in SEQ ID NO: 102; a TCR β chain having an amino acid sequence set forth in SEQ ID NO: 109 and a TCR α chain having an amino acid sequence set forth in SEQ ID NO: 108; or a TCR β chain having an amino acid sequence set forth in SEQ ID NO: 115 and a TCR α chain having an amino acid sequence set forth in SEQ ID NO: 114.

Preferably, the vector is an expression vector. More preferably, the vector is a retroviral, more specifically a gamma-retroviral or lentiviral vector.

### I. Cells, Cell Lines

Another aspect refers to a cell expressing the TCR as described herein. In some embodiments, the cell is isolated or non-naturally occurring. In specific embodiments, the cell may comprise the nucleic acid encoding the TCR as described herein or the vector comprising said nucleic acid.

In the cell, the above described vector comprising a nucleic acid sequence coding for the above described TCR may be introduced or ivtRNA coding for said TCR may be introduced. The cell may be a peripheral blood lymphocyte such as a T cell. The method of cloning and exogenous expression of the TCR is for example described in Engels et al. Cancer Cell, 2013; 23(4): 516-526.The transduction of primary human T cells with a lentiviral vector is, for example, described in Cribbs et al. BMC Biotechnol. 2013; 13:98.

The term “transfection” refers to a non-viral process by which an exogenous nucleic acid sequence is introduced in a host cell, e.g., in an eukaryotic host cell. It is noted that introduction or transfer of nucleic acid sequences is not limited to the mentioned methods but can be achieved by any number of means including electroporation, microinjection, gene gun delivery, lipofection, or superfection.

The term “transduction” refers to the introduction of an exogenous nucleic acid sequence into a host cell using a viral vector, e.g., an adenovirus, an adeno-associated virus (AAV), a vaccinia virus, a herpes virus, a retrovirus, or lentivirus.

Some embodiments refer to a cell comprising: a) an expression vector which comprises at least one nucleic acid as described herein, or b) a first expression vector which comprises a nucleic acid encoding the alpha chain of the TCR as described herein, and a second expression vector which comprises a nucleic acid encoding the beta chain of a TCR as described herein.

In some embodiments, the cell is a peripheral blood lymphocyte (PBL) or a peripheral blood mononuclear cell (PBMC). The cell may be a natural killer (NK) cell, natural killer like T (NKT) cell or a T cell. Preferably, the cell is a T cell. The T cell may be a CD4+ or a CD8+ T cell or double negative T cells, i.e., T cells expressing neither CD4 or CD8. In some embodiments, the cell is a stem cell like memory T cell.

In preferred embodiments, the TCR functions independently of co-receptors, i.e., the TCR is function in both CD8+ and CD4+ cells, e.g., TCR-5 and TCR-8.

Stem cell-like memory T cells (TSCM) are a less-differentiated subpopulation of CD8+T cells, which are characterized by the capacity of self-renewal and to persist long-term. Once these cells encounter their antigen in vivo, they differentiate further into central memory T cells (TCM), effector memory T cells (TEM) and terminally differentiated effector memory T cells (TEMRA) with some TSCM remaining quiescent (Flynn et al., Clinical & Translational Immunology 2014; 3(7): e20.). These remaining TSCM cells show the capacity to build a durable immunological memory in vivo and therefore are considered an important T cell subpopulation for adoptive T cell therapy (Lugli et al., Nature Protocols 2013; 8:33-42, Gattinoni et al., Nat. Med. 2011; Oct; 17(10): 1290-1297). Immune-magnetic selection can be used in order to restrict the T cell pool to the stem cell memory T cell subtype see (Riddell et al. Cancer Journal 2014; 20(2): 141-144)

### J. Antibodies Targeting TCR

Another aspect refers to an antibody or antigen-binding fragment thereof specifically binding to a portion of the TCR as described herein that mediates specificity for MAGE-A4. In one embodiment, the portion of the TCR that mediates the MAGE-A4 specificity comprises the CDR3 of the TCR alpha chain selected from the group consisting of SEQ ID NO: 4, SEQ ID NO: 14 and SEQ ID NO: 24 and the CDR3 of the beta chain selected from the group consisting of SEQ ID NO: 7, SEQ ID NO: 17 or SEQ ID NO: 27.

The antibody or antigen-binding fragment thereof may modulate the activity of the TCR. It may block or may not block the binding of the TCR with MAGE-A4. It could be used for modulating the therapeutic activity of the TCR or for diagnostic purposes.

### K. Pharmaceutical Compositions, Medical Treatments and Kits

Another aspect refers to compositions comprising the TCR as described herein, the polypeptide comprising a functional portion of said TCR, the multivalent TCR complex as described herein, the nucleic acid encoding the TCR, the vector comprising said nucleic acid, the cell comprising said TCR, or the antibody specifically binding to a portion of the TCR as described herein.

Another aspect refers to pharmaceutical compositions comprising the TCR as described herein, the polypeptide comprising a functional portion of said TCR, the multivalent TCR complex as described herein, the nucleic acid encoding the TCR, the vector comprising said nucleic acid, the cell comprising said TCR, or the antibody specifically binding to a portion of the TCR as described herein.

Those active components of the present invention are preferably used in such a pharmaceutical composition, in doses mixed with an acceptable carrier or carrier material, that the disease can be treated or at least alleviated. Such a composition can (in addition to the active component and the carrier) include filling material, salts, buffer, stabilizers, solubilizers and other materials, which are known state of the art.

The term “pharmaceutically acceptable” defines a non-toxic material, which does not interfere with effectiveness of the biological activity of the active component. The choice of the carrier is dependent on the application.

The pharmaceutical composition may contain additional components which enhance the activity of the active component or which supplement the treatment. Such additional components and/or factors can be part of the pharmaceutical composition to achieve synergistic effects or to minimize adverse or unwanted effects.

Techniques for the formulation or preparation and application/medication of active components of the present invention are published in Remington: The Science and Practice of Pharmacy, volume I and volume II. 22^{nd} Edition. Edited by Loyd V. Allen Jr. Philadelphia, PA: Pharmaceutical Press; 2012, which is incorporated by reference herein, in its entirety. An appropriate application is a parenteral application, for example intramuscular, subcutaneous, intramedullar injections as well as intrathecal, direct intraventricular, intravenous, intranodal, intraperitoneal or intratumoral injections. The intravenous injection or infusion is the preferred treatment of a patient.

According to a preferred embodiment, the pharmaceutical composition is administered by an infusion or an injection. An injectable composition is a pharmaceutically acceptable fluid composition comprising at least one active ingredient, e.g., an expanded T cell population (for example autologous or allogenic to the patient to be treated) expressing a TCR. The active ingredient is usually dissolved or suspended in a physiologically acceptable carrier, and the composition can additionally comprise minor amounts of one or more non-toxic auxiliary substances, such as emulsifying agents, preservatives, and pH buffering agents and the like. Such injectable compositions that are useful for use with the fusion proteins of this disclosure are conventional; appropriate formulations are well known to those of ordinary skill in the art.

Typically, the pharmaceutical composition comprises at least one pharmaceutically acceptable carrier.

Accordingly, another aspect refers to the TCR as described herein, the polypeptide comprising a functional portion of said TCR, the multivalent TCR complex according as described herein, the nucleic acid encoding said TCR, the vector comprising said nucleic acid, the cell comprising said TCR, the antibody specifically binding to a portion of the TCR, a composition or pharmaceutical composition comprising one or more cells expressing the TCR as described herein for use as a medicament.

Some embodiments refer to the TCR as described herein, the polypeptide comprising a functional portion of said TCR, the multivalent TCR complex according as described herein, the nucleic acid encoding said TCR, the vector comprising said nucleic acid, the cell comprising said TCR, or compositions or pharmaceutical compositions comprising the same for use in the treatment of cancer.

In one embodiment, the cancer is a hematological cancer or a solid tumor. Hematological cancers also called blood cancers, which do not form solid tumors and therefore are dispersed in the body. Examples of hematological cancers are leukemia, lymphoma or multiple myeloma. There are two major types of solid tumors, sarcomas and carcinomas. Sarcomas are for example tumors of the blood vessel, bone, fat tissue, ligament, lymph vessel, muscle or tendon.

In one embodiment, the cancer is selected from the group consisting of sarcoma, prostate cancer, uterine cancer, thyroid cancer, testicular cancer, renal cancer, pancreatic cancer, ovarian cancer, esophageal cancer, non-small-cell lung cancer, (NSCLC) small-cell lung cancer (SCLC), non-Hodgkin's lymphoma, multiple myeloma, melanoma, hepatocellular carcinoma, head and neck cancer, gastric cancer, endometrial cancer, colorectal cancer, cholangiocarcinoma, breast cancer, bladder cancer, myeloid leukemia and acute lymphoblastic leukemia. Preferably, the cancer is selected from the group consisting of NSCLC, SCLC, breast, ovarian or colorectal cancer, or sarcoma. More preferably, the cancer is selected from urothelial (bladder) cancers, melanoma, head and neck cancer, ovarian cancer, NSCLC,esophageal cancer, gastric cancers, synovial sarcoma, and Myxoid Round Cell Liposarcoma (MRCLS).

In one embodiment, the TCR recognize lung cancer cell lines, such as the NSCLC cell line NCI-H1703 and the liver metastases cell line of NSCLC, NCI-H1755.

Also contemplated herein are pharmaceutical compositions and kits containing one or more of (i) an isolated TCR as described herein; (ii) viral particles comprising a nucleic acid encoding a recombinant TCR; (iii) immune cells, such as T cells or NK cells, modified to express a recombinant TCR as described herein; (iv) nucleic acids encoding a recombinant TCR as described herein. In some embodiments, the present disclosure provides compositions comprising lentiviral vector particles comprising a nucleotide sequence encoding a recombinant TCR described herein (or T cells that have been modified using the vector particles described herein to express a recombinant TCR). Such compositions can be administered to subjects in the methods of the present disclosure as described further herein.

Compositions comprising the modified T cells as described herein can be utilized in methods and compositions for adoptive immunotherapy in accordance with known techniques, or variations thereof that will be apparent to those skilled in the art based on the instant disclosure.

In some embodiments, the cells are formulated by first harvesting them from their culture medium, and then washing and concentrating the cells in a medium and container system suitable for administration (a “pharmaceutically acceptable” carrier) in a treatment-effective amount. Suitable infusion medium can be any isotonic medium formulation, typically normal saline, Normosol R (Abbott) or Plasma-Lyte A (Baxter), but also 5% dextrose in water or Ringer's lactate can be utilized. The infusion medium can be supplemented with human serum albumin.

The number of cells for an effective treatment in the composition is typically greater than 10 cells, and up to 10⁶, up to and including 10⁸ or 10⁹ cells and can be more than 10¹⁰ cells. The number of cells will depend upon the ultimate use for which the composition is intended as will the type of cells included therein. For example, if cells that are specific for a particular antigen are desired, then the population will contain greater than 70%, generally greater than 80%, 85% and 90-95% of such cells. For uses provided herein, the cells are generally in a volume of a liter or less, can be 500 ml or less, even 250 ml or 100 ml or less. Hence the density of the desired cells is typically greater than 10⁶ cells/ml and generally is greater than 10⁷ cells/ml, generally 10⁸ cells/ml or greater. The clinically relevant number of immune cells can be apportioned into multiple infusions that cumulatively equal or exceed 10⁹, 10¹⁰ or 10¹¹ cells.

Pharmaceutical compositions provided herein can be in various forms, e.g., in solid, liquid, powder, aqueous, or lyophilized form. Examples of suitable pharmaceutical carriers are known in the art. Such carriers and/or additives can be formulated by conventional methods and can be administered to the subject at a suitable dose. Stabilizing agents such as lipids, nuclease inhibitors, polymers, and chelating agents can preserve the compositions from degradation within the body. In a composition intended to be administered by injection, one or more of a surfactant, preservative, wetting agent, dispersing agent, suspending agent, buffer, stabilizer and isotonic agent may be included.

The recombinant TCRs as described herein, or the viral vector particles comprising a nucleotide sequence encoding a recombinant TCR provided herein, can be packaged as kits. Kits can optionally include one or more components such as instructions for use, devices, and additional reagents, and components, such as tubes, containers and syringes for practice of the methods. Exemplary kits can include the nucleic acids encoding the recombinant TCRs, the recombinant TCR polypeptides, or viruses provided herein, and can optionally include instructions for use, a device for detecting a virus in a subject, a device for administering the compositions to a subject, and a device for administering the compositions to a subject.

Kits comprising polynucleotides encoding a gene of interest (e.g., a recombinant TCR) are also contemplated herein. Kits comprising a viral vector encoding a sequence of interest (e.g., a recombinant TCR) and optionally, a polynucleotide sequence encoding an immune checkpoint inhibitor are also contemplated herein.

Kits contemplated herein also include kits for carrying out the methods for detecting the presence of polynucleotides encoding any one or more of the TCRs disclosed herein. In particular, such diagnostic kits may include sets of appropriate amplification and detection primers and other associated reagents for performing deep sequencing to detect the polynucleotides encoding TCRs disclosed herein. In further embodiments, the kits herein may comprise reagents for detecting the TCRs disclosed herein, such as antibodies or other binding molecules. Diagnostic kits may also contain instructions for determining the presence of the polynucleotides encoding the TCRs disclosed herein or for determining the presence of the TCRs disclosed herein. A kit may also contain instructions. Instructions typically include a tangible expression describing the components included in the kit, and methods for administration, including methods for determining the proper state of the subject, the proper dosage amount, and the proper administration method. Instructions can also include guidance for monitoring the subject over the duration of the treatment time.

Kits provided herein also can include a device for administering a composition described herein to a subject. Any of a variety of devices known in the art for administering medications or vaccines can be included in the kits provided herein. Exemplary devices include, but are not limited to, a hypodermic needle, an intravenous needle, a catheter, a needle-less injection device, an inhaler, and a liquid dispenser, such as an eyedropper. Typically, the device for administering a virus of the kit will be compatible with the virus of the kit; for example, a needle-less injection device such as a high-pressure injection device can be included in kits with viruses not damaged by high-pressure injection, but is typically not included in kits with viruses damaged by high pressure injection.

Kits provided herein also can include a device for administering a compound, such as a T cell activator or stimulator, or a TLR agonist, such as a TLR4 agonist to a subject. Any of a variety of devices known in the art for administering medications to a subject can be included in the kits provided herein. Exemplary devices include a hypodermic needle, an intravenous needle, a catheter, a needle-less injection, but are not limited to, a hypodermic needle, an intravenous needle, a catheter, a needle-less injection device, an inhaler, and a liquid dispenser such as an eyedropper. Typically, the device for administering the compound of the kit will be compatible with the desired method of administration of the compound.

In particular embodiments, formulation of pharmaceutically-acceptable carrier solutions is well-known to those of skill in the art, as is the development of suitable dosing and treatment regimens for using the particular compositions described herein in a variety of treatment regimens, including e.g., enteral and parenteral, e.g., intravascular, intravenous, intrarterial, intraosseously, intraventricular, intracerebral, intracranial, intraspinal, intrathecal, and intramedullary administration and formulation. It would be understood by the skilled artisan that particular embodiments contemplated herein may comprise other formulations, such as those that are well known in the pharmaceutical art, and are described, for example, in Remington: The Science and Practice of Pharmacy, volume I and volume II. 22^{nd} Edition. Edited by Loyd V. Allen Jr. Philadelphia, PA: Pharmaceutical Press; 2012, which is incorporated by reference herein, in its entirety.

All publications, patent applications, and issued patents cited in this specification are herein incorporated by reference as if each individual publication, patent application, or issued patent were specifically and individually indicated to be incorporated by reference.

Although the foregoing embodiments have been described in some detail by way of illustration and example for purposes of clarity of understanding, it will be readily apparent to one of ordinary skill in the art in light of the teachings contemplated herein that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield essentially similar results.

### EXAMPLES

### Example 1

### MAGE-A4-TCR-Transgenic T Cells Bind MAGE-A4_{GVY}-MHC-Multimers

An in vitro priming approach was used to isolate MAGE-A4-reactive T-cell clones. The priming system used mature dendritic cells (mDCs) of HLA-A*02:01-positive as well as HLA-A*02:01-negative donors as antigen-presenting cells and autologous CD8+-enriched T cells as responding cells. In vitro transcribed RNA (ivtRNA) encoding the human MAGEA4 gene served as the source of specific antigen. After electroporation into the mDCs, the MAGE-A4-encoding ivtRNA was translated into protein, and subsequently processed and presented as peptides by HLA-A*02:01 encoded molecules on the mDCs. For HLA-A*02:01-negative donors, ivtRNA coding for HLA-A*02:01 was used in addition to MAGE-A4 ivtRNA to transgenically express the respective HLA allele in the antigen-presenting cells (allogeneic approach). In vitro co-cultures of T cells with the ivtRNA-transfected mDCs from the same donor lead to de novo induction of antigen-specific T cells that served as the source of corresponding TCRs. Antigen-specific T cells can be enriched by a variety of methods and are cloned by limiting dilution or FACS-based single cell sorting. Sequences of TCR alpha and TCR beta chains of MAGE-A4-reactive T-cell clones were identified by Next Generation Sequencing and after exchanging the constant TCR regions by their murine counterparts cloned into the retroviral vector pES.12-6. PBMCs of a healthy donor were isolated by ficoll gradient centrifugation. CD8+ T-cells were enriched by negative magnetic selection (Miltenyi) and stimulated in non-tissue culture 24-well plates, pre-coated with anti-CD3 (5 μg/ml) and anti-CD28 (1 μg/ml) mAb (BD Pharmingen, Heidelberg, Germany). Amphotropic retroviral particles were produced by transfection of HEK293T cells with the respective TCR encoding retroviral plasmid and two expression plasmids. On day two after stimulation, CD8+ T cells were transduced and on day twelve enriched for transduced CD8+ cells by FACS using the murine constant beta region as a marker for transduction and then expanded by rapid expansion protocol (Riddell SR, Science, 1992 Jul. 10;257(5067): 238-41).

In the experiments described in Examples 2-5, TCRs containing murinized Cα and Cβ regions were used (i.e., TCR-1 comprising the a chain of SEQ ID NO:57 and the β chain of SEQ ID NO: 58, TCR-2 comprising the α chain of SEQ ID NO:59 and the β chain of SEQ ID NO:60, TCR-3 comprising the α chain of SEQ ID NO:61 and the β chain of SEQ ID NO:62). The same types of experiments described in examples 2-5 could also be carried out using TCRs containing minimal murinized Cα and Cβ regions as described above.

### Results:

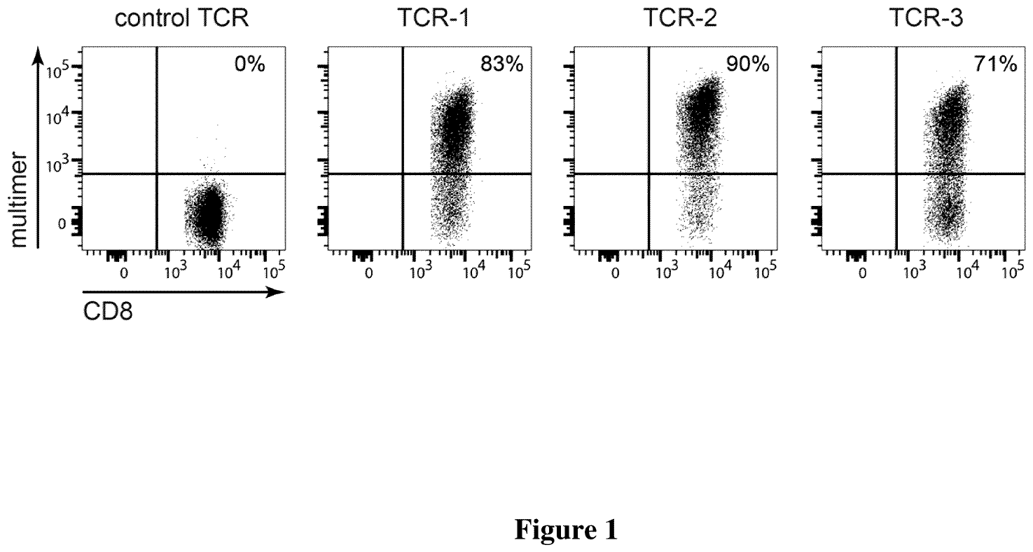

CD8+ T cells were transduced with three different TCRs isolated from MAGE-A4-reactive T-cell clones and one control TCR that did not recognize MAGE-A4. They were stained with a MAGE-A4_{GVY}-MHC-multimer (MAGE-A4²³⁰⁻²³⁹, GVYDGREHTV; immuneAware) and antibodies against CD8 and the murine constant beta region. All MAGE-A4-TCR-transgenic T cell populations bound the MAGE-A4_{GVY}-MHC-multimer very efficiently (>70%). No MAGE-A4_{GVY}-MHC-multimer-staining was observed with the control TCR. These results show that TCRs isolated from MAGE-A4-reactive T-cell clones can be transgenically expressed in T cells of a healthy donor (FIG. 1).

### Example 2

### MAGE-A4-TCR-Transgenic T Cells Recognize MAGE-A4_{GVY}-Peptide

MAGE-A4 specificity of TCR-transgenic T cells was confirmed according to the following protocol:

As target cells, T2 cells (HLA-A*02pos) were loaded with saturating amounts (10⁵ M) of MAGE-A4_{GVY}-peptide (SEQ ID NO: 1) or an irrelevant control peptide. In addition, K562 cells were transduced with HLA-A*02:01 and the MAGE-A4 gene (K562/A2/MAGE-A4). K562 cells transduced only with HLA-A*02:01 were used as a control (K562/A2). Each target cell line was co-cultured with TCR-transgenic T cells at a ratio of 2:1 using 20,000 T cells and 10,000 target cells. After 20-24 h, IFN-γ concentrations in co-culture supernatants were analyzed by standard sandwich ELISA (BD human IFN-α ELISA set).

### Results:

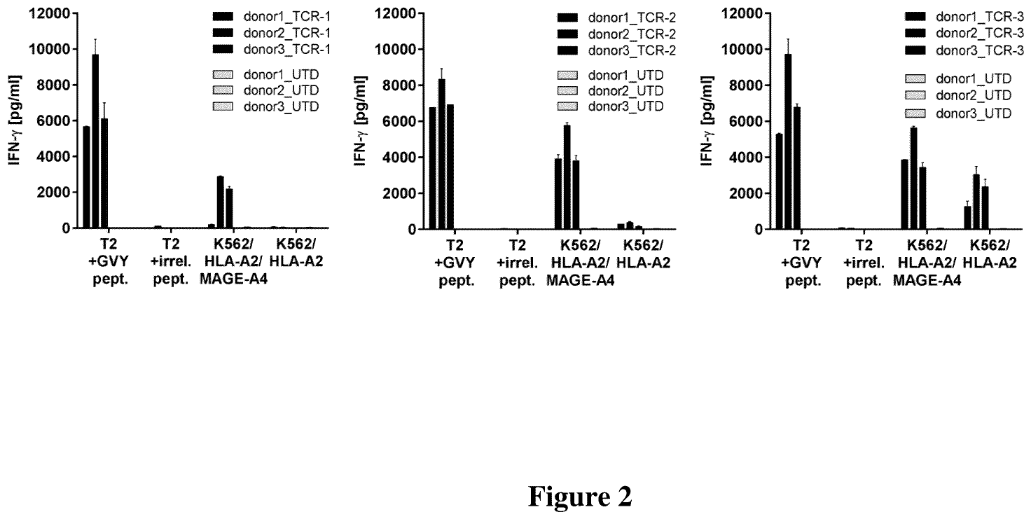

MAGE-A4-TCR-transgenic T cells recognized MAGE-A4_{GVY}-loaded T2 and MAGE-A4-transduced K562 cells but none of the control target cells. TCR-3-transduced T cells showed recognition of the K562/A2 control. These results show that TCRs isolated from MAGE-A4-reactive T-cell clones are functional when transferred to T cells of a healthy donor (FIG. 2).

### Example 3

### MAGE-A4-TCR-Transgenic T Cells Show High Functional Avidity

T2 cells loaded with MAGE-A4_{GVY}-peptide were used to analyze differences in the functional avidity of MAGE-A4-TCR-transgenic T cells:

T2 cells were externally loaded with graded concentrations (10⁻¹¹ M-10⁵ M) of the MAGE-A4_{GVY}-peptide and co-cultured with TCR-transgenic T cells at a ratio of 1:2 using 10,000 T2 cells and 20,000 T cells. After 20-24 h, IFN-γ concentrations in co-culture supernatants were analyzed by standard sandwich ELISA (BD human IFN-γ ELISA set).

### Results:

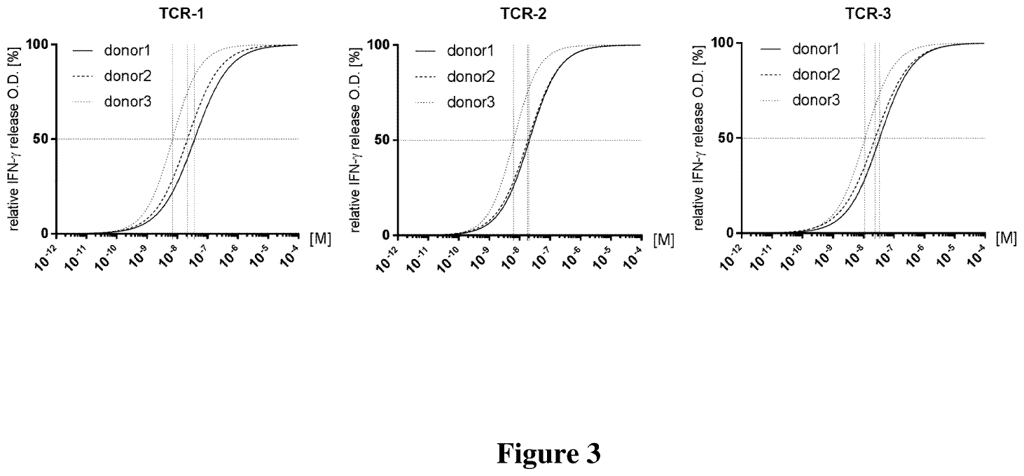

Integrated over multiple donors, the highest functional avidity against MAGE-A4_{GVY}-peptide loaded on HLA-A*02 was shown by TCR-2. TCR-1 and TCR-3 showed slightly reduced functional avidity compared to TCR-2 (FIG. 3).

### Example 4

### MAGE-A4-TCR-Transgenic T Cells Lyse MAGE-A4-Positive Tumor Cell Lines

MAGE-A4-positive HLA-A2-positive tumor cell lines (NCI-H1703, NCI-H1755), a MAGE-A4-negative HLA-A2-positive tumor cell line (Saos-2) and a MAGE-A4-negative HLA-A2-negative tumor cell line (A549) were used as target cells. For cytotoxicity assays, the co-cultures were set-up at an effector-to-target ratio of about 8-16:1 (depending on the target cell size), with 40,000 TCR-transgenic T cells and 5,000 (NCI-H1755, NCI-H1703, A549) and 2,500 (Saos-2) tumor cells respectively, that have been transduced with a fluorescent marker gene. Tumor cells loaded with saturated concentrations of MAGE-A4_{GVY}-peptide (10⁵ M) were used as internal positive control. The decrease of fluorescent target cells (cell count per well) was measured every three hours over a total time period of 172 hours using live-cell monitoring (IncuCyte® ZOOM, Essen Bioscience). To analyze cytokine release, co-culture supernatants were harvested after 24 h and respective IFN-γ concentrations were analyzed by standard sandwich ELISA (BD human IFN-γ ELISA set).

### Results:

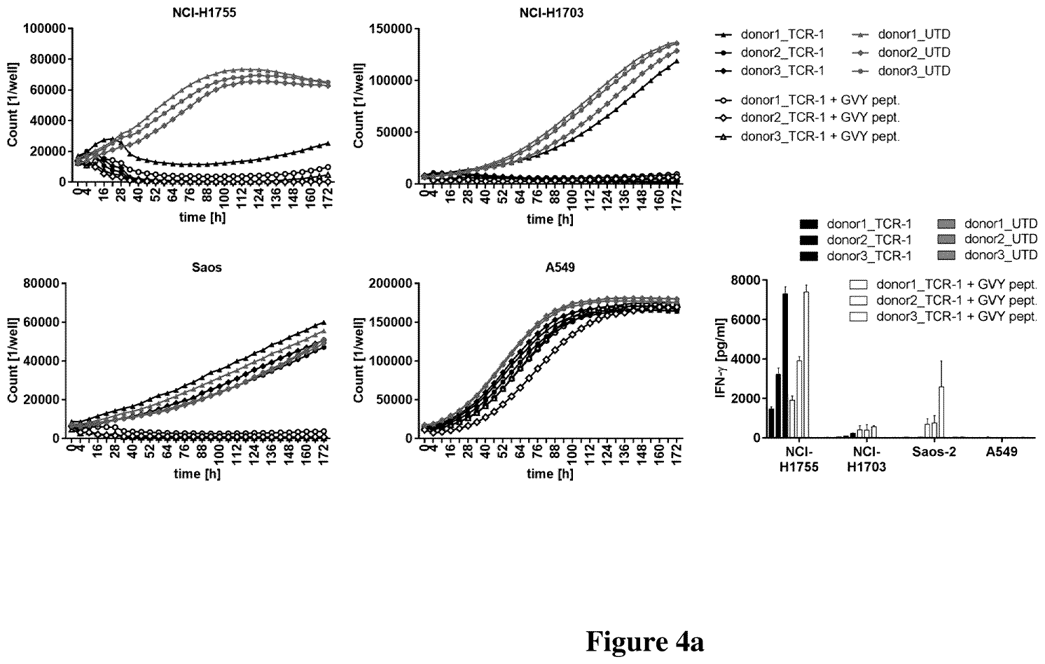

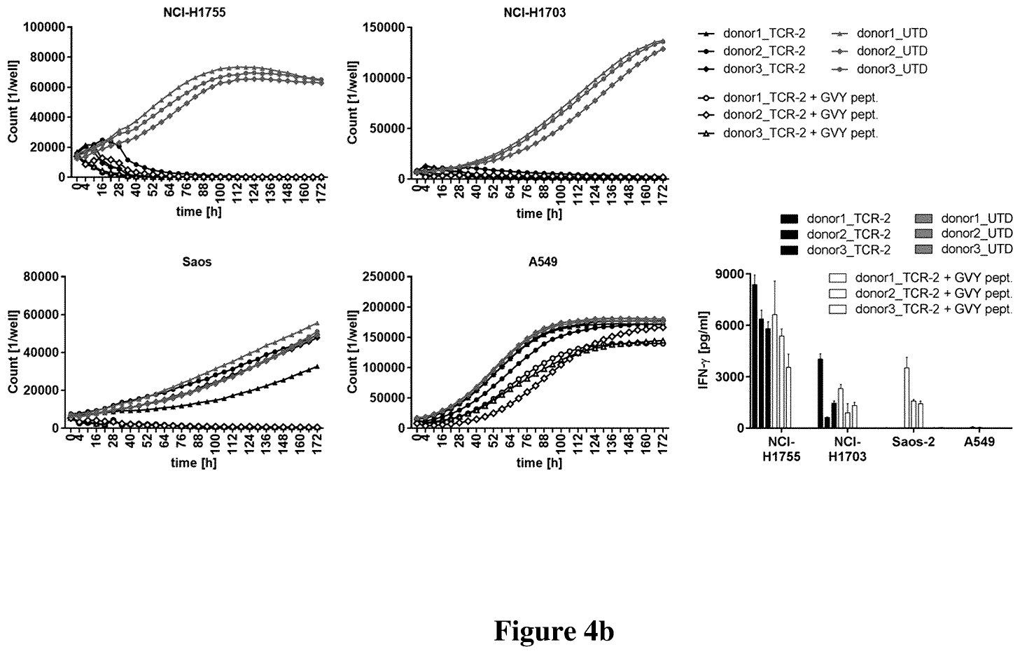

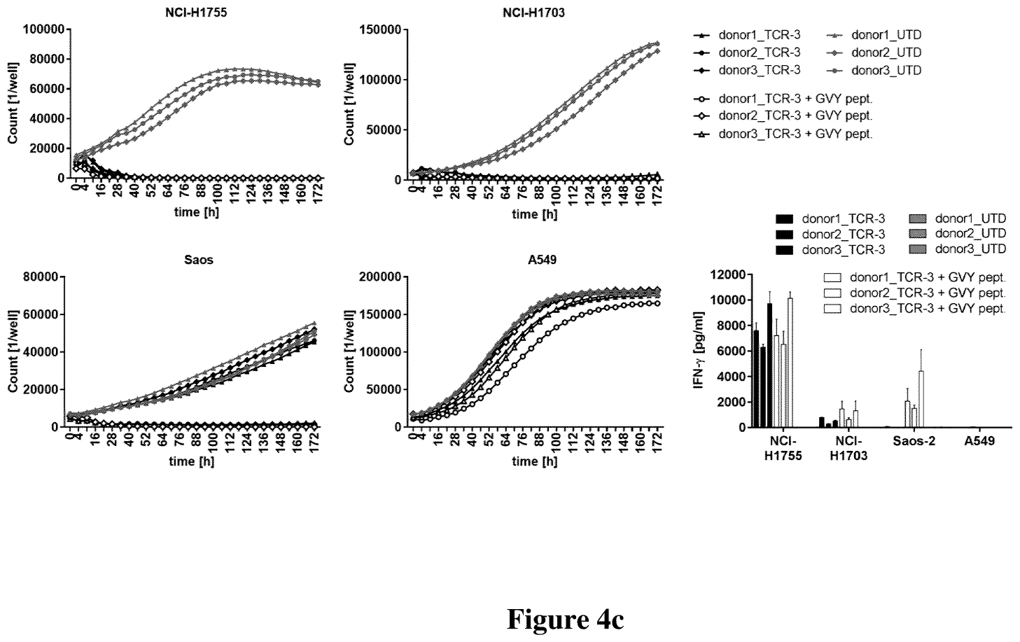

The two endogenously MAGE-A4-positive HLA-A2 positive tumor cell lines (NCI-H1703, NCI-H1755) were recognized and lysed by all MAGE-A4-TCR-transgenic T cells. The MAGE-A4-negative but HLA-A2 positive tumor cell line (Saos-2) was only recognized and lysed when the cells were externally loaded with saturated concentrations of MAGE-A4_{GVY}-peptide. The negative control cell line (A549) was neither recognized nor lysed by one of the MAGE-A4-TCRs. These results show that MAGE-A4-TCR-transgenic T cells can efficiently lyse endogenously MAGE-A4-positive tumor cells in a highly selective manner (FIGS. 4a, 4b and 4c).

### Example 5

### MAGE-A4-TCR-Transgenic T Cells Do Not Recognize Normal Human Cells

A panel of normal human cells was used to analyze potential on-target/off-tumor and off-target toxicities that could be caused by MAGE-A4-TCR-transgenic T cells.

Primary cells and induced pluripotent stem cell (iPS)-derived cells representing essential tissues or organs were tested for recognition by MAGE-A4-TCR-transduced T cells. HLA-A*02:01-negative NHBE cells were transfected with HLA-A2-ivtRNA via electroporation to transiently express HLA-A2. iCell Neurons were treated with IFN-γ for 72 h prior to start of the co-culture to induce cell surface HLA-A2 expression. HLA-A2 expression of all cell types was confirmed via flow cytometry. For toxicity assays, co-cultures were set-up with 20,000 TCR transgenic T cells and cell type specific amounts of target cells. As an internal positive control, all normal human cells were loaded with a final concentration of 10⁵ M of MAGE-A4_{GVY}-peptide. To analyze cytokine release, co-culture supernatants were harvested after 24 h and IFN-γ or IL-2 concentrations were analyzed by standard sandwich ELISA (BD human IFN-γor IL-2 ELISA set). IL-2 release was determined for co-culture with iCell Neurons, which have been pre-treated with IFN-γto induce HLA-A2 surface expression.

### Results:

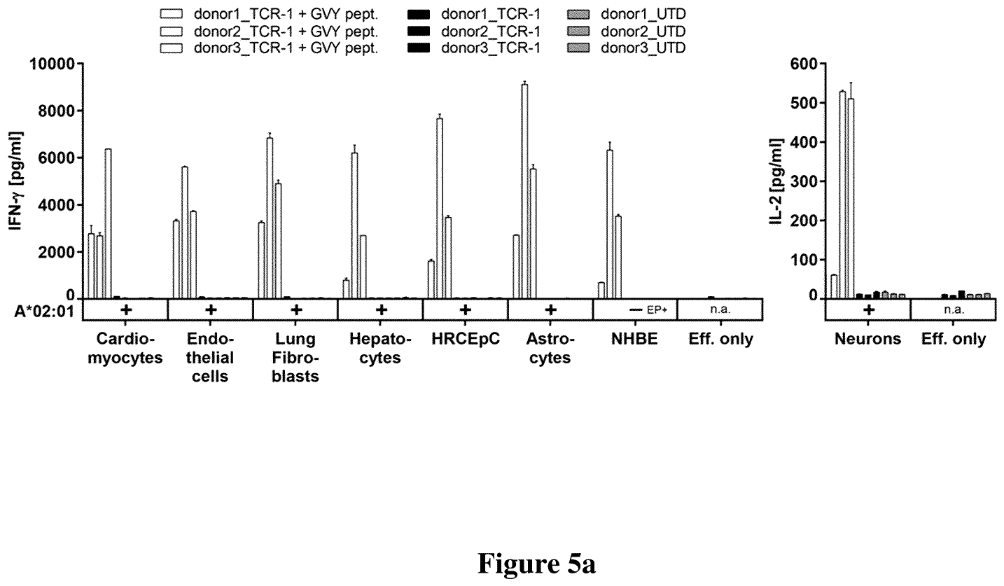

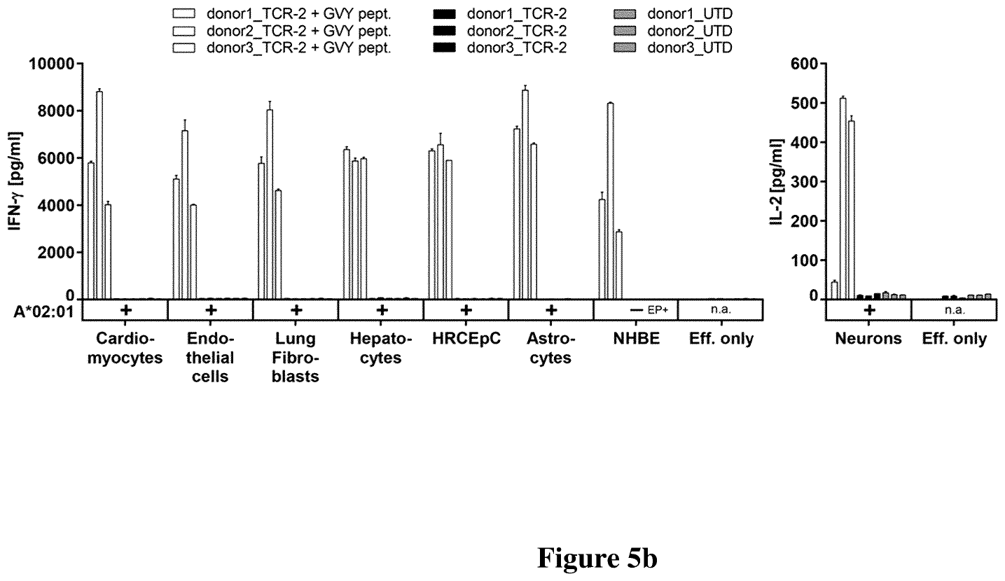

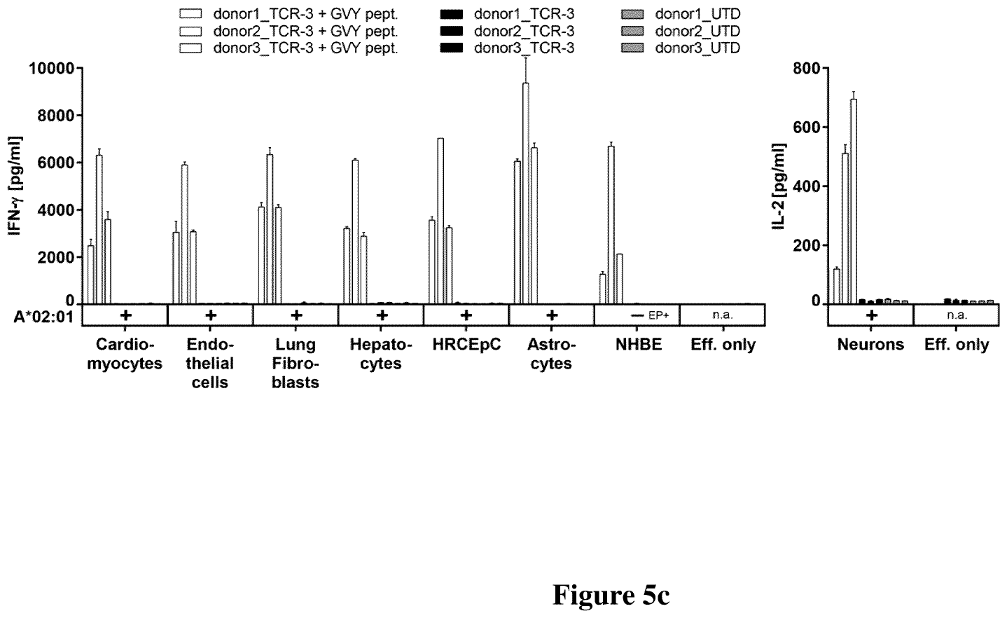

All primary cells and induced pluripotent stem cell (iPS)-derived cells were HLA-A2 positive at the beginning of co-cultivation with the MAGE-A4-TCR-transgenic T cells. Furthermore, the MAGE-A4-TCR-transgenic T cells were able to efficiently recognize all normal cells, when the individual target cells were loaded with the MAGE-A4_{GVY}-peptide. Unloaded normal cells were not recognized by any of the MAGE-A4-transgenic T cells. The MAGE-A4-transgenic T cells show no sign of on-target/off-tumor and off-target toxicities (FIGS. 5a, 5b and 5c).

### Example 6

### Lentiviral Vectors Encoding Fully Human MAGE-A4 TCRS

The TCR polynucleotide sequences identified in Example 1 were optimized for expression. Lentiviral vectors encoding polycistronic TCR constructs were used to express the TCRs. The polycistronic TCR constructs contain a TCR α or β chain, an optional furin cleavage site, a P2A ribosomal skipping sequence, and the corresponding TCR α or β chain. Lentiviral vectors were produced according to known methods. See e.g., Kutner et al., BMC Biotechnol. 2009;9:10. doi: 10.1186/1472-6750-9-10; Kutner et al. Nat. Protoc. 2009;4 (4): 495-505. doi: 10.1038/nprot.2009.22.

The polycistronic polynucleotide (SEQ ID NO: 93) encoding the MAGE-A4 TCR-4 polyprotein (SEQ ID NO: 94) contains a β chain encoded by SEQ ID NO: 70, a polynucleotide encoding a ribosomal skip sequence, and an a chain encoded by SEQ ID NO: 69.

The polycistronic polynucleotide (SEQ ID NO: 95) encoding the MAGE-A4 TCR-5 polyprotein (SEQ ID NO: 96) contains a β chain encoded by SEQ ID NO: 78, a polynucleotide encoding a furin cleavage site, a polynucleotide encoding a ribosomal skip sequence, and an α chain encoded by SEQ ID NO: 77.

The polycistronic polynucleotide (SEQ ID NO: 97) encoding the MAGE-A4 TCR-6 polyprotein (SEQ ID NO: 98) contains a β chain encoded by SEQ ID NO: 86, a polynucleotide encoding a furin cleavage site, a polynucleotide encoding a ribosomal skip sequence, and an α chain encoded by SEQ ID NO: 85.

### Example 7

### T Cells Expressing MAGE-A4 Fully Human TCRS Bind MAGE-A4_{GVY}-MHC-Multimers

CD3+ T cells were isolated from PBMCs of a healthy donor and transduced with lentiviral vectors encoding three different full human MAGE-A4 TCRs and a control TCR that did not recognize MAGE-A4. After expansion, the transduced T cells were stained with a MAGE-A4_{GVY}-MHC-multimer (MAGE-A4²³⁰⁻²³⁹, GVYDGREHTV; immuneAware) and antibodies against CD3. Populations were gated on live CD3+ cells and multimer staining. All MAGE-A4-TCR-transgenic T cell populations bound the MAGE-A4_{GVY}-MHC-multimer very efficiently (>70%). No MAGE-A4_{GVY}-MHC-multimer-staining was observed with the control TCR.

### Results:

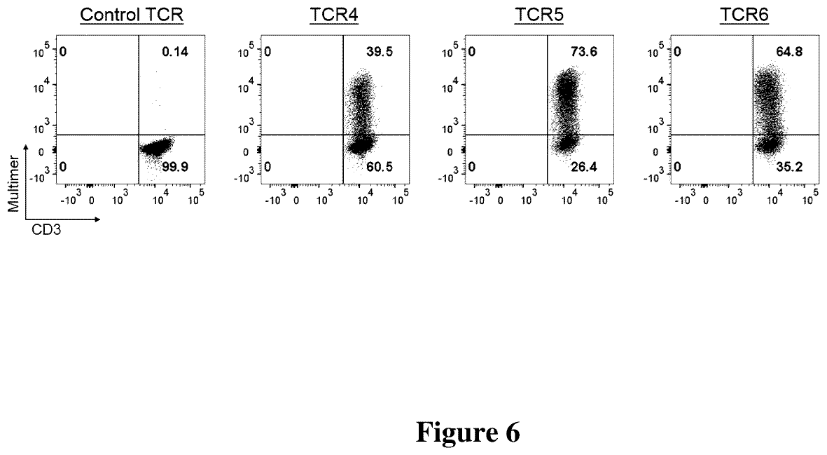

These results show that TCRs isolated from MAGE-A4-reactive T-cell clones can be transgenically expressed in T cells of a healthy donor (FIG. 6).

### Example 8

### T Cells Expressing MAGE-A4 Fully Human TCRS Recognize MAGE-A4_{GVY}-Peptide

MAGE-A4 specificity of TCR-transgenic T cells was confirmed using antigen dependent cytokine expression. T cells transduced with lentiviral vectors encoding the fully human MAGE-A4 TCRs described in Example 6 were co-cultured at an effector to target cell ratio of 2:1 with T2 cells (HLA-A*02pos) pulsed with 10 ng/ml of MAGE-A4_{GVY}-peptide or an irrelevant control peptide and with untransduced A549/HLA-A2 cells or A549/HLA-A2 transduced with the MAGE-A4 gene. After 20-24 hrs., IFN-γ concentrations in co-culture supernatants were analyzed using a Luminex assay.

### Results:

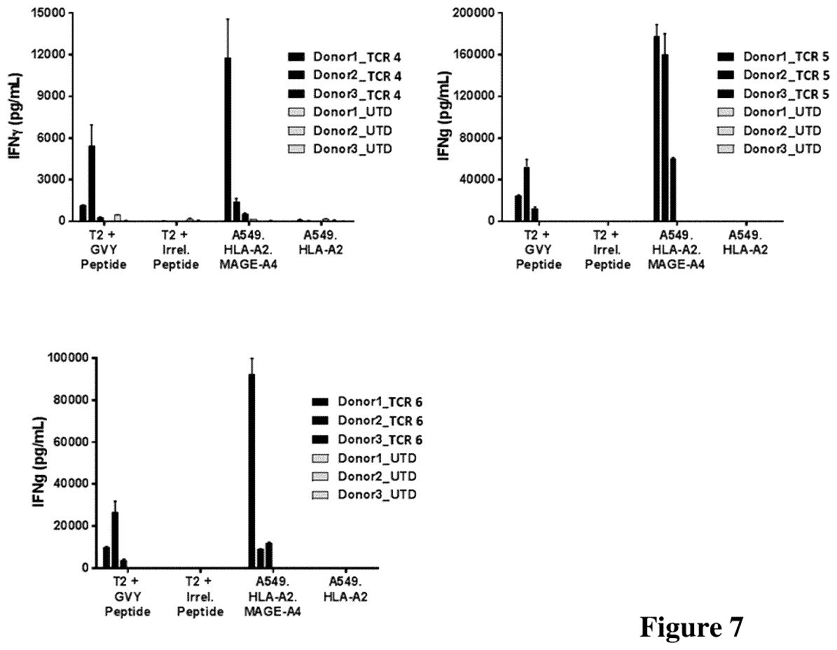

MAGE-A4-TCR-transgenic T cells recognized MAGE-A4_{GVY}-loaded T2 and MAGE-A4-transduced A549 cells but did not recognize the control target cells. These results show that healthy human donor T cells expressing fully human MAGE-A4 TCRs specifically react with target cells that display the MAGE-A4_{GVY} peptide (FIG. 7).

### Example 9

### T Cells Expressing MAGE-A4 Fully Human TCRS Show High Functional Avidity

Tumor cell lines that display the MAGE-A4_{GVY} peptide were used to analyze differences in the functional avidity of T cells expressing fully human MAGE-A4-TCRs. T cells transduced with lentiviral vectors encoding the fully human MAGE-A4 TCRs described in Example 6 were co-cultured at an effector to target cell ratio of 5:1 with MAGE-A4-positive HLA-A2-positive tumor cell lines (A375, NCI-H1703, NCI-H1755), a MAGE-A4-positive HLA-A2-negative tumor cell line (NCI-H520), and a MAGE-A4-negative HLA-A2-positive tumor cell line (A549). After 20-24 hrs., IFN-γ concentrations in co-culture supernatants were analyzed using a Luminex assay.

### Results:

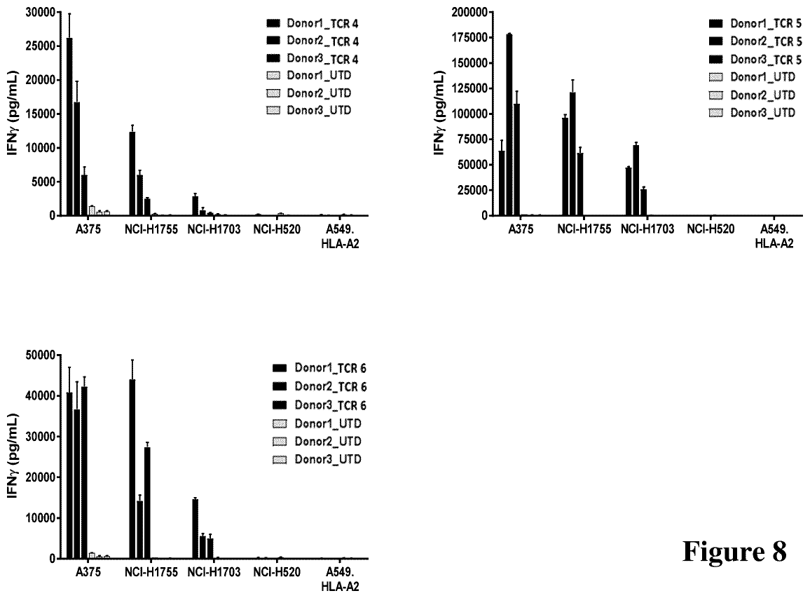

Integrated over multiple donors, MAGE-A4 TCR5 showed the highest functional avidity MAGE-A4-positive HLA-A2-positive tumor cell lines. MAGE-A4 TCR1 and MAGE-A4 TCR-6 showed reduced functional avidity compared to MAGE-A4 TCR-5 (FIG. 8).

### Example 10

### T Cells Expressing MAGE-A4 Fully Human TCRS Lyse MAGE-A4-Positive HLA-A2-Positive Tumor Cell Lines

T cells transduced with lentiviral vectors encoding the fully human MAGE-A4 TCRs described in Example 6 were co-cultured at an effector to target cell ratio of 5:1 with MAGE-A4-positive HLA-A2-positive tumor cell lines (A375, NCI-H1703, A549-HLA-A2-MAGE-A4) and a MAGE-A4-negative HLA-A2-positive tumor cell line (A549-HLA-A2). After 6 hrs. of co-culture, cytotoxicity against the tumor cell lines was measured by impedence assay.

### Results:

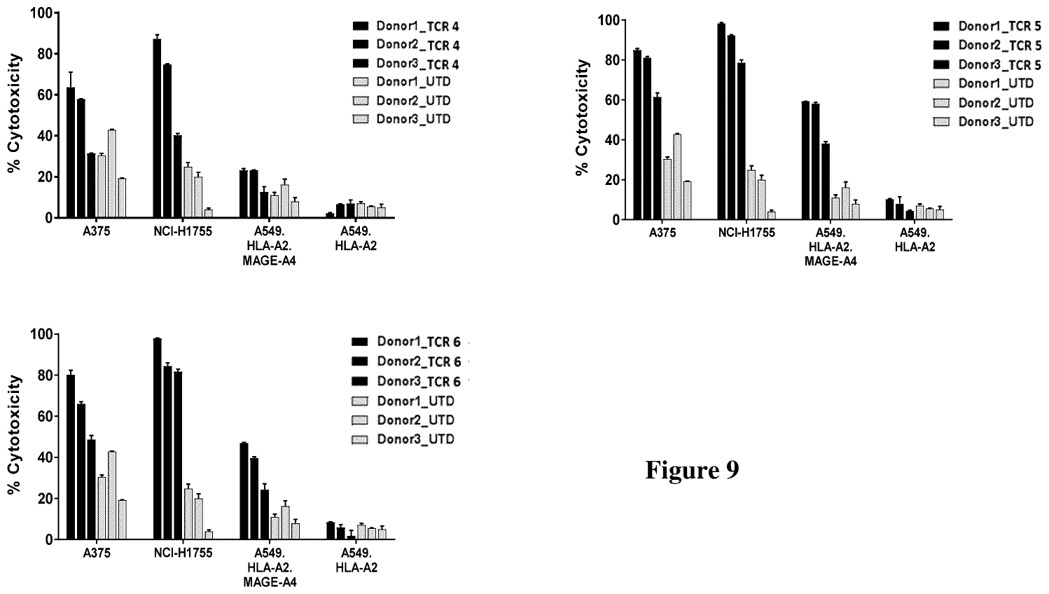

The MAGE-A4-positive HLA-A2 positive tumor cell lines (A375, NCI-H1703, A549-HLA-A2-MAGE-A4) were recognized and lysed by all MAGE-A4-TCR-transgenic T cells. Lysis of the MAGE-A4-negative HLA-A2-positive tumor cell line (A549-HLA-A2) MAGE-A4-TCR-transgenic T cells was not significantly different from the lysis observed using untransduced control T cells (FIG. 9).

### Example 11

### T Cells Expressing MAGE-A4 Fully Human TCRS Control MAGE-A4-Positive Tumors In Vivo

5×10⁶ MAGE-A4 positive A375 tumor cells were injected in each flank of 10 NSG mice. Ten days after tumor engraftment, 3.5×10⁷ MAGE-A4-TCR-transgenic T cells, 3.5×10⁷ control untransduced T cells or vehicle PBS were administered to the mice. After treatment, all mice had their tumor volumes measured twice a week by a caliper.

### Results:

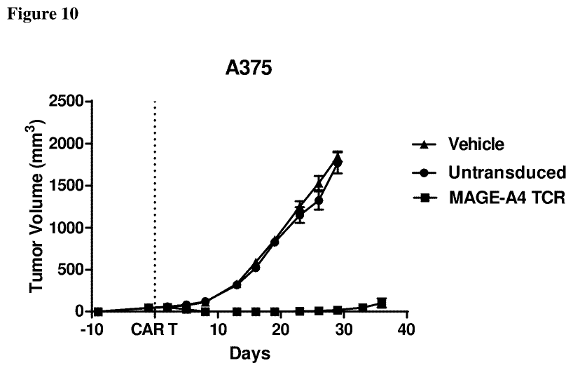

Untransduced T cell and vehicle-PBS treated mice failed to control tumor growth and were sacrificed once tumors reached maximum size permitted per protocol. MAGE-A4-TCR transgenic T cell treated mice controlled tumor growth for up to 35 days post T cell infusions (FIG. 10).

### Example 12

### Enhanced Human MAGE-A4 TCRS

The TCR polynucleotide sequences identified in Example 1 were modified to enhance expression and functional avidity. The TCR α and β chain constant regions were minimally murinized and hydrophobic amino acid substitutions were introduced into the transmembrane domain of TCR α chain constant region. Exemplary polynucleotide sequences for the enhanced MAGE-A4 TCRs are set forth in SEQ ID NOs: 97-99, 103-105, and 109-111. Exemplary polypeptide sequences for the enhanced MAGE-A4 TCRs are set forth in SEQ ID NOs: 100-102, 106-108, and 112-114.

Lentiviral vectors encoding polycistronic TCR constructs were used to express the enhanced MAGE-A4 TCRs (TCR-7, TCR-8, and TCR-9). The polycistronic TCR constructs contain a TCR α or β chain, an optional furin cleavage site, a P2A ribosomal skipping sequence, and the corresponding TCR α or β chain. Lentiviral vectors were produced according to known methods. See e.g., Kutner et al., BMC Biotechnol. 2009;9:10. doi: 10.1186/1472-6750-9-10; Kutner et al. Nat. Protoc. 2009;4(4): 495-505. doi: 10.1038/nprot.2009.22.

The polycistronic polynucleotide (SEQ ID NO: 101) encoding the MAGE-A4 TCR-7 polyprotein (SEQ ID NO: 104) contains a β chain encoded by SEQ ID NO: 100, a polynucleotide encoding a ribosomal skip sequence, and an α chain encoded by SEQ ID NO: 99.

The polycistronic polynucleotide (SEQ ID NO: 107) encoding the MAGE-A4 TCR-8 polyprotein (SEQ ID NO: 110) contains a β chain encoded by SEQ ID NO: 106, a polynucleotide encoding a furin cleavage site, a polynucleotide encoding a ribosomal skip sequence, and an a chain encoded by SEQ ID NO: 105.

The polycistronic polynucleotide (SEQ ID NO: 113) encoding the MAGE-A4 TCR-9 polyprotein (SEQ ID NO: 116) contains a β chain encoded by SEQ ID NO: 112, a polynucleotide encoding a furin cleavage site, a polynucleotide encoding a ribosomal skip sequence, and an α chain encoded by SEQ ID NO: 111.

### Example 13

### T Cells Expressing MAGE-A4 Fully Human TCRS or Enhanced MAGE-A4 TCRS can be Efficiently Expressed on Human T Cells.

Peripheral blood mononuclear cells (PBMCs) were isolated from three independent healthy donors, activated, and transduced with lentiviral vectors encoding a fully human MAGE-A4 TCR (TCR-5) or an enhanced MAGE-A4 TCR (TCR-8) or not transduced as a negative control. The cells were cultured for expansion in vitro and analyzed for vector integration by measuring vector copy number (VCN) and for expression by using flow cytometry against cells stained with a MAGE-A4_{GVY}-MHC-multimer (MAGE-A4²³⁰⁻²³⁹, GVYDGREHTV; immuneAware) and antibodies against CD3.

### Results:

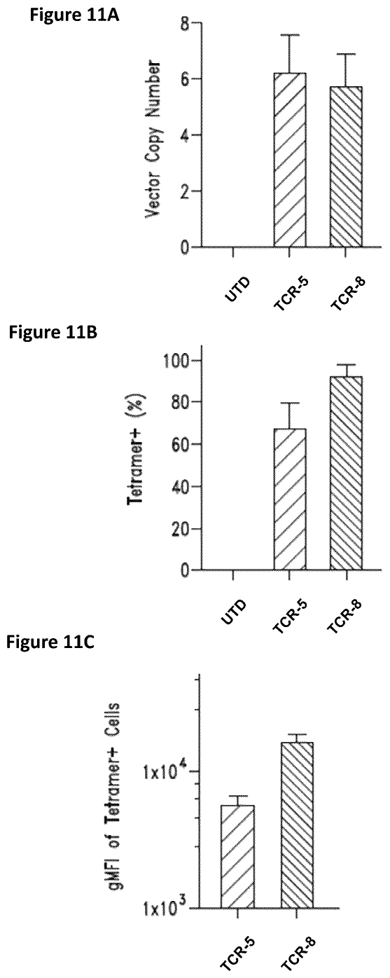

The VCNs for TCR-5 and TCR-8 were comparable, whereas TCR surface expression and density was higher in cells transduced with TCR-8 and with TCR-5. FIG. 11A-C.

### Example 14

### T Cells Expressing MAGE-A4 Fully Human TCRS or Enhanced MAGE-A4 TCRS Specifically Recognize and Kill MAGE-A4+ Cell Lines in Vitro

Peripheral blood mononuclear cells (PBMCs) were isolated from three independent healthy donors, activated, and transduced with lentiviral vectors encoding a fully human MAGE-A4 TCR (TCR-5) or an enhanced MAGE-A4 TCR (TCR-8) or untransduced (UTD) as a negative control. The TCR expressing T cells were evaluated for specific reactivity against MAGEA4 positive (+) and negative (-) tumor cell lines: A549.A2 (A2+, MAGE-A4(−)); NCI-H2023 (A2+, MAGE-A4(+)); A375 (A2+, MAGE-A4(+)); A549.A2.MAGEA4 (A2+, MAGE-A4(+)); and U2OS (A2+, MAGE-A4(low)).

### Results:

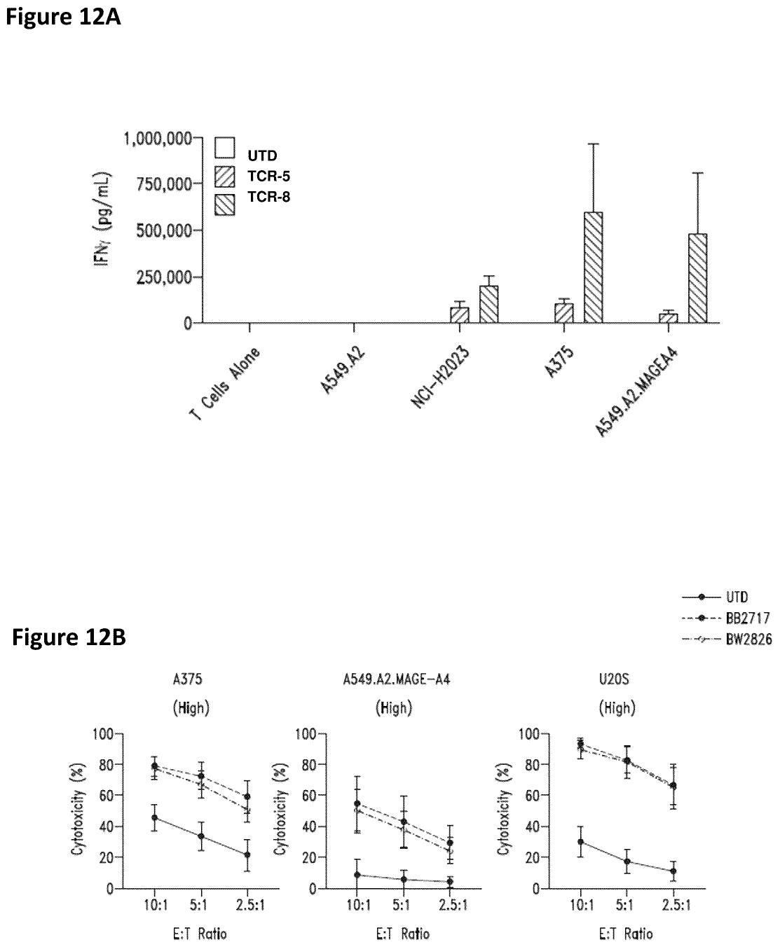

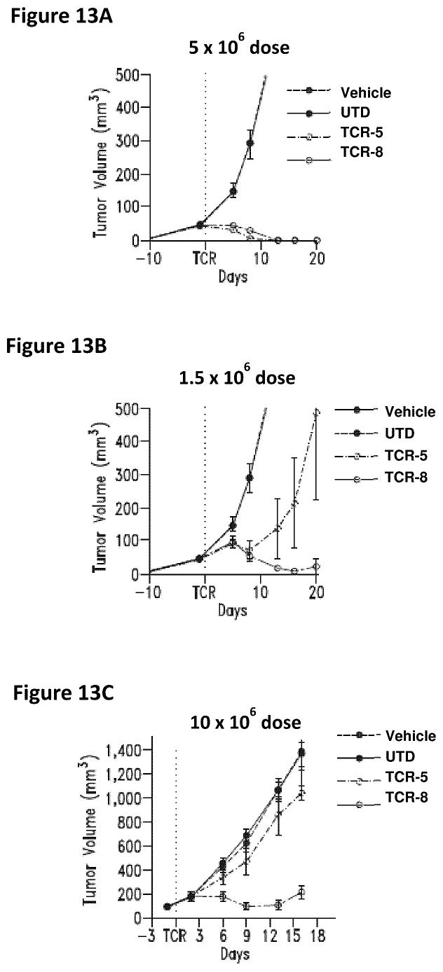

TCR-5 and TCR-8 T cells released IFNγ when co-cultured with HLA-A2+/MAGEA4(+) tumor cell lines but not when co-cultured with HLA-A2+/MAGEA4(−) cells or when cultured in the absence of target cell. UTD T cells did not release IFNγ in any culture conditions. FIG. 12A.

TCR-5 and TCR-8 T cells effectively killed HLA-A2+/MAGEA4(+) tumor cell lines at E:T ratios of 10:1, 5:1, and 2.5:1. UTD T cells did not kill HLA-A2+/MAGEA4(+) tumor cell lines at any E;T ratio. FIG. 12B.

### Example 15

T Cells Expressing MAGE-A4 Fully Human TCRS or Enhanced MAGE-A4 TCRS Mediate Regression of MAGE-A4 Expressing Tumors in Vivo

MAGE-A4 positive A375 tumor cells were injected in each flank of 5 NSG mice. Mice with 50 mm³ A375 tumors were administered PBS (Vehicle), untransduced T cells (UTD), 5×10⁶ TCR-5 or TCR-8 T cells (left flank), or 1.5×10⁶ TCR-5 or TCR-8 T cells (right flank). Mice with 100 mm³ A375 tumors were administered PBS (Vehicle), untransduced (UTD) T cells, or 10×10⁶ TCR-5 or

TCR-8 T cells. Tumor growth was measured twice a week and TCR T cells anti-tumor activity was evaluated in comparison to mice receiving UTD and Vehicle controls.

### Results:

TCR-5 and TCR-8 T cells mediated comparable tumor regression of 50 mm³ A375 tumors at a dose of 5×10⁶ TCR+ T cells. TCR-8 T cells mediated increased tumor regression compared to TCR-5 T cells of 50 mm³ A375 tumors at a dose of 1.5×10⁶ TCR+ T cells and of 100 mm³ A375 tumors at a dose of 10×10⁶ TCR+ T cells. Vehicle and UTD T cells did not mediate regression of A375 tumors in any condition.

The invention is further characterized by the following items:

Item 1: An isolated T cell receptor (TCR) specific for MAGE-A4.

Item 2: An isolated T cell receptor (TCR) specific for MAGE-A4, wherein the TCR comprises:
a) a variable TCR α region comprising a CDR1 having the amino acid sequence of SEQ ID NO: 2, a CDR2 having the amino acid sequence of SEQ ID NO: 3 and a CDR3 having the amino acid sequence of SEQ ID NO: 4,
a variable TCR β region comprising a CDR1 having the amino acid sequence of SEQ ID NO: 5, a CDR2 having the amino acid sequence of SEQ ID NO: 6 and a CDR3 having the amino acid sequence of SEQ ID NO: 7; or
b) a variable TCR α region comprising a CDR1 having the amino acid sequence of SEQ ID NO: 12, a CDR2 having the amino acid sequence of SEQ ID NO: 13 and a CDR3 having the amino acid sequence of SEQ ID NO: 14,
a variable TCR β region comprising a CDR1 having the amino acid sequence of SEQ ID NO: 15, a CDR2 having the amino acid sequence of SEQ ID NO: 16 and a CDR3 having the amino acid sequence of SEQ ID NO: 17; or
c) a variable TCR α region comprising a CDR1 having the amino acid sequence of SEQ ID NO: 22, a CDR2 having the amino acid sequence of SEQ ID NO: 23 and a CDR3 having the amino acid sequence of SEQ ID NO: 24,
a variable TCR β region comprising a CDR1 having the amino acid sequence of SEQ ID NO: 25, a CDR 2 having the amino acid sequence of SEQ ID NO: 26 and a CDR 3 having the amino acid sequence of SEQ ID NO: 27.

Item 3: The isolated TCR according to any of the preceding items, wherein the TCR specifically recognizes the amino acid sequence SEQ ID NO: 1 or a fragment thereof.

Item 4: The isolated TCR according to any of the preceding items, wherein the TCR specifically recognizes the HLA-A2 bound form of the amino acid sequence of SEQ ID NO: 1.

Item 5: The isolated TCR according to any of the preceding items, wherein the TCR specifically recognizes the amino acid sequence of SEQ ID NO: 1, which is presented by the HLA-A*02:01 encoded molecule.

Item 6: The isolated TCR according to any of the preceding items, wherein the TCR comprises a TCR α chain comprising a complementarity-determining region 3 (CDR3) having the sequence selected from the group consisting of SEQ ID NO: 4, SEQ ID NO: 14 and SEQ ID NO: 24.

Item 7: The isolated TCR according to any one of the preceding items, wherein the TCR comprises a TCR β chain comprising a CDR3 having the amino acid sequence selected from the group consisting of SEQ ID NO: 7, SEQ ID NO: 17 and SEQ ID NO: 27.

Item 8: The isolated TCR according to any one of the preceding items, wherein the TCR comprises
a) a variable TCR α region having an amino acid sequence which is at least 80% identical to SEQ ID NO: 8 and a variable TCR β region having an amino acid sequence which is at least 80% identical to SEQ ID NO: 9; or
b) a variable TCR α region having an amino acid sequence which is at least 80% identical to SEQ ID NO: 18 and a variable TCR β region having an amino acid sequence which is at least 80% identical to SEQ ID NO: 19; or
c) a variable TCR α region having an amino acid sequence which is at least 80% identical to SEQ ID NO: 28 and a variable TCR β region having an amino acid sequence which is at least 80% identical to SEQ ID NO: 29

Item 9: The isolated TCR according to any one of the preceding items, wherein the TCR comprises
a) a variable TCR α region having the amino acid sequence of SEQ ID NO: 8 and a variable TCR β region having the amino acid sequence of SEQ ID NO: 9; or
b) a variable TCR α region having the amino acid sequence of SEQ ID NO: 18 and a variable TCR β region having the amino acid sequence of SEQ ID NO: 19; or
c) a variable TCR α region having the amino acid sequence of SEQ ID NO: 28 and a variable TCR β region having the amino acid sequence of SEQ ID NO: 29.

Item 10: The isolated TCR according to any one of the preceding items, wherein the TCR comprises
a) a TCR α chain having an amino acid sequence which is at least 80% identical to SEQ ID NO: 10 and a TCR β chain having an amino acid sequence which is at least 80% identical to SEQ ID NO: 11; or
b) a TCR α chain having an amino acid sequence which is at least 80% identical to SEQ ID NO: 20 and a TCR β chain having an amino acid sequence which is at least 80% identical to SEQ ID NO: 21; or
c) a TCR α chain having an amino acid sequence which is at least 80% identical to SEQ ID NO: 30 and a TCR β chain having an amino acid sequence which is at least 80% identical to SEQ ID NO: 31

Item 11: The isolated TCR according to any one of the preceding items, wherein the TCR comprises
a) a TCR α chain having the amino acid sequence of SEQ ID NO: 10 and a TCR β chain having the amino acid sequence of SEQ ID NO: 11; or
b) a TCR α chain having the amino acid sequence of SEQ ID NO: 20 and a TCR β chain having the amino acid sequence of SEQ ID NO: 21; or
c) a TCR α chain having the amino acid sequence of SEQ ID NO: 30 and a TCR β chain having the amino acid sequence of SEQ ID NO: 31.

Item 12: The isolated TCR according to any one of the preceding items, wherein the TCR comprises a TCR α chain and a TCR β chain, wherein
a) the variable TCR α region has an amino acid sequence which is at least 80% identical to SEQ ID NO: 8 and comprises a CDR3 encoded by the amino acid sequence set out in SEQ ID NO: 4
the variable TCR β region has an amino acid sequence which is at least 80% identical to SEQ ID NO: 9 and comprises a CDR3 encoded by the amino acid sequence set out in SEQ ID NO: 7; or
b) the variable TCR α region has an amino acid sequence which is at least 80% identical to SEQ ID NO: 18 and comprises a CDR3 encoded by the amino acid sequence set out in SEQ ID NO: 14; or
the variable TCR β region has an amino acid sequence which is at least 80% identical to SEQ ID NO: 19 and comprises a CDR3 encoded by the amino acid sequence set out in SEQ ID NO: 17; or
c) the variable TCR α region has an amino acid sequence which is at least 80% identical to SEQ ID NO: 28 and comprises a CDR3 encoded by the amino acid sequence set out in SEQ ID NO: 24; or
the variable TCR β region has an amino acid sequence which is at least 80% identical to SEQ ID NO: 29 and comprises a CDR3 encoded by the amino acid sequence set out in SEQ ID NO: 27.

Item 13: The isolated TCR of any one of items 1 to 5, wherein the TCR comprises:
a) a TCR α chain having the amino acid sequence of SEQ ID NO: 10 and a TCR β chain having the amino acid sequence of SEQ ID NO: 11;
b) a TCR α chain having the amino acid sequence of SEQ ID NO: 20 and a TCR β chain having the amino acid sequence of SEQ ID NO: 21; or
c) a TCR α chain having the amino acid sequence of SEQ ID NO: 30 and a TCR β chain having the amino acid sequence of SEQ ID NO: 31.

Item 14: The isolated TCR of any one of items 1 to 5, wherein the TCR comprises:
a) a TCR α chain having the amino acid sequence of SEQ ID NO: 87 and a TCR β chain having the amino acid sequence of SEQ ID NO: 88;
b) a TCR α chain having the amino acid sequence of SEQ ID NO: 89 and a TCR β chain having the amino acid sequence of SEQ ID NO: 90; or
c) a TCR α chain having the amino acid sequence of SEQ ID NO: 91 and a TCR β chain having the amino acid sequence of SEQ ID NO: 92.

Item 15: The isolated TCR of any one of items 1 to 5, wherein the TCR comprises:
a) a TCR α chain having the amino acid sequence of SEQ ID NO: 102 and a TCR β chain having the amino acid sequence of SEQ ID NO: 103;
b) a TCR α chain having the amino acid sequence of SEQ ID NO: 108 and a TCR β chain having the amino acid sequence of SEQ ID NO: 109; or
c) a TCR α chain having the amino acid sequence of SEQ ID NO: 114 and a TCR β chain having the amino acid sequence of SEQ ID NO: 115.

Item 16: The isolated TCR according to any one of the preceding items, wherein the TCR is purified.

Item 17: The isolated TCR according to any one of the preceding items, wherein its amino acid sequence comprises one or more phenotypically silent substitutions.

Item 18: The isolated TCR according to any one of the preceding items, wherein its amino acid sequence is modified to comprise a detectable label, a therapeutic agent or pharmacokinetic modifying moiety.

Item 19: The isolated TCR according to item 18, wherein the therapeutic agent is selected from the group consisting of an immune effector molecule, a cytotoxic agent and a radionuclide.

Item 20: The isolated TCR according to item 19, wherein the immune effector molecule is a cytokine.

Item 21: The isolated TCR according to any one of the preceding items, wherein the TCR is soluble or membrane bound.

Item 22: The isolated TCR according to item 18, wherein the pharmacokinetic modifying moiety is at least one polyethylene glycol repeating unit, at least one glycol group, at least one sialyl group or a combination thereof.

Item 23: The isolated TCR according to any one of the preceding items, wherein the TCR is of the single chain type, wherein the TCR α chain and the TCR β chain are linked by a linker sequence.

Item 24: The isolated TCR according to any one of items 1 to 23, wherein the TCR α chain or the TCR β chain is modified to comprise an epitope tag.

Item 25: The isolated polypeptide comprising a functional portion of the TCR of any one of items 1 to 24, wherein the functional portion comprises at least one of the amino acid sequences of SEQ ID NOs: 4, 7, 14, 17, 24 and 27.

Item 26: The isolated polypeptide according to item 25, wherein the functional portion comprises the TCR α variable chain and/or the TCR β variable chain.

Item 27: A fusion protein comprising a TCR α chain and a TCR β chain, wherein the fusion protein comprises the amino acid sequence set forth in any one of SEQ ID NOs: 94, 96, 98, 104, 110, and 116.

Item 28: A multivalent TCR complex comprising a least two TCRs as embodied in any one of items 1 to 24.

Item 29: The multivalent TCR complex of item 28, wherein at least one of said TCRs is associated with a therapeutic agent.

Item 30: The isolated TCR according to any one of items 1 to 24, polypeptide according to item 25 or 26, fusion protein according to item 27, multivalent TCR complex according to item 28 or 29, wherein IFN-γ secretion is induced by binding to the amino acid sequence of SEQ ID NO: 1, which is presented by the HLA-A*02:01 encoded molecule.

Item 31: A nucleic acid encoding a TCR according to any one of items 1 to 24, encoding the polypeptide according to item 25 or 26, or encoding the fusion protein according to item 27.

Item 32: The nucleic acid according to item 31, wherein the nucleic acid sequence encoding the TCRα chain is set forth in any one of SEQ ID NOs: 69, 77, 85, 99, 105, and 111.

Item 33: The nucleic acid according to item 31 or item 32, wherein the nucleic acid sequence encoding the TCRβ chain is set forth in any one of SEQ ID NOs: 70, 78, 86, 100, 106, and 112.

Item 34: The nucleic acid according to item 31, wherein the TCR comprises an α chain encoded by SEQ ID NO: 69 and a β chain encoded by SEQ ID NO: 70; an α chain encoded by SEQ ID NO: 77 and a β chain encoded by SEQ ID NO: 78; an α chain encoded by SEQ ID NO: 85 and a β chain encoded by SEQ ID NO: 86; an α chain encoded by SEQ ID NO: 99 and a β chain encoded by SEQ ID NO: 100; an α chain encoded by SEQ ID NO: 105 and a β chain encoded by SEQ ID NO: 106; or an α chain encoded by SEQ ID NO: 111 and a β chain encoded by SEQ ID NO: 112.

Item 35: The nucleic acid according to item 31, wherein the fusion protein is encoded by the nucleic acid sequence set forth in any one of SEQ ID NOs: 93, 95, 97, 101, 107, and 113.

Item 36: A vector comprising the nucleic acid of any one of items 31 to 35.

Item 37: A vector comprising a nucleic acid encoding (a) the polypeptide sequences set forth in SEQ ID NO: 87 and SEQ ID NO: 88; (b) the polypeptide sequences set forth in SEQ ID NO: 89 and SEQ ID NO: 90; (c) the polypeptide sequences set forth in SEQ ID NO: 91 and SEQ ID NO: 92; (d) the polypeptide sequences set forth in SEQ ID NO: 102 and SEQ ID NO: 103; (e) the polypeptide sequences set forth in SEQ ID NO: 108 and SEQ ID NO: 109; or (f) the polypeptide sequences set forth in SEQ ID NO: 114 and SEQ ID NO: 115.

Item 38: The vector according to item 36 or item 37, wherein the vector is an expression vector.

Item 39: The vector according to any one of items 36 to 38, wherein the vector is a retroviral vector.

Item 40: The according to any one of items 36 to 39, wherein the vector is a lentiviral vector.

Item 41: A cell expressing the TCR according to any one of items 1 to 24.

Item 42: A cell comprising the vector according to any one of items 36 to 40.

Item 43: The cell according to item 41 or item 3942 wherein the cell is isolated or non-naturally occurring.

Item 44: A cell comprising the nucleic acid according to any one of items 31 to 35 or the vector according to any one of items 36 to 40.

Item 45: The cell according to items 41 to 44, wherein the cell comprises:
a) an expression vector which comprises at least one nucleic acid as embodied in any one of items 28 to 32.
b) a first expression vector which comprises a nucleic acid encoding the alpha chain of the TCR as embodied in any one of the items 1 to 21, and a second expression vector which comprises a nucleic acid encoding the beta chain of a TCR as embodied in any one of the items 1 to 21.

Item 46: The cell according to any one of items 41 to 45, wherein the cell is a peripheral blood lymphocyte (PBL) or a peripheral blood mononuclear cell (PBMC).

Item 49: The cell according to any one of items 41 to 48, wherein the cell is a T cell.

Item 50: The cell according to any one of items 41 to 48, wherein the cell is a T cell.

Item 51: An antibody or antigen binding fragment thereof specifically binding to a portion of the TCR according to any one of items 1 to 24 that mediates specificity for MAGE-A4.

Item 52: The antibody according to item 51, wherein the portion of the TCR that mediates the MAGE-A4 specificity comprises the
a) CDR3 of the alpha chain of SEQ ID NO: 4 and/or the CDR3 of the beta chain of SEQ ID NO: 7 or;
b) CDR3 of the alpha chain of SEQ ID NO: 14 and/or the CDR3 of the beta chain of SEQ ID NO: 17 or;
c) CDR3 of the alpha chain of SEQ ID NO: 24 and/or the CDR3 of the beta chain of SEQ ID NO: 27.

Item 53: A composition comprising the TCR according to any one of items 1 to 24, the polypeptide according to item 25 or 26, the fusion protein according to item 27, the multivalent TCR complex according to item 28 or 29, the nucleic acid according to any one of items 31 to 35, the vector according to any one of items 36 to 40, the cell according to any one of items 41 to 50, or the antibody according to item 51 or 52.

Item 54: A pharmaceutical composition comprising a pharmaceutically acceptable carrier and the TCR according to any one of items 1 to 24, the polypeptide according to item 25 or 26, the fusion protein according to item 27, the multivalent TCR complex according to item 28 or 29, the nucleic acid according to any one of items 31 to 35, the vector according to any one of items 36 to 40, the cell according to any one of items 41 to 50, or the antibody according to item 51 or 52.

Item 55: A pharmaceutical composition comprising at least one pharmaceutically acceptable carrier and the cell according to any one of items 41 to 50.

Item 56: The TCR according to any one of items 1 to 24, the polypeptide according to item 25 or 26, the fusion protein according to item 27, the multivalent TCR complex according to item 28 or 29, the nucleic acid according to any one of items 31 to 35, the vector according to any one of items 36 to 40, the cell according to any one of items 41 to 50, the antibody according to item 51 or 52, the composition of claim 53, or the pharmaceutical composition of claim 54 or claim 55 for use as a medicament.

Item 57: The TCR according to any one of items 1 to 24, the polypeptide according to item 25 or 26, the fusion protein according to item 27, the multivalent TCR complex according to item 28 or 29, the nucleic acid according to any one of items 31 to 35, the vector according to any one of items 36 to 40, the cell according to any one of items 41 to 50, the antibody according to item 51 or 52, the composition of claim 53, or the pharmaceutical composition of claim 54 or claim 55 for use in the treatment of cancer.

Item 58: The TCR, the polypeptide, the fusion protein, the multivalent TCR complex, the nucleic acid, the cell, the antibody, the composition, or the pharmaceutical composition according to item 57, wherein the cancer is a hematological cancer or a solid tumor.

Item 59: The TCR, the polypeptide, the fusion protein, the multivalent TCR complex, the nucleic acid, the cell, the antibody, the composition, or the pharmaceutical composition according to item 57 or 58, wherein the cancer is selected from the group consisting of sarcoma, prostate cancer, uterine cancer, thyroid cancer, testicular cancer, renal cancer, pancreatic cancer, ovarian cancer, esophageal cancer, non-small-cell lung cancer, non-Hodgkin's lymphoma, multiple myeloma, melanoma, hepatocellular carcinoma, head and neck cancer, gastric cancer, endometrial cancer, colorectal cancer, cholangiocarcinoma, breast cancer, bladder cancer, myeloid leukemia and acute lymphoblastic leukemia.

Item 60: The TCR, the polypeptide, the fusion protein, the multivalent TCR complex, the nucleic acid, the cell, the antibody, the composition, or the pharmaceutical composition according to item 50 or 51, wherein the cancer is preferably selected from the group consisting of NSCLC, SCLC, breast, ovarian or colorectal cancer, sarcoma or osteosarcoma.

### REFERENCES

Allen, L D (Editor): Remington: The Science and Practice of Pharmacy. Volume I and II. Twenty-second edition. Pharmaceutical Press 2012.
Bhan, S, Chuang, A, Negi, S S, Glazer, C A & Califano, J A: MAGEA4 induces growth in normal oral keratinocytes by inhibiting growth arrest and apoptosis. Oncol. Rep. 2012 28, 1498-1502.
Brichard V G, Louahed J, Clay T M: Cancer regression and neurological toxicity cases after anti-MAGE-A3 TCR gene therapy. J Immunother 2013 36:79-81.
Cameron B J, Gerry A B, Dukes J, Harper J V, Kannan V, Bianchi F C, et al.: Identification of a Titin-derived HLA-A1-presented peptide as a cross-reactive target for engineered MAGE A3-directed Tcells. Sci Transl Med 2013 5: 197ra103.
Cribbs A P, Kennedy A, Gregory B, Brennan F M: Simplified production and concentration of lentiviral vectors to achieve high transduction in primary human T cells. BMC Biotechnol. 2013; 13:98.
Daudi S, Eng K H, Mhawech-Fauceglia P, Morrison C, Miliotto A,Beck A, Matsuzaki J, Tsuji T, Groman A, Gnjatic S et al.: Expression and immune responses to MAGE antigens predict survival in epithelial ovarian cancer. PLOS One 2014, 9: e104099.
Duffour M T, Chaux P, Lurquin C, Cornelis G, Boon T, van der Bruggen P:
A MAGE-A4 peptide presented by HLA-A2 is recognized by cytolytic T lymphocytes. Eur J Immunol. 1999 October; 29(10): 3329-37.
Engels B, Engelhard V H, Sidney J, Sette A, Binder D C, Liu R B, Kranz D M, Meredith S C, Rowley D A, Schreiber H: Relapse or eradication of cancer is predicted by peptide-major histocompatibility complex affinity. Cancer Cell 2013; 23(4): 516-526.
Flynn J K and Gorry P R. Stem memory T cells (TSCM)-their role in cancer and HIV immunotherapies. Clin Transl Immunology 2014; 3(7): e20.
Gargett T, Brown MP: The inducible caspase-9 suicide gene system as a “safety switch” to limit on-target, off-tumor toxicities of chimeric antigen receptor T cells. Front Pharmacol. 2014 Oct. 28;5:235.
Gattinoni L, Lugli E, Ji Y, Pos Z, Paulos C M, Quigley M F, Almeida J R, Gostick E, Yu Z, Carpenito C, Wang E, Douek D C, Price D A, June C H, Marincola F M, Roederer M, Restifo NP: A human memory T cell subset with stem cell-like properties. Nat Med. 2011;17 (10): 1290-1297.
Giudicelli, V, et al.: IMGT/LIGM-DB, the IMGT® comprehensive database of immunoglobulin and T cell receptor nucleotide sequences, Nucl. Acid Research 2006, 34, D781-D784.
Gure A O, Chua R, Williamson B, Gonen M, Ferrera C A, Gnjatic S, Ritter G, Simpson A J, Chen Y T, Old L J et al.: Cancer-testis genes are coordinately expressed and are markers of poor outcome in non-small cell lung cancer. Clin Cancer Res 2005, 11:8055-8062.
Invitrogen Corporation: Vector NTI Advance™ 10 DNA and protein sequence analysis software. User Manual 2004, 389-662.
Kageyama S, Ikeda H, Miyahara Y, Imai N, Ishihara M, Saito K et al.: Adoptive transfer of MAGE-A4 T-cell receptor gene-transduced lymphocytes in patients with recurrent esophageal cancer. Clin Cancer Res 2015 21:2268-77.
Kieback E, Charo J, Sommermeyer D, Blankenstein T, Uckert W: A safeguard eliminates T cell receptor gene-modified autoreactive T cells after adoptive transfer. PNAS 2008 105 (2) 623-628.
Kim Y D, Park H R, Song M H, Shin D H, Lee C H, Lee M K, Lee S Y: Pattern of cancer/testis antigen expression in lung cancer patients. Int J Mol Med 2012, 29:656-662.
Lefranc and Lefranc: T cell Receptor Factsbook, Academic Press 2001, Elsevier Ltd ISBN 0-12-441352-8.
Li M, Yuan Y H, Han Y, Liu Y X, Yan L, Wang Y, Gu J: Expression profile of cancer-testis genes in 121 human colorectal cancer tissue and adjacent normal tissue. Clin Cancer Res 2005, 11:1809-1814.
Linette G P, Stadtmauer E A, Maus M V, Rapoport A P, Levine B L, Emery L et al.: Cardiovascular toxicity and titin cross-reactivity of affinity-enhanced T cells in myeloma and melanoma. Blood (2013) 122:863-71.
Lugli E, Gattinoni L, Roberto A, Mavilio D, Price D A, Restifo N P, Roederer M: Identification, isolation and in vitro expansion of human and nonhuman primate T stem cell memory cells. Nat Protoc. 2013;8(1): 33-42.
Morgan R A, Chinnasamy N, Abate-Daga D, Gros A, Robbins P F, Zheng Z et al.: Cancer regression and neurological toxicity following anti-MAGE-A3 TCR gene therapy. J Immunother (2013) 36:133-51
Otte M, Zafrakas M, Riethdorf L, Pichlmeier U, Loning T, Janicke F, Pantel K: MAGE-A gene expression pattern in primary breast cancer. Cancer Res 2001, 61:6682-6687.

Riddell S R, Sommermeyer D, Berger C, Liu L S, Balakrishnan A, Salter A, Hudecek M, Maloney D G, Turtle C J: Adoptive therapy with chimeric antigen receptor-modified T cells of defined subset composition. Cancer J. 2014;20(2): 141-144.
Sambrook: Molecular Cloning—4th Edition, Cold Spring Harbor Laboratory Press 2012, ISBN 978-1-936113-42-2.
Sommermeyer D, Uckert W: Minimal amino acid exchange in human TCR constant regions fosters improved function of TCR gene-modified T cells. J Immunol. 2010; 184(11): 6223-6231.
Tajima K, Obata Y, Tamaki H, Yoshida M, Chen Y T, Scanlan M J, Old L J, Kuwano H, Takahashi T, Takahashi T et al.: Expression of cancer/testis (CT) antigens in lung cancer. Lung Cancer 2003, 42:23-33.
Thompson J F, Higgins D G, Gibson T J: CLUSTAL W: improving the sensitivity of progressive multiple sequence alignment through sequence weighting, position-specific gap penalties and weight matrix choice. Nucleic Acids Research 1994, 22:4673-4680.
Yamada R, Takahashi A, Torigoe T, Morita R, Tamura Y, Tsukahara T, Kanaseki T, Kubo T, Watarai K, Kondo T et al.: Preferential expression of cancer/testis genes in cancer stem-like cells: proposal of a novel sub-category, cancer/testis/stem gene. Tissue Antigens 2013, 81:428-434.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. An isolated T cell receptor (TCR) specific for MAGE-A4, wherein the TCR comprises:
a) a variable TCR α region comprising a CDR1 having the amino acid sequence of SEQ ID NO: 2, a CDR2 having the amino acid sequence of SEQ ID NO: 3 and a CDR3 having the amino acid sequence of SEQ ID NO: 4,
a variable TCR β region comprising a CDR1 having the amino acid sequence of SEQ ID NO: 5, a CDR2 having the amino acid sequence of SEQ ID NO: 6 and a CDR3 having the amino acid sequence of SEQ ID NO: 7; or
b) a variable TCR α region comprising a CDR1 having the amino acid sequence of SEQ ID NO: 12, a CDR2 having the amino acid sequence of SEQ ID NO: 13 and a CDR3 having the amino acid sequence of SEQ ID NO: 14,
a variable TCR β region comprising a CDR1 having the amino acid sequence of SEQ ID NO: 15, a CDR2 having the amino acid sequence of SEQ ID NO: 16 and a CDR3 having the amino acid sequence of SEQ ID NO: 17; or
c) a variable TCR α region comprising a CDR1 having the amino acid sequence of SEQ ID NO: 22, a CDR2 having the amino acid sequence of SEQ ID NO: 23 and a CDR3 having the amino acid sequence of SEQ ID NO: 24,
a variable TCR β region comprising a CDR1 having the amino acid sequence of SEQ ID NO: 25, a CDR 2 having the amino acid sequence of SEQ ID NO: 26 and a CDR 3 having the amino acid sequence of SEQ ID NO: 27.

2. The isolated TCR according to claim 1, wherein the TCR specifically recognizes the amino acid sequence SEQ ID NO: 1 or a fragment thereof, preferably wherein the TCR specifically recognizes the HLA-A2 bound form of the amino acid sequence of SEQ ID NO: 1, more preferably wherein the TCR specifically recognizes the amino acid sequence of SEQ ID NO: 1, which is presented by the HLA-A*02:01 encoded molecule.

3. The isolated TCR of claim 1, wherein the TCR comprises:
a) a variable TCR α region having the amino acid sequence of SEQ ID NO: 8 and a variable TCR β region having the amino acid sequence of SEQ ID NO: 9; or
b) a variable TCR α region having the amino acid sequence of SEQ ID NO: 18 and a variable TCR β region having the amino acid sequence of SEQ ID NO: 19; or
c) a variable TCR α region having the amino acid sequence of SEQ ID NO: 28 and a variable TCR β region having the amino acid sequence of SEQ ID NO: 29.

4. The isolated TCR of claim 1, wherein the TCR comprises a functional portion comprising at least one of the amino acid sequences of SEQ ID NOs: 4, 7, 14, 17, 24 and 27.

5. The isolated TCR of any one of claim 1, wherein the TCR comprises:
a) a TCR α chain having the amino acid sequence of SEQ ID NO: 10 and a TCR β chain having the amino acid sequence of SEQ ID NO: 11;
b) a TCR α chain having the amino acid sequence of SEQ ID NO: 20 and a TCR β chain having the amino acid sequence of SEQ ID NO: 21; or
c) a TCR α chain having the amino acid sequence of SEQ ID NO: 30 and a TCR β chain having the amino acid sequence of SEQ ID NO: 31.

6. The isolated TCR of any one of claim 1, wherein the TCR comprises:
a) a TCR α chain having the amino acid sequence of SEQ ID NO: 87 and a TCR β chain having the amino acid sequence of SEQ ID NO: 88;
b) a TCR α chain having the amino acid sequence of SEQ ID NO: 89 and a TCR β chain having the amino acid sequence of SEQ ID NO: 90; or
c) a TCR α chain having the amino acid sequence of SEQ ID NO: 91 and a TCR β chain having the amino acid sequence of SEQ ID NO: 92.

7. The isolated TCR of claim 1, wherein the TCR comprises:
a) a TCR α chain having the amino acid sequence of SEQ ID NO: 102 and a TCR β chain having the amino acid sequence of SEQ ID NO: 103;
b) a TCR α chain having the amino acid sequence of SEQ ID NO: 108 and a TCR β chain having the amino acid sequence of SEQ ID NO: 109; or
c) a TCR α chain having the amino acid sequence of SEQ ID NO: 114 and a TCR β chain having the amino acid sequence of SEQ ID NO: 115.

8. The isolated TCR of claim 1, wherein IFN-γ secretion is induced by binding to the amino acid sequence of SEQ ID NO: 1, which is presented by the HLA-A*02:01 encoded molecule.

9. The isolated TCR of claim 8, wherein the IFN-γ secretion induced by binding of the TCR expressed on an effector cell to the amino acid sequence of SEQ ID NO: 1, which is presented by the HLA-A*02:01 encoded molecule, may be more than 100 times higher, preferably 500 times higher, more preferably 2000 times higher when binding to the amino acid sequence of SEQ ID NO: 1, which is presented by the HLA-A*02:01 encoded molecule, compared to binding to an irrelevant peptide, which is presented by the HLA-A*02:01 encoded molecule.

10. A multivalent TCR complex comprising at least two TCRs of claim 1.

11. A fusion protein comprising a TCR α chain and a TCR β chain, wherein the fusion protein comprises the amino acid sequence set forth in any one of SEQ ID NOs: 94, 96, 98, 104, 110, and 116.

12.-26. (canceled)

27. A method of treating cancer in a subject, comprising administering the TCR of claim 1, wherein the cancer is preferably a hematological cancer or a solid tumor, more preferably wherein the cancer is selected from the group consisting of sarcoma, prostate cancer, uterine cancer, thyroid cancer, testicular cancer, renal cancer, pancreatic cancer, ovarian cancer, esophageal cancer, non-small-cell lung cancer, non-Hodgkin's lymphoma, multiple myeloma, melanoma, hepatocellular carcinoma, head and neck cancer, gastric cancer, endometrial cancer, colorectal cancer, cholangiocarcinoma, breast cancer, bladder cancer, myeloid leukemia and acute lymphoblastic leukemia, most preferably wherein the cancer is selected from the group consisting of NSCLC, SCLC, breast, ovarian or colorectal cancer, sarcoma or osteosarcoma.
